(19) United States
(12) Patent Application Publication
(10) Pub. No.: US 2023/0032473 A1
(43) Pub. Date: Feb. 2, 2023

(54) NAD(H) NANOPARTICLES AND METHODS OF USE

(71) Applicant: Wisconsin Alumni Research Foundation, Madison, WI (US)

(72) Inventors: Shaoqin Gong, Middleton, WI (US); Mingzhou Ye, Madison, WI (US); Yi Zhao, Madison, WI (US)

(21) Appl. No.: 17/741,298

(22) Filed: May 10, 2022

Related U.S. Application Data
(60) Provisional application No. 63/225,372, filed on Jul. 23, 2021.

(51) Int. Cl. A61K 31/7084; A61K 9/127; A61K 47/02; A61K 47/22; A61K 31/496; A61K 31/192; A61K 31/65; A61K 31/7052; A61K 31/7036; A61K 31/513; A61K 31/522; A61K 31/4174; A61K 31/4196; A61K 31/506; A61P 31/04

(52) U.S. Cl. CPC A61K 31/7084; A61K 9/1271; A61K 47/02; A61K 47/22; A61K 31/496; A61K 31/192; A61K 31/65; A61K 31/7052; A61K 31/7036; A61K 31/513; A61K 31/522; A61K 31/4174; A61K 31/4196; A61K 31/506; A61P 31/04

(57) ABSTRACT

The present technology provides nanoparticles comprising an inorganic core and NAD⁺ or NADH, coated with a lipid bilayer, wherein the inorganic core is selected from calcium phosphate or a metal organic framework (MOF); the MOF comprises a transition metal ion coordinated to a coordinating ligand, wherein the transition metal ion is selected from the group consisting of zinc, iron, zirconium, copper, and cobalt ions, and the coordinating ligand is selected from an imidazolate ligand or a carboxylate ligand; and the nanoparticle has an average hydrodynamic diameter of from at least 50 nm to less than 1000 nm. Pharmaceutical compositions incorporating such nanoparticles and methods of treating sepsis and/or inflammation with such particles are also provided.

### CROSS-REFERENCE TO RELATED APPLICATIONS

This application claims the benefit of and priority to U.S. Patent Application No. 63/225,372, filed on Jul. 23, 2021, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 29, 2022, is named 032026-1488_SL.txt and is 18,275 bytes in size.

### FIELD

The present technology relates generally to the field of nanoparticles for delivery of NAD(H) (i.e., NAD⁺ and/or NADH) and, optionally, antimicrobials (e.g., antibiotic, antiviral or antifungal agents). The nanoparticles include an inorganic core (e.g., calcium phosphate or a metal organic framework) and are coated with a lipid bilayer with or without a PEG modified surface (see FIG. 1A). Methods of preparing and using the nanoparticles are also provided.

### BACKGROUND

Sepsis is a complex, life threatening disorder caused by a dysregulated host response to infection, resulting in about 20% of deaths annually worldwide, including nearly 270,000 deaths annually in the U.S. alone. Hyperinflammation and immunosuppression are the dominant features responsible for the high mortality of sepsis, which cannot be adequately addressed by current therapeutic strategies such as broad-spectrum antibiotic therapy or fluid resuscitation. Moreover, the dysregulated immune environment will cause endothelial apoptosis and dysfunction, thus prompting the progress of sepsis.

Nicotinamide adenine dinucleotide (NAD⁺) is known as an immune modulator; however, its relationship to inflammation is not yet fully understood. For instance, blocking NAD⁺biosynthesis and supplying NAD⁺ precursors were both reported to downmodulate pro-inflammatory cytokine production in vitro. There are also contradictory reports in the literature, suggesting that NAD⁺ or its precursor may or may not be beneficial in the treatment of infections. Therefore, the in vivo therapeutic benefit of NAD⁺ for sepsis is still under debate.

NAD⁺ is a negatively charged, hydrophilic small molecule that can hardly pass through a cell membrane. It must be degraded extracellularly into its precursors, nicotinamide (NAM) or nicotinamide riboside (NR), to enter the cells and subsequently enhance NAD⁺ biosynthesis¹⁶. This limitation in NAD⁺ intracellular transportation drastically decreases the bioactivity of NAD⁺ and would require extremely high doses for any proposed therapy. This may lead to inconsistent therapeutic outcomes and will greatly limit its potential for clinical use. Furthermore, although NAM can enhance intracellular NAD⁺ biosynthesis, it also showed opposite effects from NAD⁺ on a variety of important signals relevant to inflammation (e.g., sirtuins and poly (ADP-ribose) polymerase (PARP)). While attempts have been made to deliver NAD⁺ in vitro using nanovectors, none have been used for in vivo applications due to undesirable drug loading and release profiles.

### SUMMARY

The present technology provides nanoparticles (NPs) that include the oxidized and/or reduced forms of nicotinamide dinucleotide, i.e., NAD⁺ or NADH, respectively, or NAD(H) for short. These NPs show good biocompatibility and are capable of directly delivering NAD(H) (as opposed to NAD(H) precursors that must be metabolized to NAD(H); see FIG. 1A) into the cytosol of cells and are much stronger immune modulators compared with free NAD(H). They prevent cell pyroptosis and suppress inflammation by blocking both canonical and non-canonical NLRP3 inflammasome pathways. In addition, the NAD(H)-loaded NPs improve cellular energy supply and inhibit cell apoptosis and dysfunction in immune cells and endothelial cells, thus preventing immunosuppression and endothelial damage, two pivotal factors in the pathophysiology of sepsis.

In one aspect, the present technology provides a nanoparticle comprising an inorganic core and NAD⁺ or NADH, coated with a lipid bilayer, wherein the inorganic core may be calcium phosphate or a metal organic framework (MOF). In any embodiments, the inorganic core is coated with a cell membrane that comprises the lipid bilayer. The MOF comprises a transition metal ion coordinated to a coordinating ligand, wherein the transition metal ion is selected from the group consisting of zinc, iron, zirconium, copper, and cobalt ions, and the coordinating ligand is selected from an imidazolate ligand or a carboxylate ligand. All or a portion of the lipids in the lipid bilayer may be conjugated with poly(ethylene glycol) (PEG). The nanoparticle may have an average hydrodynamic diameter of from at least 50 nm to less than 1000 nm.

In another aspect, the present technology provides a pharmaceutical composition comprising any of the nanoparticles described herein.

In yet another aspect, the present technology provides a method of treating sepsis and/or inflammation comprising administering an effective amount of any nanoparticle described herein to a subject suffering from sepsis and/or inflammation. Where the sepsis and/or inflammation result from an infection (e.g., bacterial, viral or fungal infection), the methods further include administering an effective amount of an antimicrobial (e.g., antibiotic, antiviral, or antifungal agent) to the subject. The antimicrobial may be administered separately, simultaneously or sequentially with the nanoparticle.

In yet another aspect, the present technology provides a method of decreasing a level of TNF-α or IL-6 in a cell, comprising administering an effective amount of any of the nanoparticles described herein to the cell. The cell may be in vitro or in vivo.

### BRIEF DESCRIPTION OF THE DRAWINGS

FIGS. 1A-1I. NAD(H)-loaded NPs replenished cellular NAD(H) pool and prevented inflammation-induced energy depletion. FIG. 1A: Schematic illustration of an illustrative embodiment of NAD(H)-loaded NPs, NAD⁺ metabolism, and cellular uptake. FIG. 1B, FIG. 1C: Size and morphology of NAD+-LP-CaP (FIG. 1B) and NADH-LP-MOF (FIG. 1C) characterized by DLS and TEM. Scale bar: 200 nm. FIG. 1D, FIG. 1E: The NAD(H) release profiles from NAD+-LP-CaP (FIG. 1D) and NADH-LP-MOF (FIG. 1E) under different pH values. Data are presented as mean±s.d. (n=3). FIG. 1F, FIG. 1G: Intracellular NAD(H) levels (FIG. 1F) and NAD+/NAD(H) ratio (FIG. 1G) in BMDMs incubated with free NAD(H) (10 μM) or an equivalent dose of the NPs. Data are presented as mean±s.d. (n=5). Except as indicated, statistical significance was calculated via one-way ANOVA with Tukey's post hoc test for all data in the drawings. *P<0.05, ***P<0.001. FIG. 1H, FIG. 1I: Quantification of intracellular ATP level (n=5) (FIG. 1H) and cell viability (n=6) (FIG. 1I) in an LPS-mediated energy depletion model. LPS (100 ng/mL)-stimulated BMDMs were treated with free NAD(H) (10 μM) or an equivalent dose of the NPs. Data are presented as mean±s.d. **P<0.01, ***P<0.001

FIGS. 2A-2C. Intracellular NAD(H) levels in RAW 264.7 (FIG. 2A), HUVECs (FIG. 2B), or HEK 293 cells (FIG. 2C) incubated with free NAD(H) (10 μM) or NAD(H)-loaded NPs with an equivalent NAD(H) dose of 10 μM. Data are presented as mean±s.d. (n=5). *P<0.05,***P<0.001.

FIGS. 3A-3C. Intracellular NAD(H) levels in BMDMs treated with (FIG. 3A) LPS (100 ng/mL), (FIG. 3B) hydrogen peroxide (0.5 mM), or (FIG. 3C) NAMPT inhibitor FK866 (20 nM) and incubated in the presence of free NAD(H) (10 μM) or NAD(H)-loaded NPs with an equivalent NAD(H) dose of 10 μM. Data are presented as mean±s.d. (n=5). **P<0.01, ***P<0.001.

FIGS. 4A-4C. Quantification of intracellular ATP level in healthy (FIG. 4A), hydrogen peroxide (0.5 mM)-stimulated (FIG. 4B), or FK866 (20 nM)-stimulated BMDMs (FIG. 4C) incubated with free NAD(H) (10 μM) or NAD(H)-loaded NPs with an equivalent NAD(H) dose of 10 μNI. Data are presented as mean±s.d. (n=5). *P<0.05, **P<0.01, ***P<0.001.

FIGS. 5A-5B. Quantification of cell viability of BMDMs incubated with free NAD(H) (10 μM) or NAD(H)-loaded NPs with an equivalent NAD(H) dose of 10 μM in the presence of (FIG. 5A) hydrogen peroxide (0.5 mM) or (FIG. 5B) FK866 (20 nM). Data are presented as mean s.d. (n=6). ***P<0.001.

FIGS. 6A-6L. NAD(H)-loaded NPs prevented inflammation-induced cell death. FIG. 6A, FIG. 6B, Pro-inflammatory cytokine TNF-α (FIG. 6A) and IL-6 (FIG. 6B) levels in BMDMs culture supernatant quantified by ELISA. LPS (100 ng/mL)-stimulated BMDMs were treated with free NAD(H) (10 μM) or an equivalent dose of the NPs. Data are presented as mean±s.d. (n=3). Statistical analyses were done relative to the LPS treatment group. *P<0.05, **P<0.01, ***P<0.001. FIGS. 6C-6F, Analysis of caspase1 activation (demonstrated by FLICA staining, n=5) (FIGS. 6C, 6D) and IL-10 release (n=3) (FIGS. 6E, 6F) indicating the activation of canonical (FIGS. 6C, 6E) and non-canonical (FIGS. 6D, 6F) inflammasome pathways. BMDMs were primed with LPS (100 ng/mL, 3 h) as signal 1, and then either incubated with ATP (2.5 mM, 1 h) (for the canonical pathway) or transfected with Lipofectamine 2000 complexed LPS (lipoLPS, 100 ng LPS/well, 3 h) (for the non-canonical pathway) as signal 2. Free NAD⁺, empty NPs, or the NAD⁺-LP-CaP NPs were added together with signal 1 or signal 2 or both. Data are presented as mean±s.d. Statistical analyses were done relative to the positive controls (LPS/ATP or LPS/lipoLPS). *P<0.05, **P<0.01, ***P<0.001. FIG. 6G, FIG. 6H, NF-κB p65 nuclear translocation observed by CLSM. BMDMs were pretreated with free NAD(H) or the NPs for 5 h, stimulated with LPS (100 ng/mL) for 1 h, and then immunostained for p65. Representative images are presented. Scale bar: 20 μm. Data are presented as mean±s.d. (n=5). Statistical analyses were done relative to the LPS treatment group. *P<0.05, ***P<0.001. FIG. 6I, FIG. 6J, BMDM apoptosis triggered by LPS (100 ng/mL, 48 h) analyzed by Annexin V/PI double staining. FIG. 6K, HUVEC apoptosis triggered by TNF-α (80 ng/mL, 48 h). The cells were treated with free NAD(H) or the NPs at a corresponding dose of 10 μNI. FIG. 6L, Fluorescence images of HUVEC monolayer stained for tight junction protein VE-cadherin (green) after incubation with LPS together with free NAD(H) or the NPs for 24 h. Cell nuclei were stained by DAPI (blue). Representative images are presented. Scale bar: 50 μm.

FIGS. FIGS. 7A-7B. LDH release from BMDMs treated with free NAD⁺, empty LP-CaP (eCaP), or NADtLP-CaP (CaP) indicating cell pyroptosis triggered by canonical (FIG. 7A) and non-canonical (FIG. 7B) inflammasome pathways. BMDMs were primed with LPS (100 ng/mL, 3 h) as signal 1, and then either incubated with ATP (2.5 mM, 1 h) (for the canonical pathway) or transfected with Lipofectamine complexed LPS (lipoLPS, 100 ng LPS/well, 3 h) (for the non-canonical pathway) as signal 2. Free NAD⁺, empty LP-CaP, or the NADtLP-CaP were added together with signal 1 or signal 2 or both. Data are presented as mean±s.d. (n=5). ***P<0.001.

FIGS. 8A-8F. Analysis of LDH release (n=5) (FIG. 8A, FIG. 8B), caspase1 activation (demonstrated by FLICA staining, n=5) (FIG. 8C, FIG. 8D), and IL-10 release (n=3) (FIG. 8E, FIG. 8F) demonstrating the activation of canonical (FIG. 8A, FIG. 8C, FIG. 8E) and non-canonical (FIG. 8B, FIG. 8D, FIG. 8F) inflammasome pathways. BMDMs were primed with LPS (100 ng/mL, 3 h) as signal 1 and then incubated with ATP (2.5 mM, 1 h) or transfected with lipoLPS (100 ng LPS/well, 3 h) as signal 2. Free NADH, empty LP-MOF (eMOF), or NADH-LP-MOF (MOF) were added together with signal 1 or signal 2 or both. Data are presented as mean±s.d. Statistical analyses were done relative to the positive controls (LPS/ATP or LPS/lipoLPS). *P<0.05, **P<0.01, ***P<0.001.

FIGS. 9A-9H. Gene expression (including caspase1, caspase11, NLRP3, and pro-IL-1(3) in BMDMs primed with LPS and stimulated with ATP or lipoLPS. BMDMs were primed with LPS (100 ng/mL, 3 h) as signal 1 and then incubated with ATP (2.5 mM, 1 h) or transfected with lipoLPS (100 ng LPS/well, 3 h) as signal 2. NAD⁺-LP-CaP (CaP) or NADH-LP-MOF (MOF) were added together with signal 1 or signal 2 or both. Data are presented as mean±s.d. (n=5). **P<0.01, ***P<0.001.

FIGS. 10A-10B. Representative immunofluorescence images of ASC speck in BMDMs primed with LPS and stimulated with ATP (FIG. 10A) or lipoLPS (FIG. 10B). BMDMs were primed with LPS (100 ng/mL, 3 h) and then stimulated with either ATP (2.5 mM) for 1 h or lipoLPS (1 μg LPS/dish) for 3 h. Different treatments, including free NAD(H) (10 μM), empty NPs, or NAD(H)-loaded NPs with an equivalent NAD(H) dose of 10 μM, were given together with the stimuli. Representative images are presented. Scale bar: 20 μm.

FIGS. 11A-11B. Quantitative analysis of hydrogen peroxide (FIG. 11A) or FK866 (FIG. 11B) triggered apoptosis in BMDMs. BMDMs apoptosis triggered by hydrogen peroxide (0.5 mM, 12 h) or FK866 (20 nM, 48 h) were analyzed by Annexin V/PI double staining. The cells were treated with free NAD(H) (10 μM), empty NPs, or NAD(H)-loaded NPs (an equivalent NAD(H) dose of 10 μM).

FIG. 12. Mitochondrial membrane potential (ΔΨM) in BMDMs incubated with free NAD(H) (10 empty NPs, or NAD(H)-loaded NPs (an equivalent NAD(H) dose of 10 μM) in the presence of LPS (100 ng/mL, 24 h), hydrogen peroxide (0.5 mM, 6 h), or FK866 (20 nM, 24 h). Representative images are presented. Scale bar: 20 μm.

FIGS. 13A-13B. Quantitative analysis of LPS (FIG. 13A) or FK866 (FIG. 13B) triggered apoptosis in HUVECs. HUVECs apoptosis triggered by LPS (100 ng/mL, 48 h) or FK866 (20 nM, 48 h) were analyzed by Annexin V/PI double staining. The cells were treated with free NAD(H) (10 μM), empty NPs, or NAD(H)-loaded NPs (an equivalent NAD(H) dose of 10 μM).

FIG. 14. Fluorescence images of HUVECs monolayer stained for tight junction protein VE-cadherin (green) after incubation with TNF-α (100 ng/mL) together with free NAD(H) (10 empty NPs, or NAD(H)-loaded NPs (an equivalent NAD(H) dose of 10 μM) for 24 h. Cell nuclei were stained by DAPI (blue). Representative images are presented. Scale bar: 50 μm.

FIGS. 15A-15J. The therapeutic efficacy of the NPs in the LPS-induced mouse sepsis (endotoxemia) model. FIG. 15A, Experimental procedures for the LPS-induced sepsis (endotoxemia) model. FIGS. 15B-15D, Survival (FIG. 15B, FIG. 15C) and body weight (FIG. 15D) analysis of the mice receiving free NAD(H) (20 mg/kg) or an equivalent dose of NP treatment 1 h after LPS (15 mg/kg) administration. Data are presented as mean±s.d. (n=10). Statistical significance was calculated via Log-rank test. ***P<0.001. FIG. 15E, FIG. 15F, Pro-inflammatory cytokine TNF-α and IL-10 levels in the serum of septic mice receiving different treatments were measured 6 h after LPS challenge. Data are presented as mean±s.d. (n=6). ***P<0.001. FIG. 15G, Gene expression in the white blood cells (WBCs) of the mice receiving different treatments over negative controls. FIG. 15H, Ex vivo fluorescence images representing the biodistribution of Cy5.5-labeled LP-CaP NP in healthy and septic mice 4 h after NP administration. L, K, S, Lu, H, and M represent liver, kidneys, spleen, lungs, heart, and thigh muscle, respectively. FIG. 15I, Quantitative analysis of the mean fluorescence intensity of the organ or tissue shown in the ex vivo image. Data are presented as mean±s.d. (n=3). *P<0.05, ***P<0.001. FIG. 15I, The vascular hyperpermeability in LPS-induced septic mice receiving different treatments. Evans blue dye was injected 12 h after LPS challenge, and the amount of the dye retained in the lungs was extracted and quantified. Data are presented as mean±s.d. (n=6). ***P<0.001.

FIG. 16. Body weight variations of the mice in the LPS-induced sepsis model treated with various formulations including PBS, free NAD(H) (10 μM), empty NPs, or NAD(H)-loaded NPs (an equivalent NAD(H) dose of 20 mg/kg) 1 h after LPS (15 mg/kg) injection.

FIGS. 17A-17B. FIG. 17A, Ex vivo fluorescence images representing the biodistribution of Cy5.5-labeled LP-CaP in healthy and LPS-induced septic mice 24 h after NP administration. L, K, S, Lu, H, and M represent liver, kidneys, spleen, lungs, heart, and thigh muscle, respectively.

FIG. 17B, Quantitative analysis of the mean fluorescence intensity of the organ or tissue shown in the ex vivo image. Data are presented as mean±s.d. (n=3). ***P<0.001.

FIG. 18. Representative histological images for tissue sections with H&E staining in the LPS-induced sepsis model treated with various formulations including PBS, free NAD(H) (10 μM), empty NPs, or NAD(H)-loaded NPs (an equivalent NAD(H) dose of 20 mg/kg). The mice were sacrificed 24 h after LPS administration, and different organs were collected for histological analysis. Representative images are presented. Scale bar: 200 μm.

FIGS. 19A-19F. Immune cell population variation, apoptosis, and caspase1 activation in the blood, lungs, and spleen of LPS-induced septic (i.e., endotoxemia) mice. FIG. 19A, FIG. 19B, Flow cytometric quantification of monocyte (CD45⁺CD11b⁺Ly6C⁺Ly6G⁻), neutrophil (CD45⁺CD11b⁺Ly6C⁺Ly6G⁺), CD4⁺ T cell (CD45⁺CD11b^{low}CD3⁺CD4⁺), and CD8⁺ T cell (CD45⁺CD11b^{low}CD3⁺CD8⁺) populations in blood of septic mice 24 h after LPS administration. Free NAD⁺ (20 mg/kg) or an equivalent dose of LP-CaP and NAD⁺-LP-CaP were IV injected 1 h after LPS (7.5 mg/kg, IV) administration. Healthy mice without LPS injection were used as the negative control. Data are presented as mean±s.d. (n=5). **P<0.01, ***P<0.001. FIG. 19C, Flow cytometric quantification of monocyte and neutrophil populations in the lungs of the septic mice with free NAD⁺, LP-CaP, or NAD⁺-LP-CaP treatment. Data are presented as mean±s.d. (n=5). *P<0.05, **P<0.01, ***P<0.001. FIG. 19D, Representative plots of cell apoptosis for splenic lymphocytes. FIG. 19E, FIG. 19F, Quantification of cell apoptosis (FIG. 19E) and caspase1 activation (FIG. 19F) for a variety of immune cells (monocyte and neutrophil in blood, and CD4⁺ and CD 8⁺ T cell in spleen) assessed by Annexin V/7AAD staining and FLICA assay, respectively. Data are presented as mean±s.d. (n=5). *P<0.05, ***P<0.001.

FIGS. 20A-20D. Flow cytometric quantification of monocyte (FIG. 20A), neutrophil (FIG. 20B), CD4⁺ T cell (FIG. 20C), and CD8⁺ T cell (FIG. 20D) populations in the blood of LPS-induced septic mice with free NADH (20 mg/kg), LP-MOF, or NADH-LP-MOF (an equivalent NADH dose of 20 mg/kg) treatments. Data are presented as mean±s.d. (n=5). *P<0.05, **P<0.01, ***P<0.001.

FIGS. 21A-21B, Immature neutrophil (SSC^{low}) ratio in the blood of LPS-induced septic mice with free NAD⁺ (20 mg/kg), LP-CaP, or NAD⁺-LP-CaP (an equivalent NAD⁺ dose of 20 mg/kg) treatments. FIG. 21B, Immature neutrophil ratio in the blood of LPS-induced septic mice with free NADH (20 mg/kg), LP-MOF, or NADH-LP-MOF (an equivalent NADH dose of 20 mg/kg) treatments. Data are presented as mean±s.d. (n=5). ***P<0.001.

FIGS. 22A-22B. Flow cytometric quantification of CD4⁺ T cell (FIG. 22A) and CD8⁺ T cell (FIG. 22B) populations in the lungs of LPS-induced septic mice with free NAD⁺ (20 mg/kg), LP-CaP, or NAD⁺-LP-CaP (an equivalent NAD⁺ dose of 20 mg/kg) treatments. Data are presented as mean±s.d. (n=5). ***P<0.001.

FIGS. 23A-23D. Flow cytometric quantification of monocyte (FIG. 23A), neutrophil (FIG. 23B), CD4⁺ T cell (FIG. 23C), and CD8⁺ T cell (FIG. 23D) populations in the lungs of LPS-induced septic mice with free NADH (20 mg/kg), LP-MOF, or NADH-LP-MOF (an equivalent NADH dose of 20 mg/kg) treatments. Data are presented as mean±s.d. (n=5). **P<0.01, ***P<0.001.

FIGS. 24A-24B. Quantification of caspase1 activation in CD4⁺ T cell (FIG. 24A) and CD8⁺ T cell (FIG. 24B) in the blood of LPS-induced septic mice with free NAD⁺ (20 mg/kg), LP-CaP, or NAD⁺-LP-CaP (an equivalent NAD⁺ dose of 20 mg/kg) treatments. Data are presented as mean±s.d. (n=5). *P<0.05, ***P<0.001.

FIGS. 25A-25F. Quantification of caspase1 activation in monocyte (FIG. 25A), neutrophil (FIG. 25B), CD4⁺ T cell (FIG. 25C), and CD8⁺ T cell (FIG. 25D) in the blood, and CD4⁺ T cell (FIG. 25E) and CD8⁺ T cell (FIG. 25F) in spleen of LPS-induced septic mice with free NADH (20 mg/kg), LP-MOF, or NADH-LP-MOF (an equivalent NADH dose of 20 mg/kg) treatments. Data are presented as mean±s.d. (n=5). *P<0.05, **P<0.01, ***P<0.001.

FIGS. 26A-26F. Quantification of cell apoptosis for CD4⁺ T cell (FIG. 26A) and CD8⁺ T cell (FIG. 26B) in the blood, and monocyte (FIG. 26C), neutrophil (FIG. 26D), CD4⁺ T cell (FIG. 26E), and CD8⁺ T cell (FIG. 26F) in the lungs of LPS-induced septic mice with free NAD⁺(20 mg/kg), LP-CaP, or NAD⁺-LP-CaP (an equivalent NAD⁺ dose of 20 mg/kg) treatments.

FIGS. 27A-27C. Quantification of cell apoptosis for monocyte, neutrophil, CD4⁺ T cell, and CD8⁺ T cell in the blood (FIG. 27A), lungs (FIG. 27B), or spleen (FIG. 27C) of LPS-induced septic mice with free NADH (20 mg/kg), LP-MOF, or NADH-LP-MOF (an equivalent NADH dose of 20 mg/kg) treatments.

FIGS. 28A-28I. Therapeutic performance of the NAD⁺-loaded NPs in bacteria-induced sepsis models. FIG. 28A, Experimental procedures for the cecal ligation and puncture (CLP) and P. aeruginosa secondary infection model. Mice subjected to CLP received two IV injections of free NAD⁺ (20 mg/kg) or an equivalent dose of LP-CaP and NAD⁺-LP-CaP 6 h and 24 h after the surgery and challenged intratracheally with P. aeruginosa (1×10⁸CFU in 50 μL PBS) at day 3. A Sham group without CLP, and a CLP group without P. aeruginosa challenge were used as control groups. FIG. 28B, FIG. 28C, Survival (FIG. 28B) and body weight (FIG. 28C) analysis of the mice in the bacteria secondary infection model. Data are presented as mean±s.d. (n=14). Statistical significance was calculated via Log-rank test. **P<0.01. FIG. 28D, Experimental procedures for the MRSA and P. aeruginosa-induced polymicrobial blood infection model. Mixed MDR bacteria (mixed MRSA and P. aeruginosa with 5×10⁷ CFU for each pathogen) were IV administrated to induce blood infection and sepsis, and one injection of different treatments, including PBS, NAD⁺-LP-CaP, free rifampicin (acting as a model antibiotic), Rif-LP-CaP, or NAD⁺-Rif-LP-CaP at a dose corresponding to 10 mg rifampicin/kg and 20 mg NAD⁺/kg were IV injected 6 h after the infection. FIG. 28E, FIG. 28F, Survival (FIG. 28E) and body weight (FIG. 28F) analysis of the mice in the blood infection model. Data are presented as mean±s.d. (n=10). Statistical significance was calculated via Log-rank test. ***P<0.001. FIG. 28G, Bacterial loads in liver, spleen, lungs, kidneys, and blood of the septic mice 12 h after the treatments, determined by serial dilution and plate counting. Data are presented as mean±s.d. (n=6). Statistical analyses were done relative to the PBS treatment group. *P<0.05, **P<0.01, ***P<0.001. FIG. 28H, Blood biochemistry analysis demonstrating the liver (ALT, AST, ALP) and kidney (BUN) function of the mice with bacteria blood infection. Data are presented as mean±s.d. (n=3). Statistical analyses were done relative to the PBS treatment group. *P<0.05, **P<0.01, ***P<0.001. FIG. 28I, Representative histological images for tissue sections with haematoxylin and eosin (H&E) staining and TUNEL staining, from the healthy mice (control) and the infected mice with PBS or NAD⁺-Rif-LP-CaP treatments. Representative images are presented. Scale bar: 100 μm.

FIG. 29. Body weight variations of the mice in the cecal ligation and puncture (CLP) and P. aeruginosa secondary infection model. Two injections of free NAD⁺ (20 mg/kg per injection), LP-CaP, or NAD⁺-LP-CaP (equivalent NAD⁺ dose of 20 mg/kg per injection) were given through the tail vein 6 h and 24 h after CLP surgery. On day 3 post CLP, the surviving mice were anesthetized and infected with P. aeruginosa (1×10⁸ CFU in 50 μL PBS solution) through intratracheal injection.

FIG. 30. The Rif release profiles from NAD⁺-Rif-LP-CaP under different pH (5.5, 6.5, and 7.4) values (n=3).

FIG. 31. Body weight variations of the mice in the MRSA and P. aeruginosa-induced polymicrobial blood infection model.

FIGS. 32A-32B. Representative histological images for tissue sections with H&E staining (FIG. 32A) and TUNEL staining (FIG. 32B) in the MRSA and P. aeruginosa-induced polymicrobial blood infection model. Different treatments were given through IV injection 6 h after bacterial infection. The mice were sacrificed 24 h after infection and the organs were collected for staining. In the PBS treated group, multiple organ injury was observed, including congestion and dramatical inflammatory cell infiltration in the heart, spotty hepatocellular necrosis, nuclear debris in the spleen, alveolar wall thickening in the lungs, severe congestion in the outer stripe of the outer medulla (OSOM) of the kidney, and tubular necrosis and cell sloughing in the inner stripe of the outer medulla (ISOM) of the kidney. Representative images of three independent experiments. Scale bar for H&E staining: 200 μm. Scale bar for TUNEL staining: 100 μm.

FIG. 33. Blood biochemistry analysis of creatine kinase (CRE) in the MRSA and P. aeruginosa-induced polymicrobial blood infection model with PBS, NAD⁺-LP-CaP, free Rif, Rif-LP-CaP, and NAD⁺-Rif-LP-CaP (20 mg/kg NAD⁺ and 10 mg/kg Rif) treatment. Data are presented as mean±s.d. (n=5). *P<0.05, **P<0.01.

FIG. 34. Representative histological images for tissue sections from healthy mice subjected to three administrations of NAD⁺-LP-CaP, NAD⁺-Rif-LP-CaP, or NADH-LP-MOF every other day (containing 20 mg/kg NAD⁺ and 10 mg/kg Rif for each injection) via intravenous injections. Representative images are presented. Scale bar: 200 μm.

FIG. 35. In vitro cytotoxicity of NAD(H)-loaded NPs evaluated by MTT assay. Cell viability was tested in a 96-well plate seeded with 1×10⁴ BMDMs per well. BMDMs were incubated with different concentrations of NAD(H)-loaded NPs for 24 h. Data are presented as mean±s.d. (n=5). Statistical significance was calculated via one-way ANOVA with Tukey's post hoc test, and presented relative to the untreated group (labeled as zero NAD(H) concentration in the figure). *P<0.05, **P<0.01, ***P<0.001.

FIGS. 36A-36C. Macrophage polarization indicated by the relative mRNA expression of iNOS (FIG. 36A), Arg1 (FIG. 36B), and their ratio (iNOS/Arg1) (FIG. 36C). BMDMs were treated with LPS (100 ng/mL) in the presence of free NAD(H) (10 μM), empty NPs, or NAD(H)-loaded NPs with an equivalent NAD(H) dose of 10 μM. Data are presented as mean±s.d. (n=5). Statistical significance was calculated via one-way ANOVA with Tukey's post hoc test. ***P<0.001.

FIG. 37. Proinflammatory cytokine TNF-α levels in the neutrophil culture supernatant quantified by ELISA. LPS (100 ng/mL)-stimulated neutrophils were treated with free NAD(H) (10 μM), empty NPs, or NAD(H)-loaded NPs with an equivalent NAD(H) dose of 10 μM for 3 h. Data are presented as mean±s.d. (n=5). Statistical significance was calculated via one-way ANOVA with Tukey's post hoc test. ***P<0.001.

FIG. 38. Fluorescence images of neutrophils stained for NETs (Sytox green, green) after incubation with LPS (100 ng/mL) together with free NAD(H) (10 μM), empty NPs, or NAD(H)-loaded NPs (at an equivalent NAD(H) dose of 10 μM) for 3 h. Cell nuclei were stained by Hoechst 33342 (blue). Representative images are presented. Scale bar: 20 μm.

FIGS. 39A-39B. Cytosolic calcium levels in BMDMs with different treatments. BMDMs with (FIG. 39B) or without (FIG. 39A) LPS (100 ng/mL) stimulation were incubated with free NAD(H) (10 empty NPs, or NAD(H)-loaded NPs with an equivalent NAD(H) dose of 10 μM for 8 h. The cells were then stained with Fluo-3/AM (2 μM) for 0.5 h and monitored by flow cytometry. Data are presented as mean±s.d. (n=5). Statistical significance was calculated via one-way ANOVA with Tukey's post hoc test. **P<0.01, ***P<0.001. For the experiment without LPS stimulation, statistical analyses were done relative to the control group.

FIGS. 40A-40D. (FIG. 40A, FIG. 40b): Ex vivo fluorescence images representing the biodistribution of Cy5.5-labeled LP-MOF in healthy and septic mice 4 h (FIG. 40A, FIG. 40C) and 24 h (FIG. 40B, FIG. 40D) after NP administration. L, K, S, Lu, H, and M represent liver, kidneys, spleen, lungs, heart, and thigh muscle, respectively. (FIG. 40C, FIG. 40D): Quantitative analysis of the mean fluorescence intensity of the organ or tissue shown in the ex vivo images. Data are presented as mean±s.d. (n=3). Statistical significance was calculated via one-way ANOVA with Tukey's post hoc test. *P<0.05, ** P<0.01, ***P<0.001.

FIG. 41. ATP levels in different organs of the mice treated with LPS (15 mg/kg). Free NAD⁺ (20 mg/kg), or an equivalent dose of LP-CaP and NAD⁺-LP-CaP was given 1 h after LPS injection. PBS was injected as a control group. The mice were sacrificed 24 h after LPS administration, and different organs or tissues were collected, weighed, homogenized, and lysed for ATP quantification. Data are presented as mean±s.d. (n=5). Statistical significance was calculated via one-way ANOVA with Tukey's post hoc test. *P<0.05, ** P<0.01.

FIGS. 42A-42B. Stability study of the NAD(H)-loaded NPs. (FIG. 42A) The NPs were dispersed in an aqueous solution at 4° C., and their sizes were monitored by DLS at different time points. (FIG. 42B) The NPs lyophilized from an aqueous solution containing 10% sucrose as the cryoprotectant were stored at −20° C., and their sizes were tested once a week. Data are presented as mean±s.d. (n=3). Statistical significance was calculated via one-way ANOVA with Tukey's post hoc test.

### DETAILED DESCRIPTION

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “carboxyl” or “carboxylate” as used herein refers to a —COOH group or the salt thereof.

The term “imidazolyl” or “imidazolate” as used herein refers to a heterocyclic organic compound containing two nitrogen atoms separated by a carbon atom in a five-membered ring, (i.e., 1,3-diazole) or the salt thereof. Representative substituted imidazolyl groups may be substituted one or more times with substituents such as those listed above.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “hydroxyl” as used herein can refer to —OH or its ionized form, A “hydroxyalkyl” group is a hydroxyl-substituted alkyl group, such as HO—CH₂—.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York). Amino protecting groups include, but are not limited to, mesitylenesulfonyl (Mts), benzyloxycarbonyl (Cbz or Z), t-butyloxycarbonyl (Boc), t-butyldimethylsilyl (TBS or TBDMS), 9-fluorenylmethyloxycarbonyl (Fmoc), allyloxycarbonyl (Alloc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α,α-dimethyldimethoxybenzyloxycarbonyl (DDZ), 5-bromo-7-nitroindolinyl, and the like. Amino protecting groups susceptible to acid-mediated removal include but are not limited to Boc and TBDMS. Amino protecting groups resistant to acid-mediated removal and susceptible to hydrogen-mediated removal include but are not limited to Alloc, Cbz, nitro, and 2-chlorobenzyl oxy carbonyl.

As used herein, the terms “effective amount” or “therapeutically effective amount,” or “pharmaceutically effective amount” refer to a quantity sufficient to achieve a desired therapeutic and/or prophylactic effect, e.g., an amount which results in the full or partial amelioration of disease or disorders or symptoms associated with sepsis in a subject (e.g., a mammal) in need thereof. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. A person of ordinary skill in the art will be able to determine appropriate dosages depending on these and other factors. The compositions can also be administered in combination with one or more additional compounds. Multiple doses may be administered. Additionally or alternatively, multiple therapeutic compositions or compounds may administered. In the methods described herein, the compounds may be administered to a subject having one or more signs or symptoms of a disease or disorder described herein.

As used herein, the term “secondary infection” means any infection which may occur after a primary infection and/or inflammation and/or after a wound, such as postoperatively after a surgical wound. It may be for example an infection occurring 1 to 90 days after the beginning of a primary infection, for example 5 to 12 day after the beginning of a primary infection. It may be also for example an infection occurring 1 to 90 days after the end of a primary infection for example 5 to 12 day after the end of the primary infection and/or the absence of any pathological sign and/or symptom.

As used herein, the term “subject” refers to any animal that can experience sepsis, such as a mammal or a bird. In any embodiments, the mammal may be selected from primates, dogs, cats, rodents, horses, cattle, or pigs. In any embodiments, the subject (i.e., primate subject) is a human.

“Treating,” “treat,” “treated,” or “treatment” as used herein covers the treatment of a disease or disorder described herein (e.g., AD), in a subject, such as a human, and includes: (i) inhibiting or preventing a disease or disorder, i.e., arresting its development; (ii) relieving a disease or disorder, i.e., causing regression of the disorder; (iii) slowing progression of the disorder; and/or (iv) inhibiting, preventing, relieving, ameliorating, or slowing progression of one or more symptoms of the disease or disorder. Symptoms may be assessed by methods known in the art or described herein, for example, biopsy, histology, and blood tests to determine relevant enzyme levels, metabolites or circulating antigen or antibody (or other biomarkers), quality of life questionnaires, patient-reported symptom scores, and imaging tests.

“Ameliorate,” “ameliorating,” and the like, as used herein, refer to inhibiting, relieving, eliminating, or slowing progression of one or more symptoms.

“Metal organic framework” or “MOF” as used herein refers to the three-dimensional, porous, crystalline structure formed by metal ions and small organic ligands that coordinate to the metal ions. Thus, a “metal organic framework component” refers collectively to the individual component parts of the MOF, i.e., a metal ion and a coordinating ligand. For example, a zinc ion and 2-methylimidazole would be, respectively, the metal ion and coordinating ligand of the metal organic framework component for the MOF, zeolitic imidazolate framework-8, i.e., ZIF-8.

“Molecular weight” as used herein with respect to polymers refers to number-average molecular weights (Mₙ) and can be determined by techniques well known in the art including gel permeation chromatography (GPC). GPC analysis can be performed, for example, on a D6000M column calibrated with poly(methyl methacrylate) (PMMA) using triple detectors including a refractive index (RI) detector, a viscometer detector, and a light scattering detector, and N,N′-dimethylformamide (DMF) as the eluent. “Molecular weight” in reference to small molecules and not polymers is actual molecular weight, not number-average molecular weight.

The terms “preventing” and “prophylaxis” as used herein refer to administering a pharmaceutical compound or medicament or a composition including the pharmaceutical compound or medicament to a subject before a disease, disorder, or condition fully manifests itself, to forestall the appearance and/or reduce the severity of one or more symptoms of the disease, disorder or condition. The person of ordinary skill in the art recognizes that the term “prevent” is not an absolute term. In the medical art it is understood to refer to the prophylactic administration of a drug to diminish the likelihood or seriousness of a disease, disorder or condition, or a symptom thereof, and this is the sense that such terms are used in this disclosure.

The phrase “targeting ligand” refers to a ligand that binds to “a targeted receptor” that distinguishes the cell being targeted from other cells. The ligands may be capable of binding due to expression or preferential expression of a receptor for the ligand, accessible for ligand binding, on the target cells. While sepsis is a system wide condition that does not require a targeting ligand, other types of inflammation can benefit from nanoparticles of the present technology to which targeting ligands are attached. Examples of such ligands include various oligo and polysaccharides, e.g., mannan, heparain, levan, chondroitin sulfate, glycogen, alginate, and galactan (targeting infected tissues); galactose (targeting liver); those targeting macrophages such as mannose (targeting mannose receptor, CD206); ferritin (targeting ferritin receptor); and hyaluronic acid (targeting CD44 over-expressed macrophages); those targeting immune system cells such as MPLA (targeting TLR4), and imiquimod (targeting TLR7); those targeting endothelial cells such as anti-EGFR nanobody (targeting EGFR receptor); αvβ3 integrin ligand LXW7 (targeting αvβ3 integrin); αvβ3 integrin ligand GRGD peptide (SEQ ID NO: 1) (targeting αvβ3 integrin); thiophosphate modified aptamer or E-selectin binding peptide (DITWDQLWDLMK (SEQ ID NO: 2) or KYDGDITWDQLWDLMK (SEQ ID NO: 3)) (targeting E-Selectin).

The phrase “a targeted receptor” refers to a receptor expressed by a cell that is capable of binding a cell targeting ligand. The receptor may be expressed on the surface of the cell. The receptor may be a transmembrane receptor. Examples of such targeted receptors include galactose receptors expressed by hepatocytes, receptors for C-type lectins (e.g., mannose receptor) receptors expressed by macrophages, EGFR expressed by endothelial cells, and a variety of lectin receptors expressed by bacteria.

A “cell penetrating peptide” (CPP), also referred to as a “protein transduction domain” (PTD), a “membrane translocating sequence,” and a “Trojan peptide”, refers to a short peptide (e.g., from 4 to about 40 amino acids) that has the ability to translocate across a cellular membrane to gain access to the interior of a cell and to carry into the cells a variety of covalently and noncovalently conjugated cargoes, including proteins, oligonucleotides, and liposomes. They are typically highly cationic and rich in arginine and lysine amino acids. Examples of such peptides include TAT cell penetrating peptide (GRKKRRQRRRPQ) (SEQ ID NO: 4); MAP (KLALKLALKALKAALKLA) (SEQ ID NO: 5); Penetratin or Antenapedia PTD (RQIKWFQNRRMKWKK) (SEQ ID NO: 6); Penetratin-Arg: (RQIRIWFQNRRMRWRR) (SEQ ID NO: 7); antitrypsin (358-374): (CSIPPEVKFNKPFVYLI) (SEQ ID NO: 8); Temporin L: (FVQWFSKFLGRIL-NH2) (SEQ ID NO: 9); Maurocalcine: GDC(acm) (LPHLKLC) (SEQ ID NO: 10); pVEC (Cadherin-5): (LLIILRRRIRKQAHAHSK) (SEQ ID NO: 11); Calcitonin: (LGTYTQDFNKFHTFPQTAIGVGAP) (SEQ ID NO: 12); Neurturin: (GAAEAAARVYDLGLRRLRQRRRLRRERVRA) (SEQ ID NO: 13); Penetratin: (RQIKIWFQNRRMKWKKGG) (SEQ ID NO: 14); TAT-HA2 Fusion Peptide: (RRRQRRKKRGGDIMGEWGNEIFGAIAGFLG) (SEQ ID NO: 15); TAT (47-57) Y(GRKKRRQRRR) (SEQ ID NO: 16); SynB1 (RGGRLSYSRRRFSTSTGR) (SEQ ID NO: 17); SynB3 (RRLSYSRRRF) (SEQ ID NO: 18); PTD-4 (PIRRRKKLRRL) (SEQ ID NO: 19); PTD-5 (RRQRRTSKLMKR) (SEQ ID NO: 20); FHV Coat-(35-49) (RRRRNRTRRNRRRVR) (SEQ ID NO: 21); BMV Gag-(7-25) (KMTRAQRRAAARRNRWTAR) (SEQ ID NO: 22); HTLV-II Rex-(4-16) (TRRQRTRRARRNR) (SEQ ID NO: 23); HIV-1 Tat (48-60) or D-Tat (GRKKRRQRRRPPQ) (SEQ ID NO: 24); R9-Tat (GRRRRRRRRRPPQ) (SEQ ID NO: 25); Transportan (GWTLNSAGYLLGKINLKALAALAKKIL chimera) (SEQ ID NO: 26); SBP or Human P1 (MGLGLHLLVLAAALQGAWSQPKKKRKV) (SEQ ID NO: 27); FBP (GALFLGWLGAAGSTMGAWSQPKKKRKV) (SEQ ID NO: 28); MPG (ac-GALFLGFLGAAGSTMGAWSQPKKKRKV-cya (wherein cya is cysteamine)) (SEQ ID NO: 29); MPG(ANLS) (ac-GALFLGFLGAAGSTMGAW SQPKSKRKV-cya) (SEQ ID NO: 30); Pep-1 or Pep-1-Cysteamine (ac-KETWWETWWTEWSQPKKKRKV-cya) (SEQ ID NO: 31); Pep-2 (ac-KETWFET WFTEWSQPKKKRKV-cya) (SEQ ID NO: 32); Periodic sequences, Polyarginines (RxN (4<N<17) chimera) (SEQ ID NO: 33); Polylysines (KxN (4<N<17) chimera) (SEQ ID NO: 34); (RAca)6R (SEQ ID NO: 35); (RAbu)6R (SEQ ID NO: 36); (RG)6R (SEQ ID NO: 37); (RM)6R (SEQ ID NO: 38); (RT)6R (SEQ ID NO: 39); (RS)6R (SEQ ID NO: 40); R10 (SEQ ID NO: 41); (RA)6R (SEQ ID NO: 42); and R7 (SEQ ID NO: 43).

A “dye” refers to small organic molecules having a molecular weight (actual, not number average) of 2,000 Da or less or a protein which is able to emit light. Non-limiting examples of dyes include fluorophores, chemiluminescent or phosphorescent entities. For example, dyes useful in the present technology include but are not limited to cyanine dyes (e.g., Cy2, Cy3, Cy5, Cy5.5, Cy7, and sulfonated versions thereof), fluorescein isothiocyanate (FITC), ALEXA FLUOR® dyes (e.g., ALEXA FLUOR® 488, 546, or 633), DYLIGHT® dyes (e.g., DYLIGHT® 350, 405, 488, 550, 594, 633, 650, 680, 755, or 800) or fluorescent proteins such as GFP (Green Fluorescent Protein).

A “metal chelating ligand” as used herein refers to ligands that chelate metal isotopes for use in imaging. Non-limiting examples of metal chelating ligands include triazacyclononane-phosphinic acid (i.e., TRAP), 1,4,7,10-tetraazacyclododecane-1,4,7,10-tetraacetic acid (i.e., DOTA), 1,4,7-triazacyclononane-triacetic acid (i.e., NOTA), diethylenetriaminepentaacetic acid (i.e., DTPA), or chelating peptides). Thus, the metal chelating ligand may be one for use in PET or MRI.

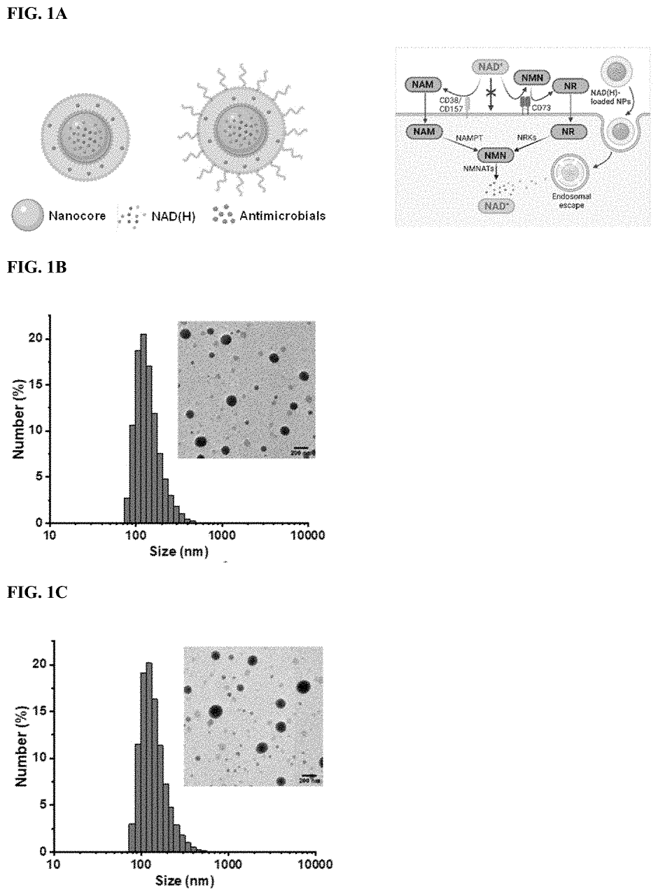

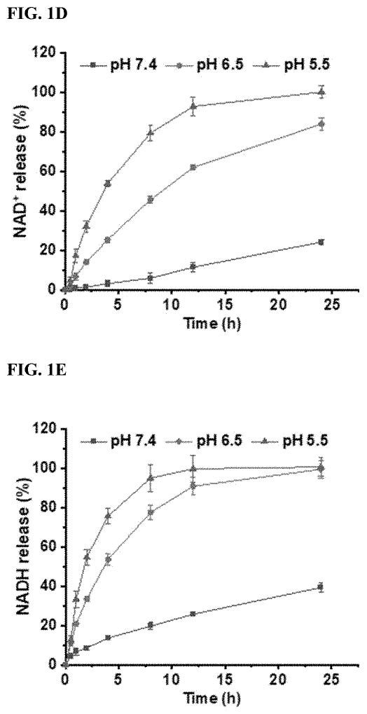

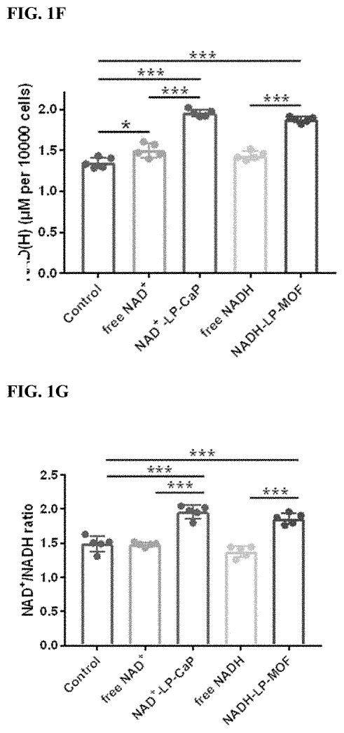

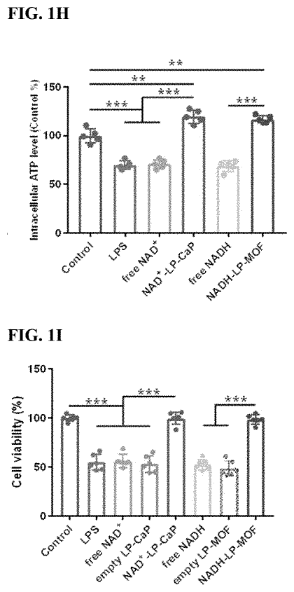

The present technology provides a nanoparticle having an inorganic core and NAD⁺ or NADH, coated with a lipid bilayer. The inorganic core may be calcium phosphate or a metal organic framework (MOF). The MOF may include a transition metal ion coordinated to a coordinating ligand, wherein the transition metal ion is selected from the group consisting of zinc, iron, zirconium, copper, and cobalt ions, and the coordinating ligand is selected from an imidazolate ligand or a carboxylate ligand. The nanoparticle may have a hydrodynamic diameter of from at least 50 nm to less than 1000 nm. FIG. 1A shows an illustrative embodiment of the NP. While not wishing to be bound by theory, it is believed that NAD(H) are loaded (i.e., are present) in the core (also referred to herein a the nanocore) of the NPs. Antimicrobials can optionally be loaded into the present NPs and may primarily be present in the core or lipid bilayer depending on the hydrophobicity of the drugs. Hydrophobic antimicrobials are believed to primarily load to the lipid bilayer, while hydrophilic antimicrobials are believed to primarily load to the core. Consistent with the Examples herein, the NAD(H)-loaded NPs are believed to be taken up by the cells via endocytosis and directly replenish cellular NAD⁺. The CaP or MOF cores are believed to dissolve in the acidic environment of the endosome, leading to endosome swelling and bursting (due to an increase in osmotic pressure) to release the entrapped payload into cytosol.

The amount of NAD⁺ or NADH included in the nanoparticle may vary, e.g., from 1 wt % to 50 wt % NAD⁺, NADH, or both. Thus, in any embodiments, the present nanoparticles may include 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 20 wt %, 25 wt %, 30 wt %, 35 wt %, 40 wt %, 45 wt %, 50 wt %, or a range between and including any two of the foregoing values, e.g., 1 wt % to 25 wt % or 2 wt % to 20 wt %, or 3 wt % to 15 wt % among others.

In any embodiments, the present nanoparticles may include lipid bilayer(s) which coat the surface of the nanoparticle in whole or in part. The present nanoparticles may include, e.g., 10 wt % to 50 wt % lipid bilayer(s). Thus, in any embodiments, the present nanoparticles may include 10 wt %, 15 wt %, 20 wt %, 25 wt %, 30 wt %, 35 wt %, 40 wt %, 45 wt %, 50 wt % lipid bilayer(s), or a range between and including any two of the foregoing values, e.g., 10 wt % to 40 wt %, 10 wt % to 30 wt %, or 15 wt % to 40 wt % lipid bilayer(s).

In any embodiments of the present NPs, any suitable lipids may be used. For example, the lipids of the lipid bilayer(s) may be selected from the group consisting of L-α-phosphatidylcholine (PC), 1,2-dioleoyl-sn-glycero-3-phosphate (DOPA), 1,2-dioleoyl-3-trimethylammonium-propane (DOTAP), 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phospho-L-serine (DOP 5), 1,2-di stearoyl-sn-glycero-3-phosphoethanolamine-N-[methoxy(polyethylene glycol)] (DSPE-PEG), cholesterol, a cell membrane extracted from a red blood cell, macrophage, neutrophil or platelet, and combinations of two or more thereof. In any embodiments of the NPs, the lipids of the lipid bilayer may include a combination of DOPA, cholesterol, and other lipids.

In any embodiments, a portion of lipids in the lipid bilayer may be conjugated to poly(ethylene glycol) (PEG). Up to 100 mol % of the lipids in the lipid bilayer may be conjugated to PEG, e.g., 1, 2, 3, 4, 5, 6, 7, 8, 10, 15, 20, 30, 40, 50, 60, 70, 80, 90, or 100 mol %, or a range between and including any two of the foregoing values. In any embodiments, the lipids of the lipid bilayer may include DSPE-PEG. The PEG (when conjugated to a lipid) includes a free terminus selected from the group consisting of OH, O—C₁₋₄ alkyl ether, NH₂, NHR, COOH, COOR, wherein R is an alkyl or alkenyl group, a dye, a targeting ligand, and a metal chelating ligand. Thus, the PEG terminus may be optionally conjugated to a dye, a targeting ligand, or a metal chelating ligand directly or through any suitable linker (e.g., with a molecular weight below about 500 Da) known in the art. The PEG may have a number average molecular weight ranging from 300 to 10000 Da. In any embodiments the PEG may have a number average molecular weight of about 300, 500, 1000, 2000, 3000, 4000, 5000, 6000, 7000, 8000, 9000, or 10000 Da or a range between and including any two of the foregoing values, e.g., 3000-7000 Da. In any embodiments, the lipid of the lipid bilayer(s) include a cell membrane extracted from a red blood cell, macrophage, neutrophil, or platelet, and combinations of two or more thereof.

In any embodiments, cell penetrating peptides, targeting ligands, and/or metal chelating agents may optionally be conjugated directly or indirectly (through any suitable linker of less than 500 Da known in the art) to lipids in the lipid bilayer.

In any embodiments, the present NPs include 40 wt %-90 wt % inorganic core, e.g., 40 wt %-90 wt % calcium phosphate or 40 wt %-90 wt % MOF. For example the present nanoparticles may include 40 wt %, 45 wt %, 50 wt %, 55 wt %, 60 wt %, 65 wt %, 70 wt %, 75 wt %, 80 wt %, 85 wt %, 90 wt % inorganic core or a combination between and including any two of the foregoing values, e.g., 40 wt %-80 wt %.

In any embodiments, the inorganic core is calcium phosphate. Calcium phosphate may be a single compound, but is more typically a mixture of two or more such compounds having, e.g., a molar ratio of Ca to P from 0.5 to 2 (e.g., 0.5, 1.2, 1.33, 1.5, 1.67, 2 or a range between and including same). In some embodiments the calcium phosphate comprises one or more of hydroxy apatite (Ca₁₀(PO₄)₆(OH)₂), Ca(H₂PO₄)₂*H₂O, Ca(H₂PO₄)2, CaHPO₄*2H₂O, Ca(HPO₄) and the like.

In any embodiments, the inorganic core of the present nanoparticles may be a MOF. As noted above the transition metal ion of the MOF may be selected from the group consisting of zinc, iron, zirconium, copper, and cobalt ions. In any embodiments the transition metal ion of the MOF may be zinc ions or iron ions. The coordinating ligand may be an imidazolate ligand or a carboxylate ligand as noted above. Imidazolate ligands are coordinating ligands that contain an imidazole group such as, e.g., imidazole itself, 2-methyl-imidazole, benzimidazole, or 5-methylbenzimidazole. Carboxylate ligands include, e.g., terephthalic acid, 2-methyl-pterphthalic acid, 2-hydroxy-terphthalic acid, and 2-amino-terphthalic acid. The imidazolate and carboxylate ions are typically but are not necessarily in their anionic forms. Those of skill in the art will recognize which ligands are suitable for use with a particular type of metal to form a metal organic framework component. By way of example only, zinc may be used with imidazolate ligands and iron may be used with carboxylate ligands, especially dicarboxylate ligands. In any embodiments, the coordinating ligand also may be selected from the group consisting of imidazole, 2-methyl-imidazole, benzimidazole, 5-methylbenzimidazole, terephthalic acid, 2-methyl-pterphthalic acid, 2-hydroxy-terphthalic acid, and 2-amino-terphthalic acid, benzene-1,3,5-tricarboxylic acid, 1,3,5-tris(4-carboxyphenyl)benzene, 2,6-naphthalenedicarboxylic acid, 4,4′,4″-s-triazine-2,4,6-triyl-tribenzoic acid, and 2,5-dihydroxyterephthalic acid. In any embodiments, the coordinating ligand is 2-methyl-imidazole. In any embodiments, the the MOF includes zinc ions and imidazolate ligands. In any embodiments, the imidazolate ligand may be selected from imidazole, 2-methyl-imidazole, benzimidazole, or 5-methylbenzimidazole. In any embodiments of the present NPs, the imidiazolate ligand may be 2-methyl-imidazole.

The present nanoparticles may include a metal organic framework component as described above. Suitable metal ions that may be employed in the metal organic framework component include zinc, iron, zirconium, copper, and cobalt ions. In any embodiments the metal ion may be zinc ion or it may be iron ion. The coordinating ligand may be an imidazolate ligand or a carboxylate ligand as noted above. Imidazolate ligands are coordinating ligands that contain an imidazole group such as, e.g., imidazole itself, 2-methyl-imidazole, benzimidazole, or 5-methylbenzimidazole. Carboxylate ligands include, e.g., terephthalic acid, 2-methyl-pterphthalic acid, 2-hydroxy-terphthalic acid, and 2-amino-terphthalic acid. The imidazolate and carboxylate ions are typically but are not necessarily in their anionic forms. Those of skill in the art will recognize which ligands are suitable for use with a particular type of metal to form a metal organic framework component. By way of example only, zinc may be used with imidazolate ligands and iron may be used with carboxylate ligands, especially dicarboxylate ligands.

As sepsis is often caused by microbial infections, in any embodiments the present NPs may further include an antimicrobial (e.g., antibiotic, antiviral, or antifungal agent). The antibiotic employed is not particularly limited but may be a gram positive-specific antibiotic, a gram negative-specific antibiotic, a broad spectrum antibiotic, or one targeted to a particular bacterial species, including drug-resistant species. In any embodiments, the antibiotic may be selected from the group consisting of rifampicin, cefepime, ciprofloxacin, minocycline, azithromycin, tigecycline, streptomycin, gentamicin, asmycin, etimicin, dacamycin, amikacin and combinations of any two or more thereof. In any embodiments, the antiviral agent may be selected from the group consisting of raltegravir, indinavir, nevirapine, sofosbuvir, amantadine, palivizumab, entecavir, lamivudine, ganciclovir, cidofovir, trifluridine, acyclovir, podofilox and combinations of any two or more thereof. In any embodiments, the antifungal agent may be selected from the group consisting of clotrimazole, econazole, micronazole, fluconazole, voriconazole, ketoconazole, terbinafine, amorolfine, isavuconazole, nystatin, echinocandin, nikkomycin Z, 5-flucytosine, tavaborole and combinations of any two or more thereof.

The present nanoparticles may have a hydrodynamic diameter ranging from at least 50 nm to less than 1000 nm. For example, the NPs may have a hydrodynamic diameter of 50, 60, 70, 80, 90, 100, 110, 130, 150, 170, 200, 250, 300, 400, 500, 600, 700, 800, 900, or less than 1000 nm or a range between and including any two of the foregoing values. Such ranges include but are not limited to NPs with a hydrodynamic diameter of 70 to 700 nm or 70 to 500 nm. Alternatively, the present technology provides NPs with a median hydrodynamic diameter, also selected from any of the foregoing values or ranges.

A pharmaceutical composition comprising a nanoparticle as described herein and a pharmaceutically acceptable carrier.

In another aspect, the present technology provides methods of treating sepsis and/or inflammation comprising administering an effective amount of any nanoparticle or pharmaceutical composition as described herein to a subject suffering from sepsis or inflammation. In any embodiments, the subject may be a mammal and may be selected from primates, dogs, cats, rodents, horses, cattle, or pigs. In any embodiments, the subject or mammal is a human. In any embodiments, the subject suffers from sepsis or inflammation caused by an infection, e.g., a bacterial, viral, or fungal infection. For example, the subject may suffer from endotoxemia and/or multidrug-resistant (MDR) pathogen-induced bacteremia. Almost any bacterial infection can cause sepsis, including but not limited to infections due to Staphylococcus aureus, Methicillin resistant, Staphylococcus aureus, Streptococcus pneumoniae, Pseudomonas aeruginosa, Enterobacter spp (including E. cloacae), Acinetobacter baumannii, Citrobacter spp (including C. freundii, C. koserii), Klebsiella spp (including K. oxytoca, K. pneumoniae), Stenotrophomonas maltophilia, Clostridium difficile, Escherichia coli, Heamophilus influenza, Tuberculosis, Vancomycin-resistant Enterococcus, Legionella pneumophila, L. longbeachae, L. feeleii, L. micdadei, and L. anisa. In some embodiments the infection is due to Staphylococcus aureus, Streptococcus peumoniae, C. difficile, E. coli, and/or P. aeruginosa, and/or drug-resistant (e.g., MRSA) or multi-drug resistant bacteria. In any embodiments, the subject suffering from sepsis or inflammation has septicemia. Viral infections may also cause sepsis, including influenza (e.g., influenza A or influenza B viruses), rhinovirus, parainfluenza virus (e.g., types 1-3), respiratory syncytial virus, adenovirus, and coronavirus (e.g, covid 19). Fungal infections, e.g, by candida or aspergillius, can also cause sepsis and/or inflammation.

In some embodiments where the sepsis or inflammation is caused by infection, the method further includes administering an effective amount of an antimicrobial (e.g., effective amount of an antibiotic, antiviral, or antifungal agent, as appropriate for, respectively, a bacterial, viral, or fungal infection) to the subject. The antimicrobials may be administered to the subject separately, simultaneously, or sequentially with the nanoparticle. Thus, the antimicrobials may be administered alone (e.g., in a separate composition, sequentially or at different times) or may be incorporated into a nanoparticle of the present technology and thus administered simultaneously with the NAD(H) in the nanoparticle. Thus, in some embodiments, the nanoparticle further comprises an effective amount of an antimicrobial for treating an infection, such as an antibacterial for treating a bacterial infection. In some embodiments, the NP further comprises an effective amount of an antiviral for treating a viral infection, and/or an effective amount of an antifungul for treating a fungal infection. In any embodiments, the antibiotic may be selected from the group consisting of rifampicin, cefepime, ciprofloxacin, minocycline, azithromycin, tigecycline, streptomycin, gentamicin, asmycin, etimicin, dacamycin, amikacin and combinations of any two or more thereof. In any embodiments, the antiviral agent may be selected from the group consisting of raltegravir, indinavir, nevirapine, sofosbuvir, amantadine, palivizumab, entecavir, lamivudine, ganciclovir, cidofovir, trifluridine, acyclovir, podofilox, paxlovid, molnupivir and combinations of any two or more thereof. In any embodiments, the antifungal agent may be selected from the group consisting of clotrimazole, econazole, micronazole, fluconazole, voriconazole, ketoconazole, terbinafine, amorolfine, isavuconazole, nystatin, echinocandin, nikkomycin Z, 5-flucytosine, tavaborole and combinations of any two or more thereof. In any embodiments, the effective amount of the nanoparticle or pharmaceutical composition comprising the nanoparticle reduces or prevents blood and/or organ damage from sepsis. For example, the organ may selected from one or more of lung, heart, liver, kidney and spleen. In any embodiments, the effective amount reduces or prevents immunosuppression caused by the sepsis. In any embodiments, the effective amount reduces or prevents inflammation-induced cell apoptosis and pyroptosis in the mammal. In any embodiments, the amount of the nanoparticle is effective to increase cellular energy supply and/or inhibit cell apoptosis and dysfunction in immune cells, thereby reducing or preventing immunosuppression and/or endothelial damage.

In another aspect, the present technology provides methods of preventing sepsis and/or inflammation and/or immunosuppression associated therewith, which comprises administering to a subject in need of such prevention (e.g., a subject with a bacterial infection) a prophylactically effective amount of any nanoparticle or composition including nanoparticles as described herein. In any embodiments, the subject has suffered a wound, e.g., a puncture wound or cut, or a surgical wound, and the prophylactically effective amount of any nanoparticle or composition including nanoparticles as described herein is effective to prevent multiple organ system failure. In any embodiments where the subject has suffered a wound, the subject may suffer from or is at risk for a secondary infection. The secondary infection may be due to any pathogen, including but not limited to any bacterium, virus, or fungus disclosed herein. In any embodiments, the secondary infection results from bacteria selected from Staphylococcus aureus, Methicillin resistant Staphylococcus aureus, Streptococcus pneumoniae, Pseudomonas aeruginosa, Enterobacter spp (including E. cloacae), Acinetobacter baumannii, Citrobacter spp (including C. freundii, C. koserii), Klebsiella spp (including K. oxytoca, K. pneumoniae), Stenotrophomonas maltophilia, Clostridium difficile, Escherichia coli, Heamophilus influenza, Tuberculosis, Vancomycin-resistant Enterococcus, Legionella pneumophila, L. longbeachae, L. feeleii, L. micdadei, and L. anisa. In some such embodiments, the secondary infection is a P. aeruginosa secondary infection.

In yet another aspect, the present technology provides methods of decreasing a level of TNF-α or IL-6 in a cell comprising administering an effective amount of a nanoparticle as described herein to the cell. The cell may be in vitro or in vivo. The present technology may further provide methods of decreasing a level of TNF-α or IL-6 in animal subject, comprising administering an effective amount of a nanoparticle as described herein to the subject. In any embodiments, the effective amount may be effective to decrease the level of TNF-α or IL-6 in the blood and or tissue. In any embodiments, the methods include decreasing elevated a level of TNF-α and/or IL-6 resulting from sepsis. In any embodiments, the methods include decreasing elevated a level of TNF-α and/or IL-6 resulting from a microbial infection (including any disclosed herein), cancer, autoimmune conditions and/or mongenic disorders

The compositions described herein can be formulated for various routes of administration, for example, by parenteral, intravitreal, intrathecal, intracerebroventricular, rectal, nasal, vaginal administration, direct injection into the target organ, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not interfere with formation of the nanoparticles described herein. Injectable forms may be prepared with acceptable solvents or vehicles including, but not limited to sterilized water, phosphate buffer solution, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Exemplary carriers and excipients may include but are not limited to USP sterile water, saline, buffers (e.g., phosphate, bicarbonate, etc.), tonicity agents (e.g., glycerol),

Specific dosages may be adjusted depending on conditions of disease, the age, body weight, general health conditions, sex, and diet of the subject, dose intervals, administration routes, excretion rate, and combinations of drug conjugates. Any of the above dosage forms containing effective amounts are well within the bounds of routine experimentation and therefore, well within the scope of the instant present technology. By way of example only, such dosages may be used to administer effective amounts of the present nanoparticles to the patient and may include 0.1, 0.5, 1.0, 2.0, 3.0, 4.0, 5.0, 10, 15, 20, 25, 30, 40, 50, 60, 70, 80, 90, 100 mg/kg or a range between and including any two of the forgoing values such as 0.1 to 100 mg/kg. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies. More frequent administration is also possible. Alternatively, the compositions may be administered once per day on 2, 3, 4, 5, 6 or 7 consecutive days. A complete regimen may thus be completed in only a few days or over the course of 1, 2, 3, 4 or more weeks.

The examples herein are provided to illustrate advantages of the present technology and to further assist a person of ordinary skill in the art with preparing or using the nanoparticles compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

### Materials and General Procedures

Materials. β-Nicotinamide adenine dinucleotide (NAD⁺), β-Nicotinamide adenine dinucleotide, reduced disodium salt (NADH), calcium chloride (CaCl₂), disodium hydrogen phosphate (Na₂HPO₄), zinc nitrate hexahydrate, 2-methylimidazole, and lipopolysaccharides (LPS from Escherichia coli O111:B4) were purchased from Sigma-Aldrich (St. Louis, Mo., USA). L-α-phosphatidylcholine (Soy PC) and dioleoylphosphatidic acid (DOPA) were purchased from Avanti Polar Lipids (Alabaster, Ala., USA). Other reagents and solvents were purchased from Thermo Fisher Scientific (Fitchburg, Wis., USA) and used as received unless otherwise stated.

Characterization. The hydrodynamic size and zeta potential of the NPs were measured by dynamic light scattering (DLS, ZetaSizer Nano ZS90, Malvern Instruments, USA) at a concentration of 0.1 mg/mL. The morphologies of the NPs were visualized by transmission electron microscopy (TEM, Philips CM200 Ultra Twin) with phosphotungstic acid staining. To evaluate the drug loading efficiency and loading content of the NAD(H)-loaded NPs, the NPs were dissolved in 0.01 M HCl to release NAD(H). The pH of the solutions was adjusted to neutral, and the NAD(H) concentration was determined using an Amplite™ Colorimetric Total NAD and NADH Assay Kit (AAT Bioquest, Sunnyvale, Calif., USA). The loading efficiency and loading content in percentages were calculated according to the following equations: Loading efficiency (%)=(weight of encapsulated drug/total weight of drug added)×100%. Loading content (%)=weight of encapsulated drug/total weight of nanoparticles×100%.

In vitro release kinetics of NAD(H) and Rif from the nanoparticles under different pH conditions was studied by a dialysis method. The nanoparticles (3 mL, containing 6 mg NAD(H) or 3 mg Rif) were sealed in dialysis bags (molecular weight cut-off of 3500 Da) and immersed in 40 mL buffer with different pH (pH 5.5, 6.5, 7.4). The dialysis systems were incubated in a 37° C. shaker and 100 μL samples outside the dialysis bag were collected at different time intervals for NAD(H) quantification. An equal volume of pre-warmed fresh medium was added back to the release medium.

Cell Culture. Bone marrow-derived macrophages (BMDMs) were isolated and cultured as described previously (Sendler, M. et al. Cathepsin B-mediated activation of trypsinogen in endocytosing macrophages increases severity of pancreatitis in mice. Gastroenterology 154, 704-718. e710 (2018)). Mouse neutrophils were isolated by Percoll-based density gradient centrifugation and used freshly (see Martinez-Garcia, J. J. et al. P2X7 receptor induces mitochondrial failure in monocytes and compromises NLRP3 inflammasome activation during sepsis. Nature communications 10, 1-14 (2019). Human umbilical vein endothelial cells (HUVECs) were cultured in endothelial basal medium (ATCC, USA) supplemented with endothelial cell growth kit-VEGF (ATCC) at 37° C. in 5% CO₂ atmosphere. The passage number of HUVECs used in this study was between 4 and 7. Murine macrophage cell line RAW 264.7 and human embryonic kidney cell line HEK 293 were maintained in RPMI-1640 (Gibco, USA) and DMEM media, respectively.

Cellular NAD(H) level. BMDMs were seeded into a 96-well plate (7×10⁴ cells/well) and cultured overnight. Thereafter, the cells were treated with 10 μM free NAD(H) or NAD(H)-loaded NPs with an equivalent amount of NAD(H) (10 μM) and incubated with LPS (100 ng/mL), H₂O₂ (0.5 mM), or FK866 (20 nM) for 12 h. Subsequently, the cells were washed with PBS for 3 times and lysed using 50 μL lysis buffer. The cellular NAD(H) level was assessed using the Total NAD and NADH Assay Kit. To further evaluate the NAD⁺/NADH ratio after different treatments, BMDMs were seeded in a 24-well plate (1×10⁶ cells/well) and cultured overnight. Subsequently, the cells were subjected to various treatment conditions as stated above for 12 h, and then lysed. The NAD⁺/NADH ratio was measured by an Amplite™ Colorimetric NAD/NADH Ratio Assay Kit (AAT Bioquest, Sunnyvale, Calif., USA) according to the manufacturer's instructions.

Cell viability and ATP level assays. BMDMs were seeded into a 96-well plate (2×10⁴ cells/well) and cultured overnight. Subsequently, cells were treated with free NAD(H), empty NPs, or NAD(H)-loaded NPs, together with LPS (100 ng/mL) or NAD⁺ synthesis inhibitor (FK866, 20 nM) for 48 h, or H₂O₂ (0.5 mM) for 12 h. Cell viability was measured using a standard MTT assay, and cellular ATP levels were tested using a luminescent ATP detection kit (Abcam, USA). Data were collected using a GloMax-Multi Microplate Multimode Reader (Promega, Wis., USA).

Cell pyroptosis assays. BMDMs were cultured overnight in a 96-well plate (2×10⁴ cells/well) and primed with LPS (100 ng/mL) for 3 h as the signal 1 of the NLRP3 inflammasome pathway. The cells were then stimulated with either ATP (2.5 mM, signal 2 for the canonical inflammasome pathway) for another 1 h, or lipofectamine 2000 transfected LPS (lipoLPS, 100 ng LPS/well, signal 2 for the non-canonical inflammasome pathway) in serum free media for 3 h (Hagar, J. A., Powell, D. A., Aachoui, Y., Ernst, R. K. & Miao, E. A. Cytoplasmic LPS activates caspase-11: implications in TLR4-independent endotoxic shock. Science 341, 1250-1253 (2013); Kayagaki, N. et al. Caspase-11 cleaves gasdermin D for non-canonical inflammasome signalling. Nature 526, 666-671 (2015). Cells were incubated with different treatments (free NAD(H), empty NPs, and NAD(H)-loaded NPs with an equivalent NAD(H) dose of 10 μM) together with either signal 1, signal 2, or both. Twelve hours after the treatment, cells were stained with FAM-fluorochrome inhibitor of caspases (FLICA) probe 5-FAM-YVAD-FMK (SEQ ID NO: 44) (AAT Bioquest, Sunnyvale, Calif., USA) to monitor caspase1 activation. The IL-10 and LDH release in the cell culture medium were tested by ELISA (R&D Systems, USA) and LDH assay kit (GBiosciences, St Louis, Mo., USA), respectively. All the assays above were performed according to the manufacturer's protocol.

Measurement of inflammation related cytokine levels. BMDMs were seeded into a 96-well plate (2×10⁴ cells/well) and treated with LPS (100 ng/mL) together with free NAD(H), empty NPs, or NAD(H)-loaded NPs with an equivalent NAD(H) dose of 10 μM. Twelve hours after the treatment, the cell culture media were collected to analyze the cytokine production by ELISA kits supplied by R&D Systems according to the manufacturer's instructions. Data were collected by monitoring the difference between the absorbance at 450 nm and 570 nm using the Microplate Reader.

Apoptosis-associated speck-like protein (ASC) speck formation. BMDMs seeded into confocal dishes (3×10⁵ cells/dish) overnight were primed with LPS (100 ng/mL, 3 h) and then stimulated with either ATP (2.5 mM) for 1 h or lipoLPS (1 μg LPS/dish, in serum free media) for 3 h. Different treatments, including free NAD(H), empty NPs, and NAD(H)-loaded NPs with an equivalent NAD(H) dose of 10 μM, were given together with the stimuli. The cells were fixed with freshly prepared 4% paraformaldehyde PBS solution for 20 min at room temperature and then permeabilized with 0.5% Triton X-100 for 15 min. After the cells were blocked with 10% normal goat serum at room temperature for 1 h, they were incubated with Alexa fluor 488-labeled anti-ASC antibody (Santa Cruz) for 2 h, and then stained with DAPI for 15 min at room temperature. ASC speck was then observed by CLSM.

NF-κB p65 nuclear translocation. Immunofluorescent staining was used to determine NF-κB nuclear translocation. BMDMs seeded in confocal dishes (3×10⁵ cells/dish) overnight were pretreated with free NAD(H), empty NPs, or NAD(H)-loaded NPs (with an equivalent NAD(H) dose of 10 μM) for 5 h, and then stimulated with LPS (100 ng/mL) for 1 h. The cells were fixed with freshly prepared 4% paraformaldehyde PBS solution for 20 min at room temperature and then permeabilized with 0.5% Triton X-100 for 15 min. After the cells were blocked with 10% normal goat serum for 1 h at room temperature, they were incubated with Alexa fluor 488-labeled anti-NF-κB p65 antibody (F-6, Santa Cruz) for 2 h, and then stained with DAPI for 15 min at room temperature. p65 nuclear translocation was observed by CLSM.

Apoptosis analysis. BMDMs or HUVECs were seeded into 96-well plates (2×10⁴ cells/well) and cultured overnight. BMDMs were then incubated with LPS (100 ng/mL) or FK866 (20 nM) for 48 h, or H₂O₂ (0.5 mM) for 12 h. HUVECs were incubated with LPS (100 ng/mL), FK866 (20 nM), or TNF-α (80 ng/mL) for 48 h. Different treatments, including free NAD(H), empty NPs, and NAD(H)-loaded NPs (with an equivalent NAD(H) dose of 10 μM), were added together with the stimuli. Thereafter, the cells were stained with Annexin V and propidium iodide (PI) (BD Biosciences, USA) in the Annexin V binding solution at room temperature for 20 min. Apoptotic cells were quantified using flow cytometry. The data were analyzed using FlowJo software (Tree Star). Q1 (FITC−, PI−) indicates viable cells, Q2 (FITC+, PI−) indicates cells in early-stage apoptosis, Q3 (FITC+, PI+) indicates cells in late-stage apoptosis, and Q4 (FITC−, PI−) indicates dead cells. The cells in Q2 and Q3 were defined as apoptotic cells.

Visualization of tight junctions. HUVECs monolayers were treated with free NAD(H), empty NPs, or NAD(H)-loaded NPs, together with LPS (4 μg/mL) or TNF-α (100 ng/mL) for 24 h. The cells were fixed with 4% paraformaldehyde PBS solution and permeabilized with 0.5% Triton X-100. After the cells were blocked with 10% normal goat serum for 1 h, they were incubated with Alexa fluor 488-labeled VE-cadherin antibody (Santa Cruz) for 2 h, stained with DAPI for 15 min at room temperature, and then observed by CLSM.

Mitochondrial membrane potentials (ΔΨm) assay. JC-1 fluorescence probe was used to evaluate the mitochondrial depolarization in BMDM. Briefly, cells cultured in confocal dishes were treated with free NAD(H), empty NPs, and NAD(H)-loaded NPs, and incubated with LPS (100 ng/mL) or FK866 (20 nM) for 24 h, or H₂O₂ (0.5 mM) for 6 h. Then, the cell medium was discarded, and serum-free medium containing JC-1 probe (5 μg/mL, AdipoGen Life Sciences, San Diego, Calif., USA) was added. The cells were incubated at 37° C. for 30 min. Subsequently, cells were stained with DAPI and observed by CLSM.

Quantitative real-time PCR (qRT-PCR) analysis. BMDMs were seeded in 24-well plates (2×10⁵ cells/well) and allowed to grow for 12 h. Then, cells were primed with LPS (100 ng/mL, 3 h) and stimulated with either ATP (2.5 mM) for 1 h or lipoLPS (0.5 μg LPS/well, in serum free media) for 3 h. Free NAD(H), empty NPs, or NAD(H)-loaded NPs with an equivalent NAD(H) dose of 10 μM were incubated together with the stimuli. Total cell RNA was extracted using TRIzol reagent (Invitrogen) according to the manufacturer's instructions. RNA was converted to cDNAs by an iScript Reverse Transcription Supermix for qRT-PCR (BIO-RAD). qRT-PCR was performed with an iTaq Universal SYBR Green Supermix (BIO-RAD)⁵. The primers used for qRT-PCR are listed in table 1.

TABLE 1
Primers used for qRT-PCR

| Genes | Forward (5′-3′) | SEQ ID NO: | Reverse (5′-3′) | SEQ ID NO: |
|---|---|---|---|---|
| cox-2 | GTACCGCAAACGCTTCTCCCTG | 45 | CCTCCAAAGGTGCTCGGCTTCCA | 57 |
| nlrp3 | TGGATGGGTTTGCTGGGAT | 46 | CTGCGTGTAGCGACTGTTGAG | 58 |
| inos | AGGCTCCTCACGCTTGGGTCTTG | 47 | CTGCGGGGAGCCATTTTGGTG | 59 |
| caspas | GCCACTTGCCAGGTCTACGAG | 48 | AGGCCTGCACAATGATGACTTT | 60 |
| p65 | ACTGCCGGGATGGCTACTAT | 49 | TCTGGATTCGCTGGCTAATGG | 61 |
| p50 | CTGGCAGCTCTTCTCAAAGC | 50 | TCCAGGTCATAGAGAGGCTCA | 62 |
| pro- | TGGGCCTCAAAGGAAAGA | 51 | GGTGCTGATGTACCAGTT | 63 |
| caspas | ACAAGGCACGGGACCTATG | 52 | TCCCAGTCAGTCCTGGAAATG | 64 |
| ctsb | AGACCTGCTTACTTGCTGTG | 53 | GGAGGGATGGTGTATGGTAAG | 65 |
| gsdmd | CCATCGGCCTTTGAGAAAGTG | 54 | ACACATGAATAACGGGGTTTCC | 66 |
| drp1 | CGGTTCCCTAAACTTCACGA | 55 | GCACCATTTCATTTGTCACG | 67 |
| caspas | GGTATTGAGACAGACAGTGG | 56 | CATGGGATCTGTTTCTTTGC | 68 |

Animals and ethics. All animal experiments were performed under the Guide for the Care and Use of Laboratory Animals (National Institutes of Health) and protocol (M006127) approved by the Institutional Animal Care and Use Committee (IACUC) at the University of Wisconsin. BALB/c (8-12 weeks, The Jackson Laboratory) mice were used for the in vivo experiments.

In vivo biodistribution study. Mice were intravenously injected with LPS (7.5 mg/kg) to induce endotoxemia (sepsis). Twelve hours later, Cy5.5-loaded LP-CaP (0.5 mg/kg) was injected to septic or healthy mice via tail vein. Mice (n=3) were sacrificed 4 h and 24 h after nanoparticle injection. Major organs including thigh muscle (M), heart (H), lungs (Lu), spleen (S), kidneys (K) and liver (L) were collected for IVIS analysis with an excitation band-pass filter at 676 nm and emission at 705 nm. For the evaluation of ATP levels in different organs, mice were challenged with LPS (15 mg/kg, IV) and treated with free NAD⁺ (20 mg/kg), or an equivalent dose of LP-CaP and NAD⁺-LP-CaP (20 mg/kg of NAD⁺) after 1 h. The mice were sacrificed 24 h after LPS administration, and different organs or tissues were collected, weighed, homogenized, and lysed for ATP quantification using a luminescent ATP detection kit.

Survival study in the endotoxemia (LPS-induced sepsis) mouse model. Mice were intravenously injected with LPS (15 mg/kg) through the tail vein. After one hour, mice were grouped randomly, and intravenously injected with various formulations including PBS, free NAD⁺, empty LP-CaP, NAD⁺-LP-CaP, free NADH, empty LP-MOF, and NADH-LP-MOF (with an equivalent NAD(H) dose of 20 mg/kg). Mice were monitored for their behavioral profile over the duration of the experiments according to the animal protocol guidelines. The survival and body weight of the mice were recorded over time and presented using Kaplan—Meier curves (n=10).

Quantification of pro-inflammatory cytokine and inflammation-related gene expression in endotoxemia (LPS-induced septic) mice. LPS-induced septic mice (7.5 mg/kg, IV) were intravenously treated with PBS, free NAD(H), empty NPs, and NAD(H)-loaded NPs with an equivalent NAD(H) dose of 20 mg/kg. Six hours after LPS challenge, blood was collected from orbital sinus and centrifuged at 300 g for 10 min to separate blood cells and plasma. The pro-inflammatory cytokine levels (TNF-α and IL-1(3) in the plasma were determined by ELISA kits. The white blood cells were collected for qRT-PCR analysis (n=6).

Lung vascular permeability measurements. Endotoxemia (LPS-induced septic) mice (7.5 mg/kg, IV) were intravenously treated with PBS, free NAD(H), empty NPs, and NAD(H)-loaded NPs with an equivalent NAD(H) dose of 20 mg/kg 1 h after LPS inoculation. Twelve hours after LPS challenge, 150 μL Evans blue-albumin (1% Evans blue dye and 4% albumin in PBS) was IV injected and allowed to circulate in the blood vessels for 1 h. Mice were scarified and lung tissues were collected and homogenized in 1 mL PBS. Evans blue was then extracted in 2 mL formamide, 60° C. overnight. Evans blue concentration in the lung homogenate supernatants was quantified by the spectrophotometric method at absorbance of 620 nm and 740 nm to determine the dye leakage (n=6) (Henry, B. D. et al. Engineered liposomes sequester bacterial exotoxins and protect from severe invasive infections in mice. Nature biotechnology 33, 81-88 (2015)).

Flow cytometry. Endotoxemia (LPS-induced septic) mice (7.5 mg/kg, IV) were intravenously treated with PBS, free NAD(H), empty NPs, and NAD(H)-loaded NPs with an equivalent NAD(H) dose of 20 mg/kg 1 h after LPS inoculation. Twenty four hours after LPS challenge, mice were sacrificed and their blood, lungs and spleen were collected (n=5). Immune cells in the lungs and spleen were isolated by squeezing through a 70-μm nylon mesh to create the single cell suspension. Red blood cells were lysed using ACK buffer (BioWhittaker) and cells were washed with PBS twice. The isolated cells were blocked with anti-mouse CD16/CD32 antibody (eBioscience) for 15 min and stained with FLICA or apoptosis antibody panels (Table 2). Flow cytometry was performed on an Attune N×T cytometer using standard procedures, and data was analyzed using FlowJo software (Tree Star). Gating strategies were set based on fluorescence minus one samples.

TABLE 2
Antibodies and markers used for flow cytometry.

| | | | |
|---|---|---|---|
| Antibody/Marker | Fluorophore | Clone | Company |

Apoptosis Panel

| | | | |
|---|---|---|---|
| CD45 | BB515 | 30-F11 | BD Biosciences |
| CD11b | PE-Cy5 | M1/70 | Tonbo Biosciences |
| Ly6C | APC | HK1.4 | Biolegend |
| Ly6G | Violet Fluor 500 | 1A8 | Tonbo Biosciences |
| CD3 | PE-Cy7 | 145-2C11 | Tonbo Biosciences |
| CD8 | Alexa Fluor 700 | 53-6.7 | Biolegend |
| CD4 | Violet Fluor 450 | RM4-5 | Tonbo Biosciences |
| Annexin V | PE | | Tonbo Biosciences |
| 7AAD | | | Tonbo Biosciences |

FLICA Panel

| | | | |
|---|---|---|---|
| CD45 | PE | 30-F11 | Tonbo Biosciences |
| CD11b | PE-Cy5 | M1/70 | Tonbo Biosciences |
| Ly6C | APC | HK1.4 | Biolegend |
| Ly6G | Violet Fluor 500 | 1A8 | Tonbo Biosciences |
| CD3 | PE-Cy7 | 145-2C11 | Tonbo Biosciences |
| CD8 | Alexa Fluor 700 | 53-6.7 | Biolegend |
| CD4 | Violet Fluor 450 | RM4-5 | Tonbo Biosciences |
| Caspase-1 | FAM-YVAD- | | AAT Bioquest |
| | FMK (SEQ ID | | |
| | NO: 44) | | |

Cecal ligation and puncture (CLP) and P. aeruginosa secondary infection model. Mice were anesthetized with isoflurane. Thereafter, the abdominal cavity was opened with a midline incision to expose the cecum. The distal 50% of the cecum was ligated with a 6-0 silk suture and perforated with a 19-gauge needle twice. Subsequently, a small amount of feces was squeezed out and spread around the cecum. The cecum was relocated into the peritoneal cavity, and the laparotomy site was closed, followed by subcutaneous injection of 1 mL resuscitative prewarmed sterile saline (Dawulieti, J. et al. Treatment of severe sepsis with nanoparticulate cell-free DNA scavengers. Science advances 6, eaay7148 (2020)). This level of injury was utilized to create a prolonged infection that impaired immune system and induced approximate 30% mortality. Two injections of free NAD⁺ (20 mg/kg per injection), LP-CaP, or NAD⁺-LP-CaP (an equivalent dose of NAD⁺20 mg/kg per injection) were given through tail vein 6 h and 24 h after the surgery. At day 3 post CLP, the surviving mice were anesthetized and infected with P. aeruginosa (ATCC 27853 1×10⁸ CFU in 50 μL PBS solution) through intratracheal injection, to mimic nosocomial infections. A rodent intubation stand with 45 degree angle and a syringe fitted with a polyethylene tubing PE-10 were used for the intratracheal instillation. After anesthesia, the mice were suspended by their incisors in the supine position on the intubating platform. Curved blunt-ended forceps were used to carefully open the mouth and grasp the tongue, allowing the PE-10 tubing to be inserted into the trachea to instill the samples. The mice were maintained in the supine position on the intubating platform for at least 30 seconds. Thereafter, the mice were placed prone on a heating pad for recovery. Mice survival and body weight were recorded over time and presented using Kaplan—Meier curves (n=14). A Sham group without CLP and a CLP group without P. aeruginosa challenge were applied as control groups. The CLP mice were monitored twice daily and their conditions were evaluated using a sepsis scoring sheet. The moribund animals with their Murine Sepsis Score (MSS) above 21 were euthanized before spontaneous death.

MRSA and P. aeruginosa-induced polymicrobial blood infection model. Mice were infected with 0.1 mL bacterial suspension (mixed MRSA ATCC 33591 and P. aeruginosa with 5×10′ CFU for each pathogen) through tail vein injection. Six hours after infection, the mice were grouped randomly, and intravenously injected with various formulations including PBS, NAD⁺-LP-CaP, free Rif, Rif-LP-CaP, and NAD⁺-Rif-LP-CaP (20 mg/kg NAD⁺ and 10 mg/kg Rif) (n=10). The survival and body weight of the mice was recorded over time.

CFU detection. In the MRSA and P. aeruginosa-induced polymicrobial blood infection model, twelve hours after bacteria inoculation, the animals were euthanized by CO₂ asphyxiation. Blood and major organs (i.e., liver, spleen, lungs, kidneys) were collected and homogenized in 3 mL PBS. The CFU in the blood and infected organs were determined by serial dilution and plate counting.

Histopathological analysis. Organ injury in the polymicrobial infection model was evaluated by blood biochemistry and histological analysis. Twenty-four hours after bacterial inoculation, the animals with different treatments were sacrificed and their blood was collected for blood biochemistry tests, including aspartate transaminase (AST), alanine transaminase (ALT), alkaline phosphatase (ALP), blood urea nitrogen (BUN), and creatine kinase (CRE). Major organs including heart, liver, spleen, lungs, and kidneys were harvested and sectioned. H&E staining was carried out for histological examinations.

A TUNEL assay was performed on tissue sections using a chromogenic kit (ABP Biosciences, Beltsville, Md., USA). Tissue sections were incubated with terminal deoxynucleotidyl transferase (TdT) reaction cocktail at 37° C. for 2 h, washed three times with PBS, and then incubated with horseradish peroxidase-streptavidin staining solution at 37° C. for 30 min. Thereafter, color development was performed using diaminobenzidine (DAB) substrate. The tissue sections were examined under an optical microscope (Olympus Plaza, Tokyo, Japan).

Statistical Analysis. Results are presented as mean±standard deviation (SD). One-way analysis of variance (ANOVA) with Tukey's multiple comparisons was used to determine the difference between independent groups. Statistical analyses were conducted using GraphPad Prism software version 6.

### Example 1—Preparation of NAD⁺-Lipid-Calcium Phosphate Nanoparticles (NAD⁺-LP-CaP NPs)

NAD⁺-LP-CaP was prepared via a water-in-oil reverse micro-emulsion method followed by thin-film hydration. DOPA, capable of forming strong interactions with cations at interfaces, was used to form the inner leaflet of the lipid bilayer to coat the CaP core, while PC was used to form the outer leaflet of the lipid bilayer to stabilize the NP. Briefly, two water-in-oil reverse microemulsions (25 mL) dispersed in a cyclohexane/Igepal CO-520 (71/29, v/v) oil phase were prepared. Emulsion 1 contained 500 μL CaCl₂ solution (2.5 M) with 1 mg NAD⁺, while emulsion 2 contained 500 μL Na₂HPO₄ solution (25 mM, pH 9) with 3 mg DOPA. The two emulsions were mixed and stirred for 30 min. Ethanol (50 mL) was added to remove the oil phase and the NP was pelleted by centrifugation at 10,000 g for 20 min. After being extensively washed by ethanol for three times, the NP together with Soy PC (3 mg) and cholesterol (0.3 mg) was dissolved in chloroform. After chloroform was removed by a rotary evaporator, the lipid film was then rehydrated in 500 μL Tris-HCl buffer (10 mM, pH 7.4) to obtain NAD⁺-LP-CaP. NAD⁺ and Rif co-loaded nanoparticles (NAD⁺-Rif-LP-CaP) with Rif encapsulated in the lipid coating layer were prepared with the aforementioned processes by adding Rif together with Soy PC and cholesterol.

### Example 2—Preparation of NADH-Lipid-Metal Organic Framework Nanoparticles (NADH-LP-MOF)

The NADH-LP-MOF NP was prepared by mixing zinc nitrate, 2-methylimidazole, and NADH in water under ultrasonication to yield the ZIF-8 core, which was subsequently stabilized by lipid coating via an extrusion process. NADH (6.6 mg) and zinc nitrate hexahydrate (18.6 mg) were dissolved in 35 mL DI water. 2-Methylimidazole (166 mg, dissolved in 10 mL DI water) was added to the NADH solution. The resultant mixture was vortexed for 10 s and then kept still for 5 min to allow MOF growth. The MOF nanoparticle was then pelleted through centrifugation at 10,000 g for 45 min, redispersed in 1.5 mL water and ultrasonicated for 30 times. The MOF nanoparticle was mixed with a liposome solution (composed of Soy PC and cholesterol, 10:1 w/w, 40 mg/mL) and extruded through a 0.4 μm polycarbonate porous membrane using an Avanti mini extruder to obtain NADH-LP-MOF.

### Example 3—Characterization of NAD(H) NPs

Both NAD⁺-LP-CaP and NADH-LP-MOF had hydrodynamic diameters around 150 nm analyzed by dynamic light scattering (DLS), and they both displayed spherical morphology as shown in the transmission electron microscopy (TEM) images (FIGS. 1B, 1C). The loading efficiencies of NAD⁺-LP-CaP and NADH-LP-MOF were 68.8% and 62.9%, respectively, and their loading contents were 12.4% and 5.2%, respectively. The NPs with pH-sensitive CaP and MOF cores demonstrated pH-responsive NAD(H) release behaviors, which is desirable for this particular application (FIGS. 1D, 1E). The NPs' higher stability in the physiological environment prevented premature drug release in the bloodstream and protected the loaded NAD(H) from enzyme degradation. Once taken up by the cells, the NPs can quickly release their payloads and facilitate their escape from endosome to cytosol via a proton sponge effect, thus protecting NAD(H) from low pH-induced degradation.

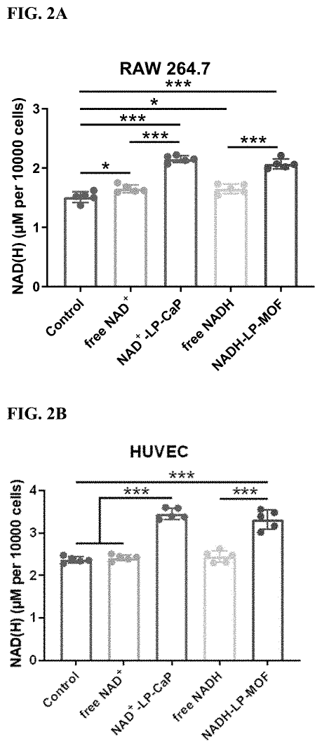

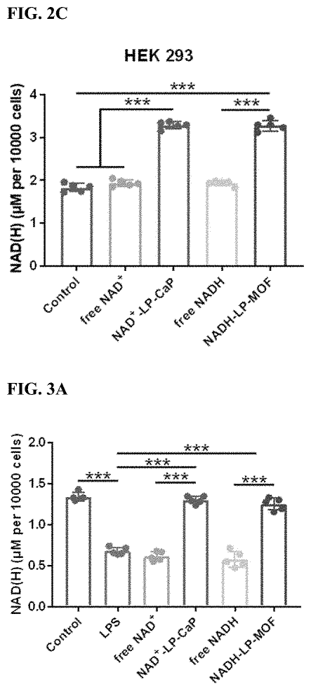

The ability of NAD(H)-loaded NPs to effectively replenish the cellular NAD(H) pool in multiple types of cells was investigated, including murine bone marrow-derived macrophage (BMDM), murine macrophage cell line RAW 264.7, human umbilical vein endothelial cell (HUVEC), and human embryonic kidney (HEK) 293 cell (FIG. 1F; FIG. 2). Both NAD⁺-LP-CaP and NADH-LP-MOF significantly enhanced the intracellular NAD(H) levels in each of the four cell types investigated, ranging from 41% to 80%. In contrast, free NAD(H) showed a much weaker effect or no effect. While the NAD(H) level in the cells is quite high (˜300 μM), a much lower concentration of the NPs in the cell culture media (containing 10 μM NAD(H)) effectively enhanced intracellular NAD(H) level, suggesting their superior capacity in replenishing cellular NAD⁺ pool.

The cellular NAD⁺/NADH ratio is an important factor reflecting cell metabolism and redox status. Thus, the effect of the two types of NPs on the NAD⁺/NADH ratio in BMDM was investigated. Despite delivering different forms of NAD, as shown in FIG. 1G, both NAD⁺-LP-CaP and NADH-LP-MOF increased the NAD⁺/NADH ratio. This may be due to the cells' reported ability to interconvert the extra NADH (delivered by NADH-LP-MOF in this case) to NAD⁺ through a set of redox systems such as the TCA cycle, complex I of the electron transport chain, and various cytosolic dehydrogenases, to maintain the NAD⁺/NADH balance.

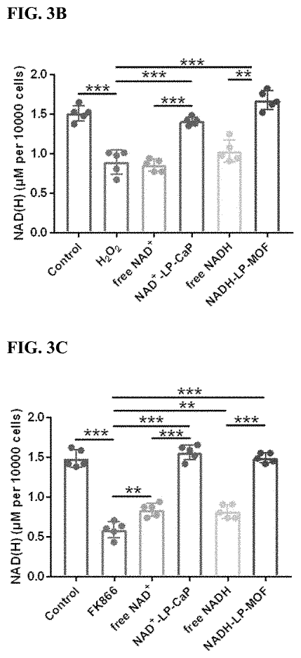

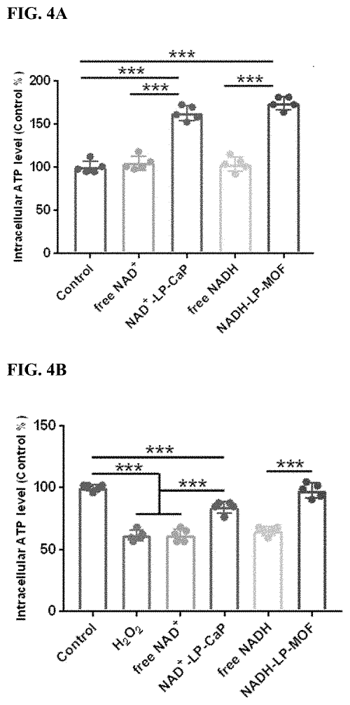

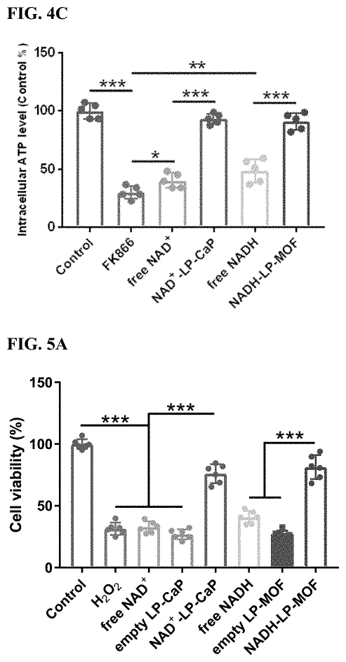

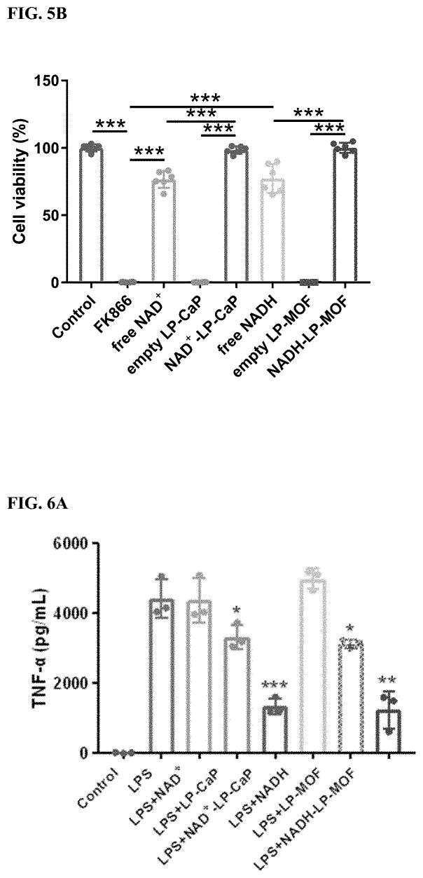

Inflammation and the resultant oxidative stress can trigger a unique metabolic status that consumes cellular NAD(H) quickly due to elevated PARP, sirtuins activations, and CD38 expression. The cytotoxicity of the NAD(H)-loaded NPs on BMDMs was evaluated and is shown in FIG. 35. Both NAD⁺-LP-CaP and NADH-LP-MOF showed good biocompatibility at an NAD(H) equivalent concentration of 10 μM. The NAD(H)-loaded NPs effectively replenished NAD(H) pool in the inflammatory cells and prevented NAD(H) loss induced by inflammation (LPS), oxidative stress (hydrogen peroxide) and NAD⁺ depletion (NAMPT inhibitor FK866), while free NAD(H) did not show much benefit (FIG. 3). NAD⁺ is a key coenzyme participating in most of the energy-producing pathways including glycolysis and oxidative phosphorylation, so maintaining NAD⁺ homeostasis may be important in preventing energy depletion and the resultant cell damage during inflammation. The NAD(H)-loaded NPs helped to enhance the cellular ATP level in healthy cells. They also improved the ATP supply as well as cell viability for cells undergoing inflammation, oxidative stress or NAD⁺ depletion (FIGS. 1H, 1I; FIGS. 4-5).

### Example 4—NAD(H)-Loaded NPs Prevents Inflammation Induced Cell Death and Dysfunction

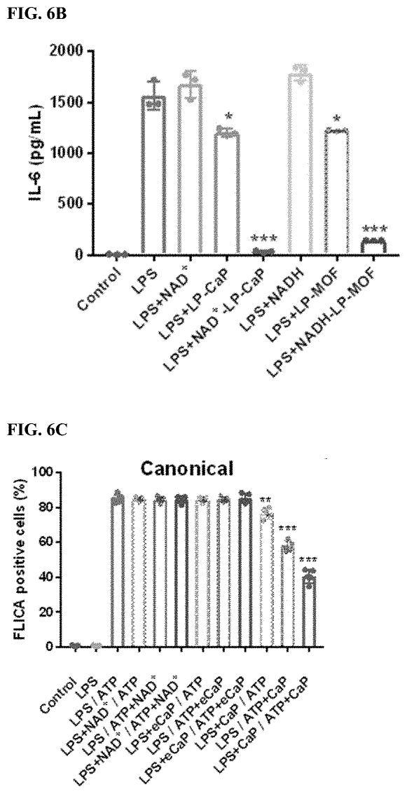

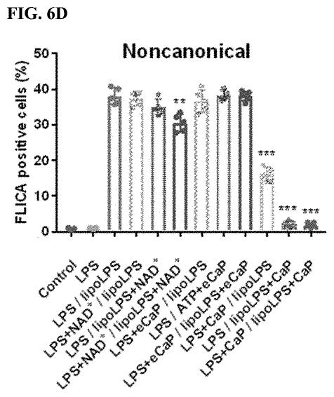

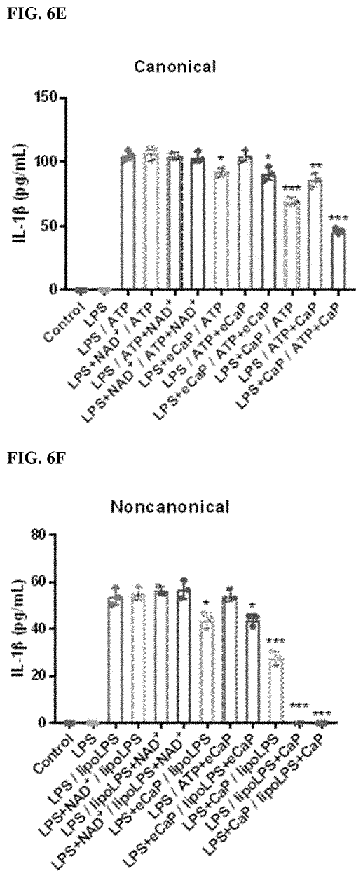

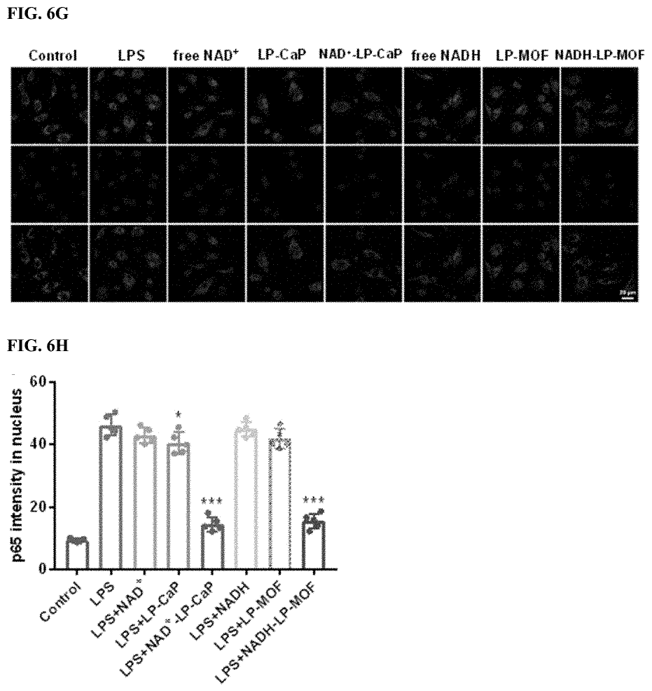

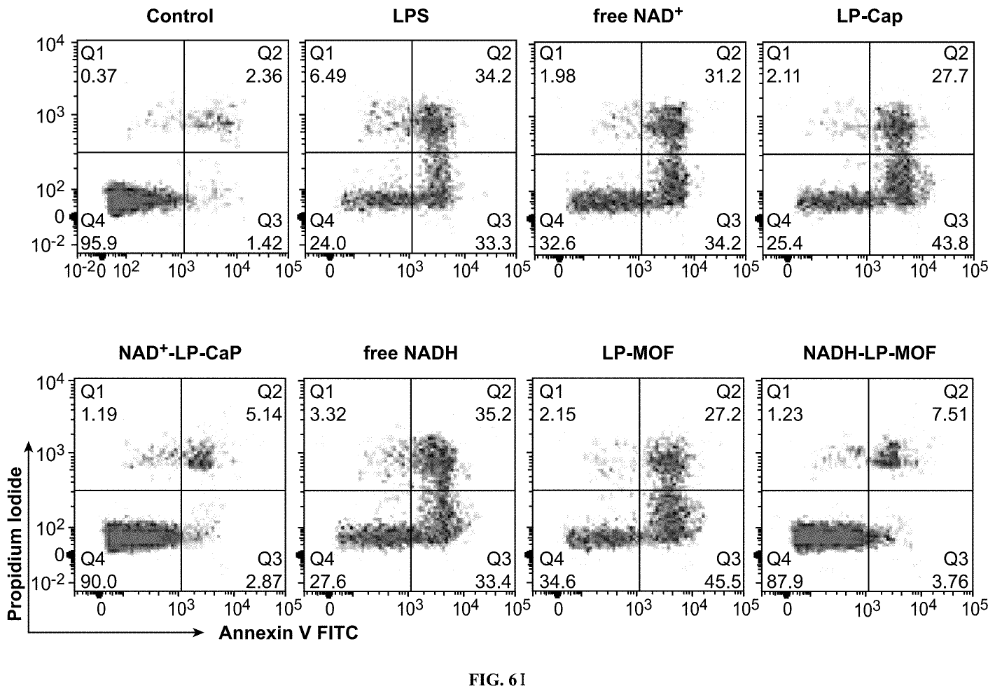

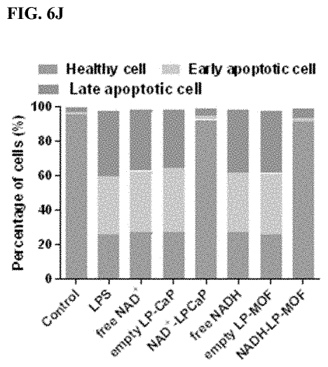

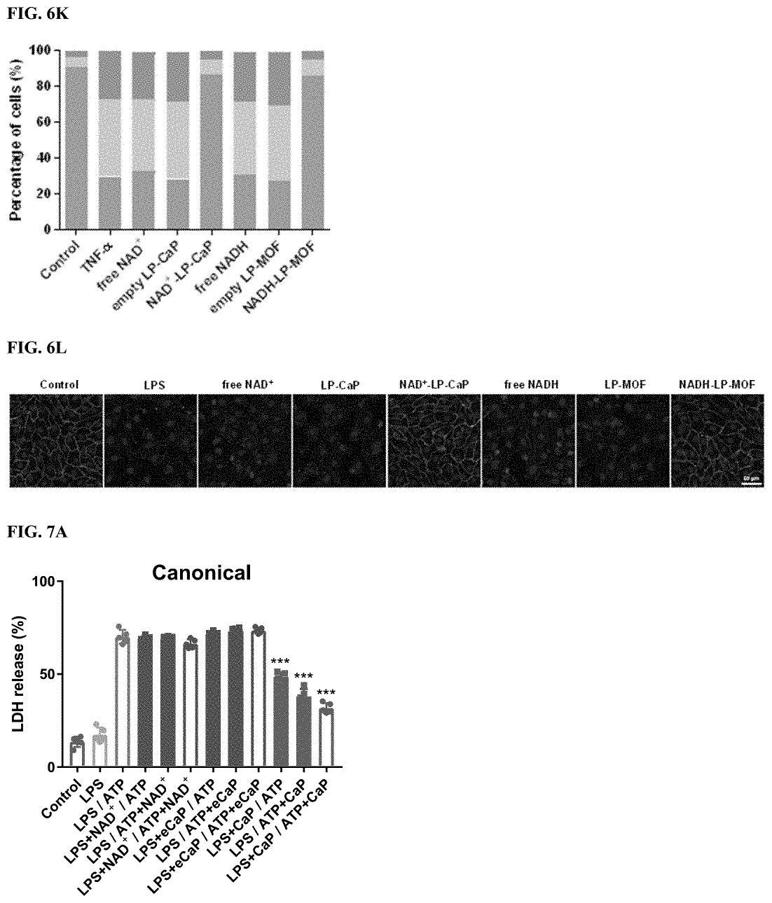

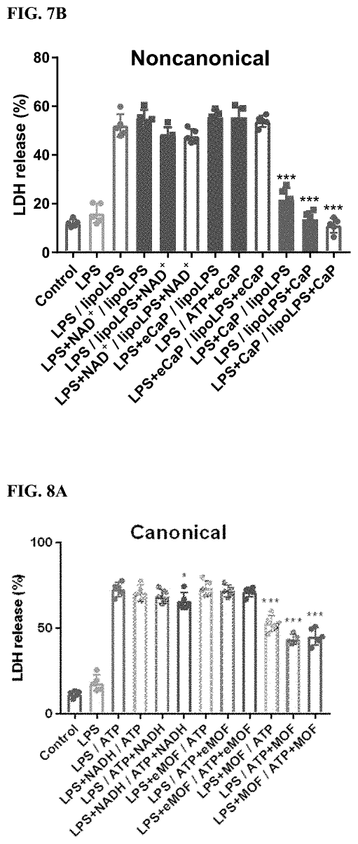

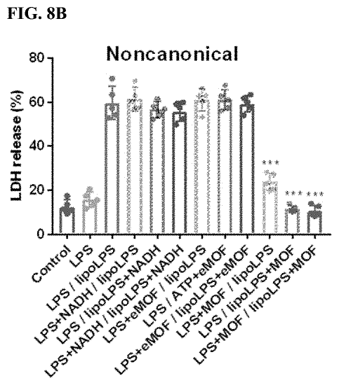

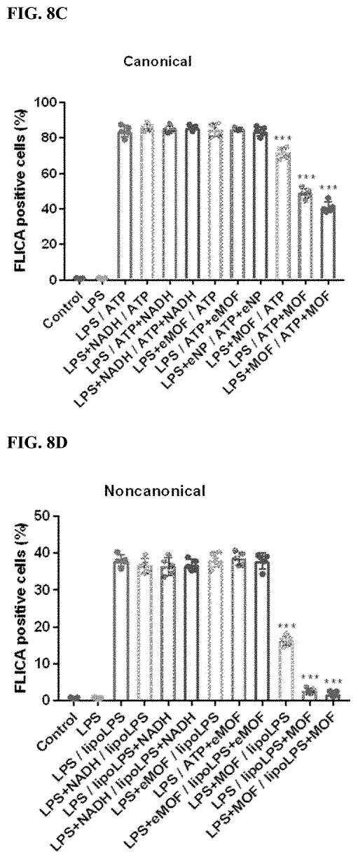

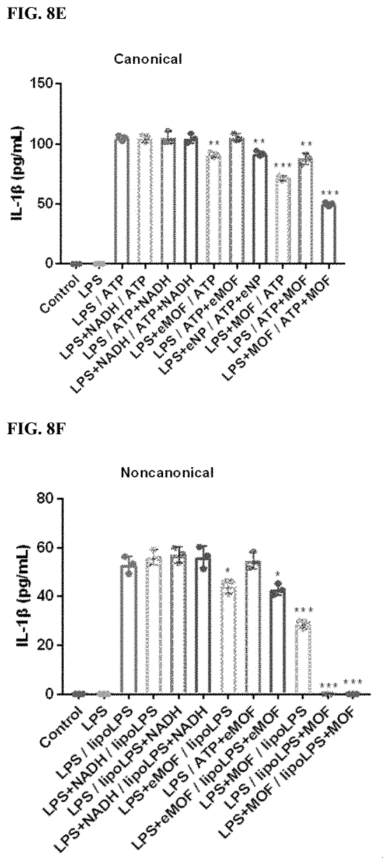

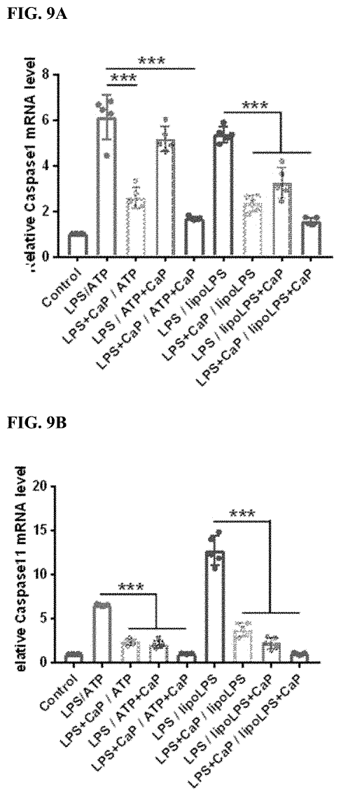

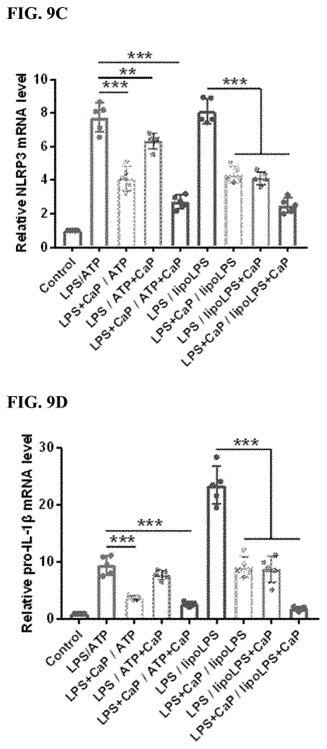

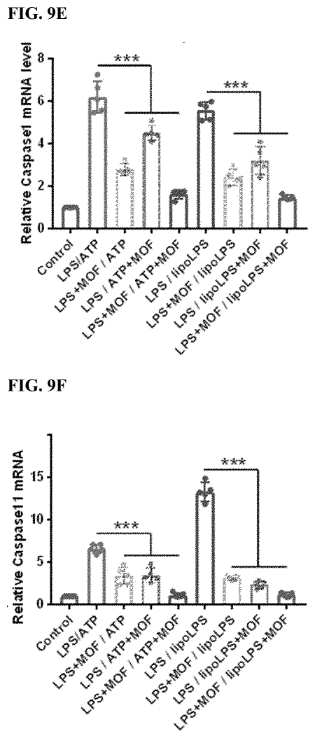

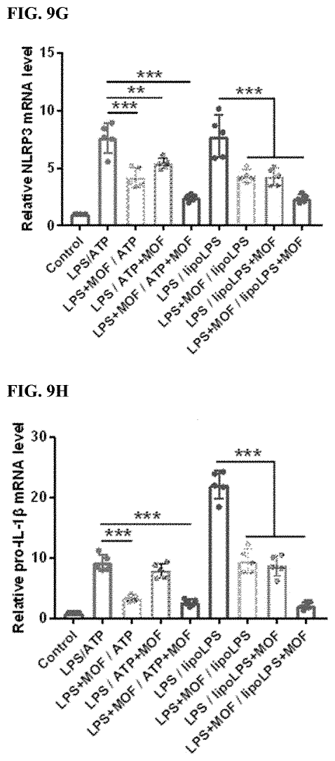

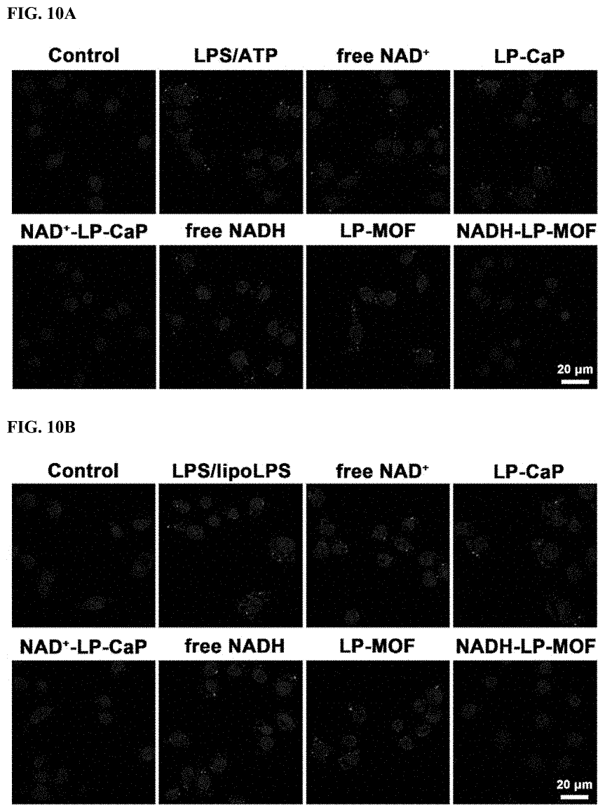

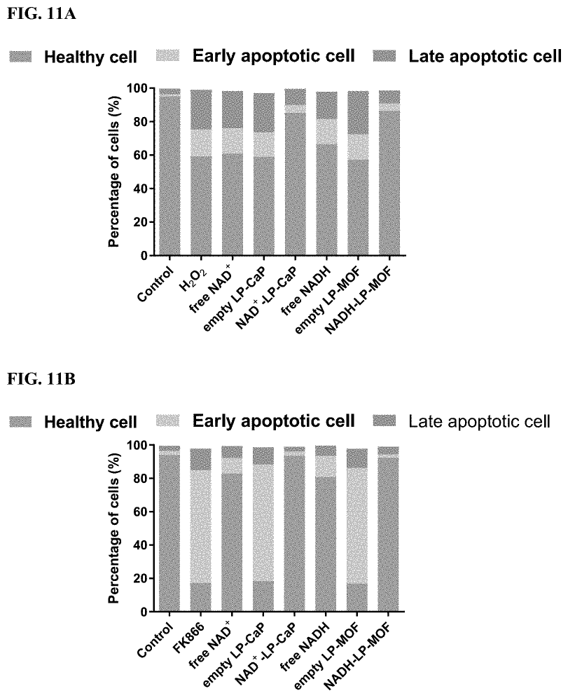

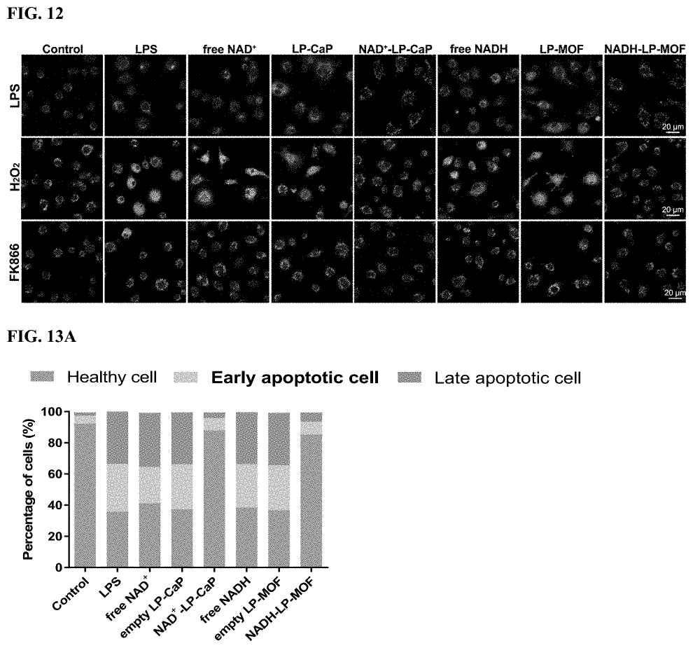

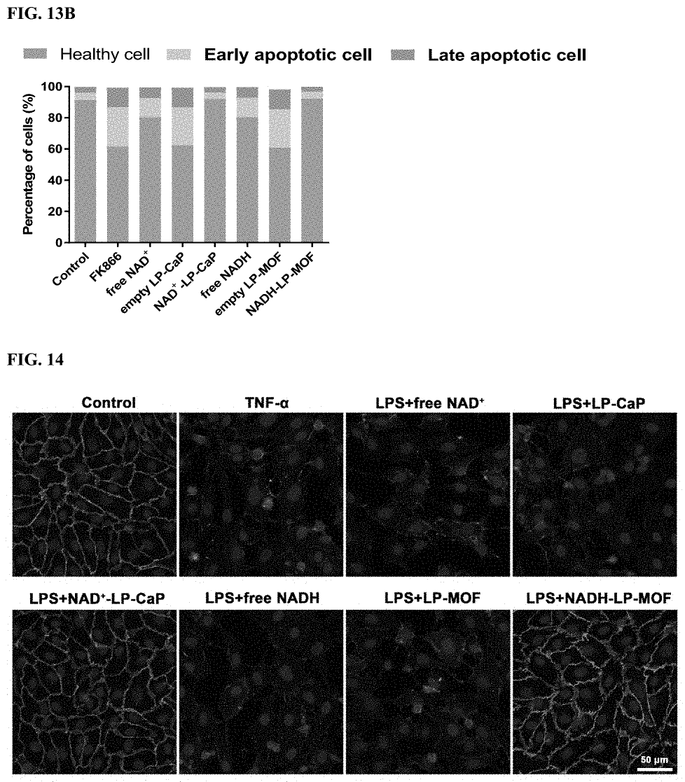

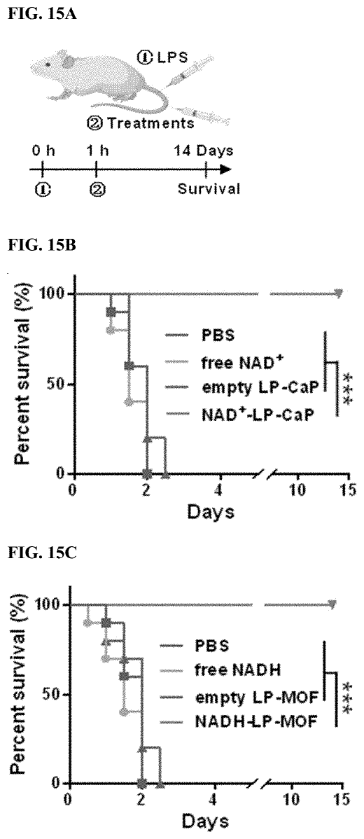

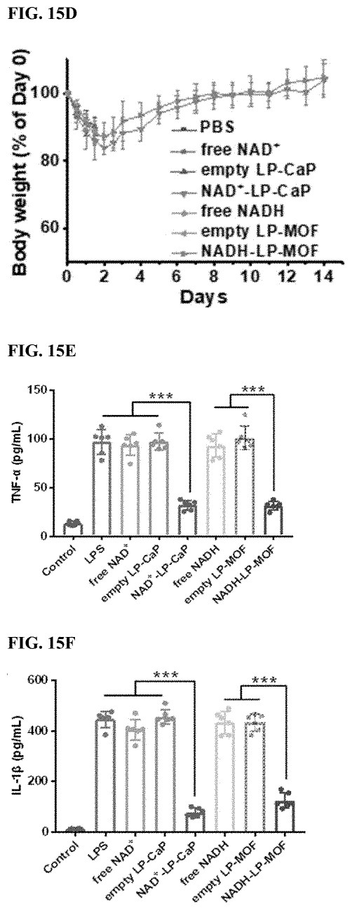

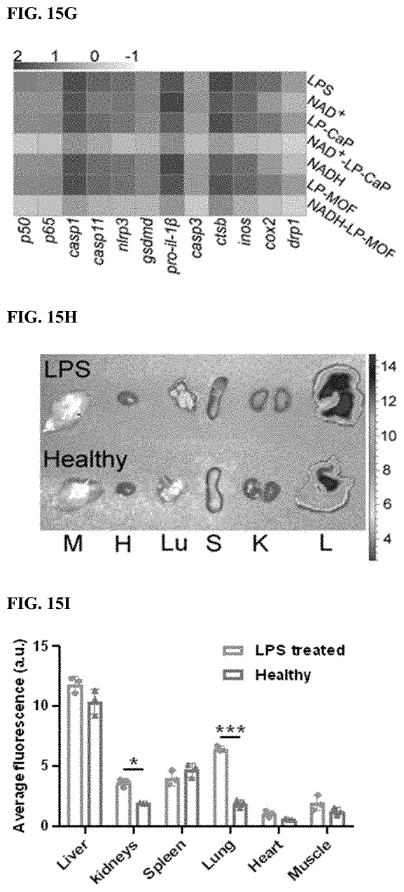

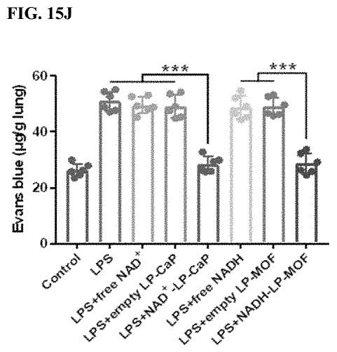

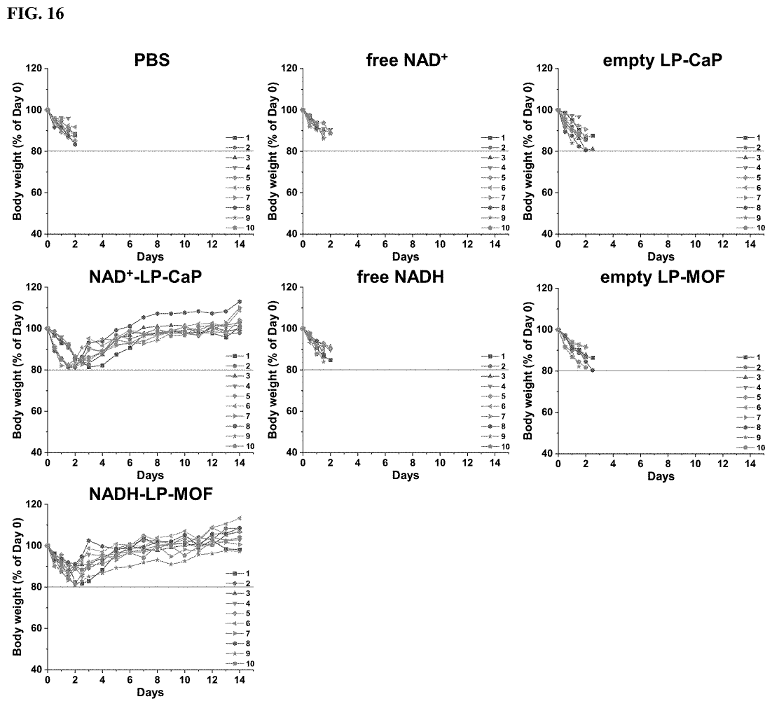

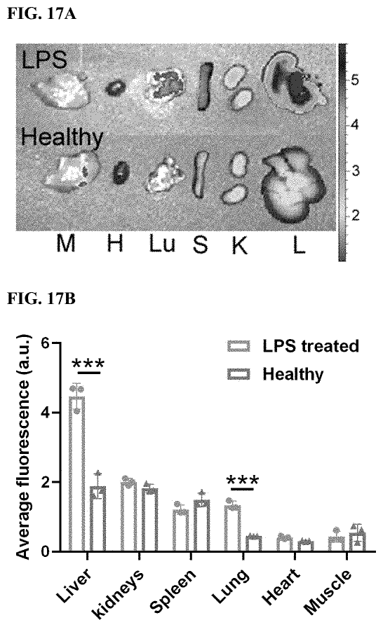

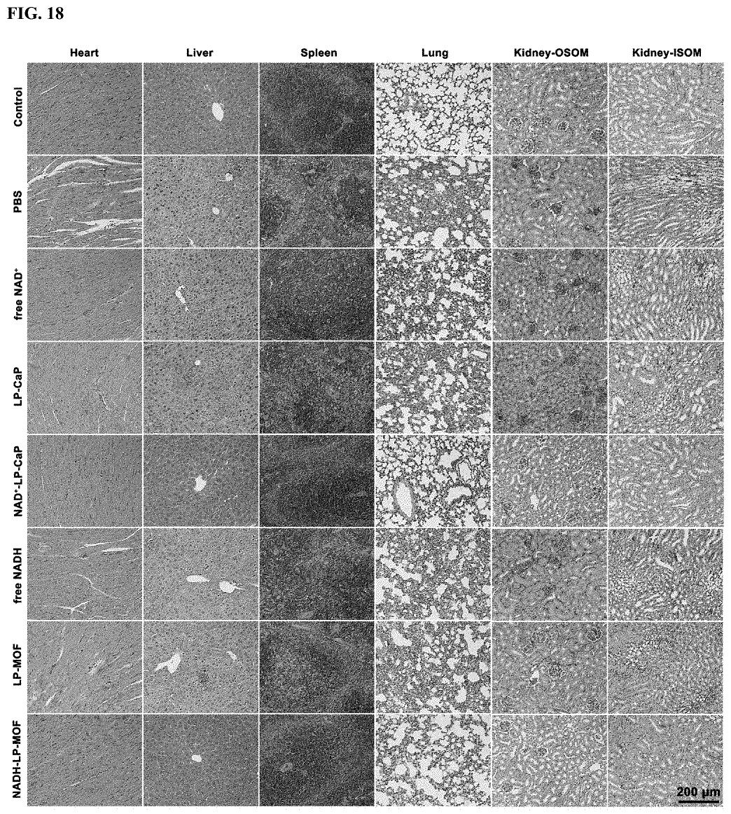

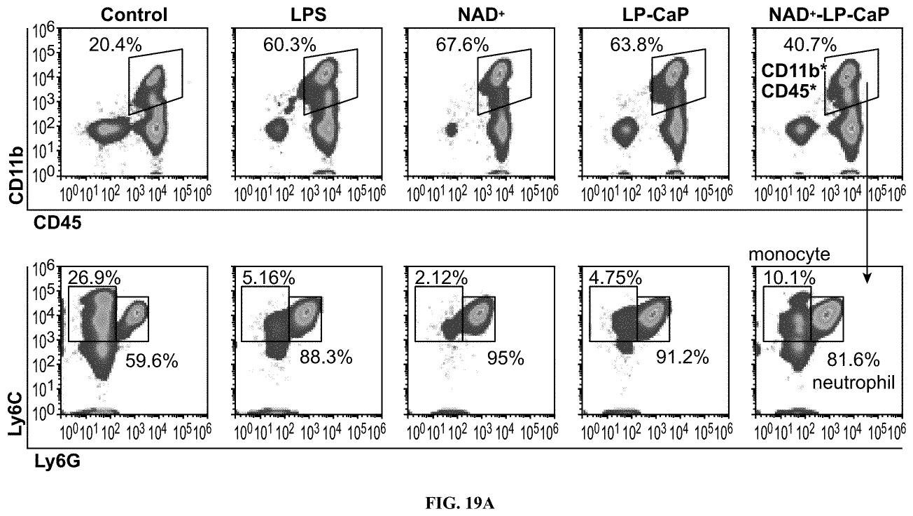

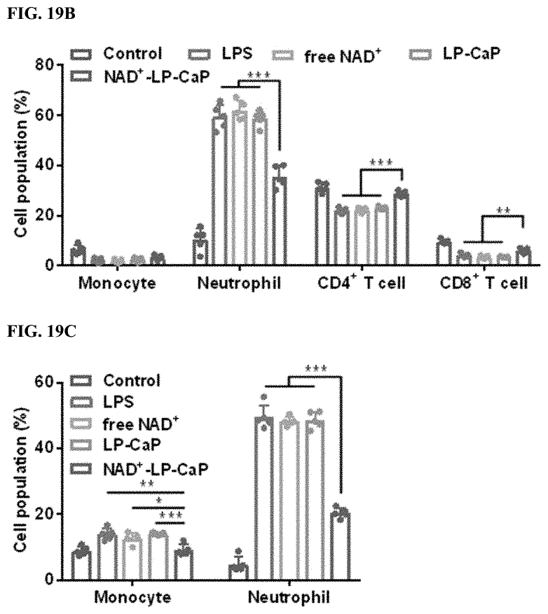

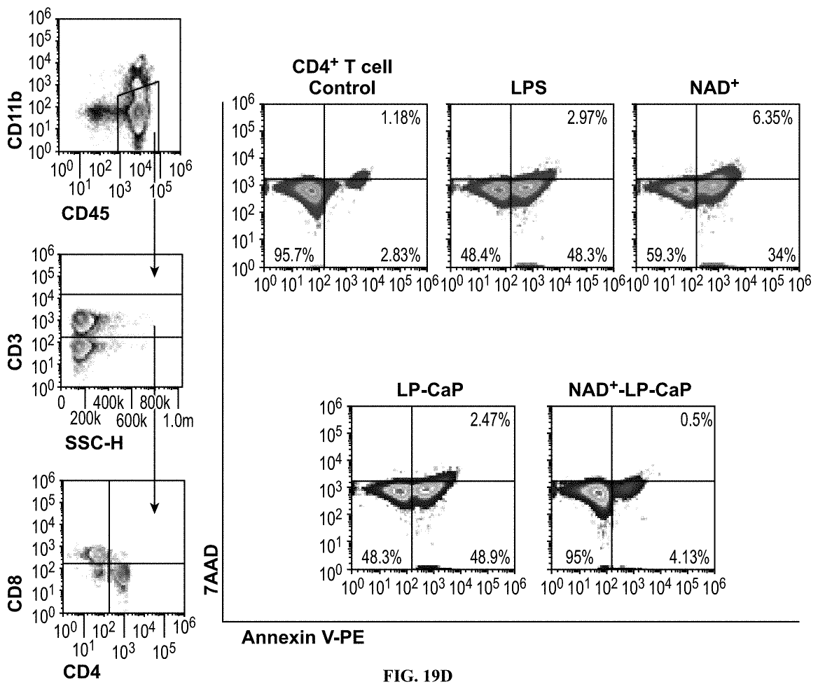

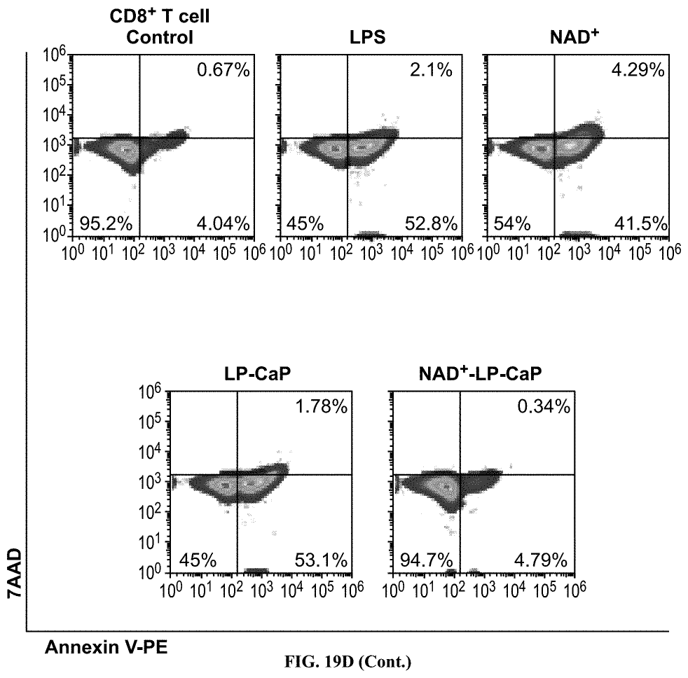

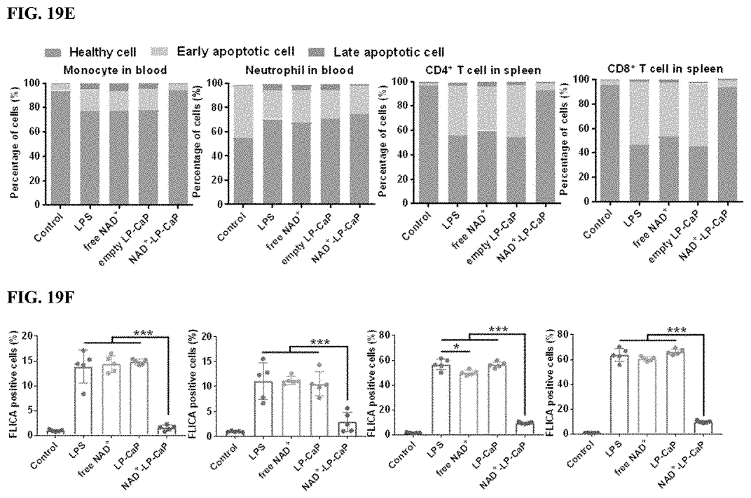

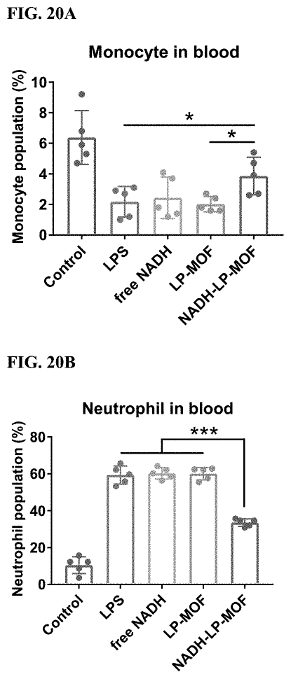

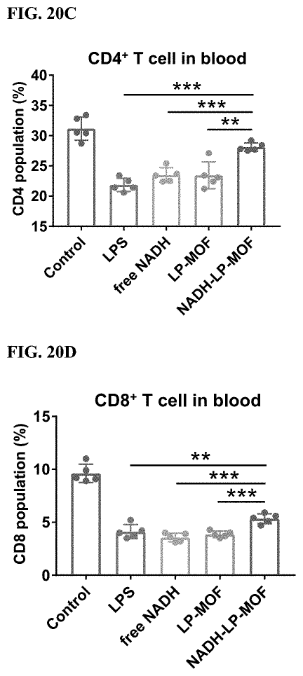

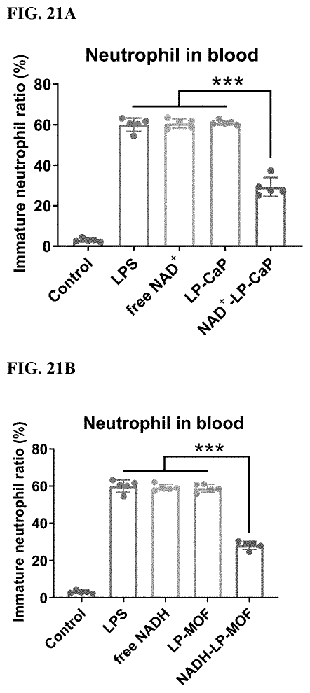

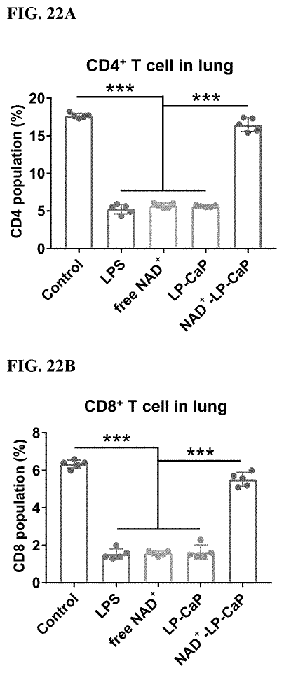

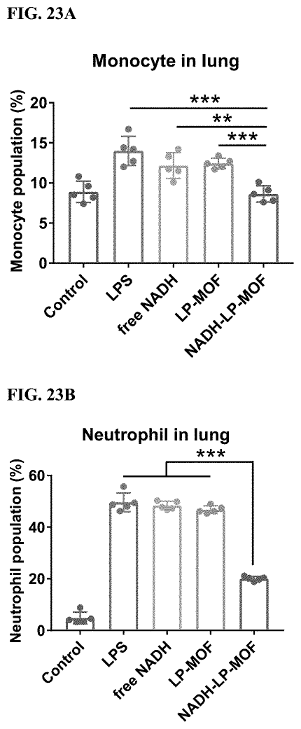

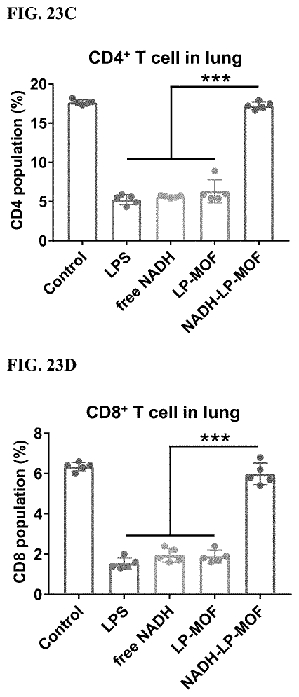

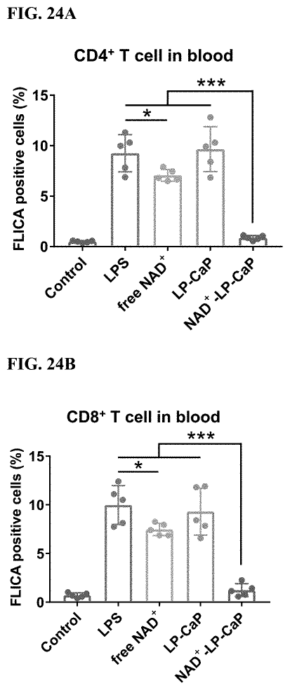

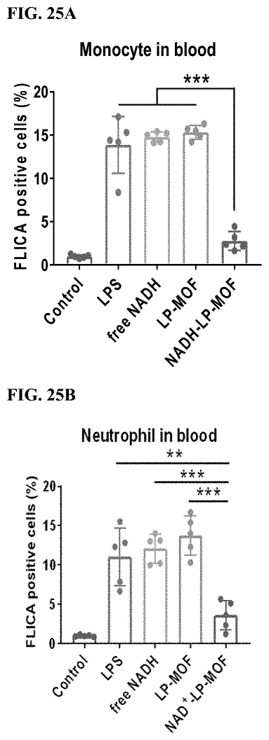

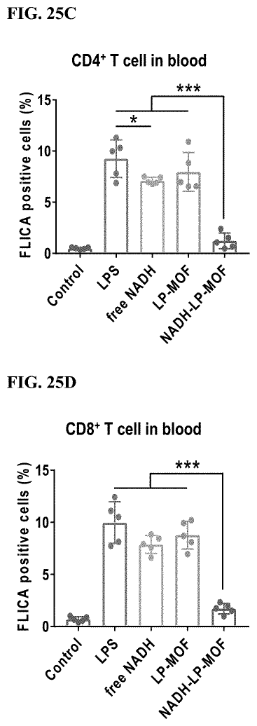

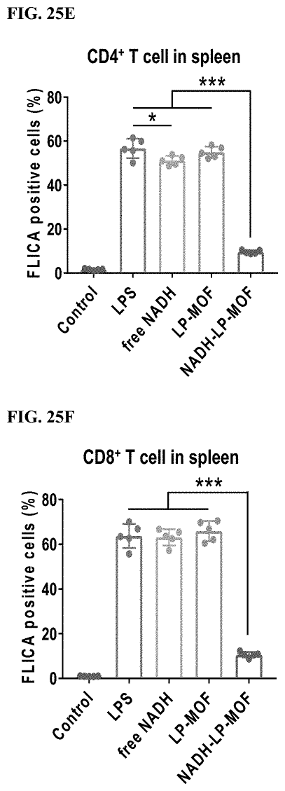

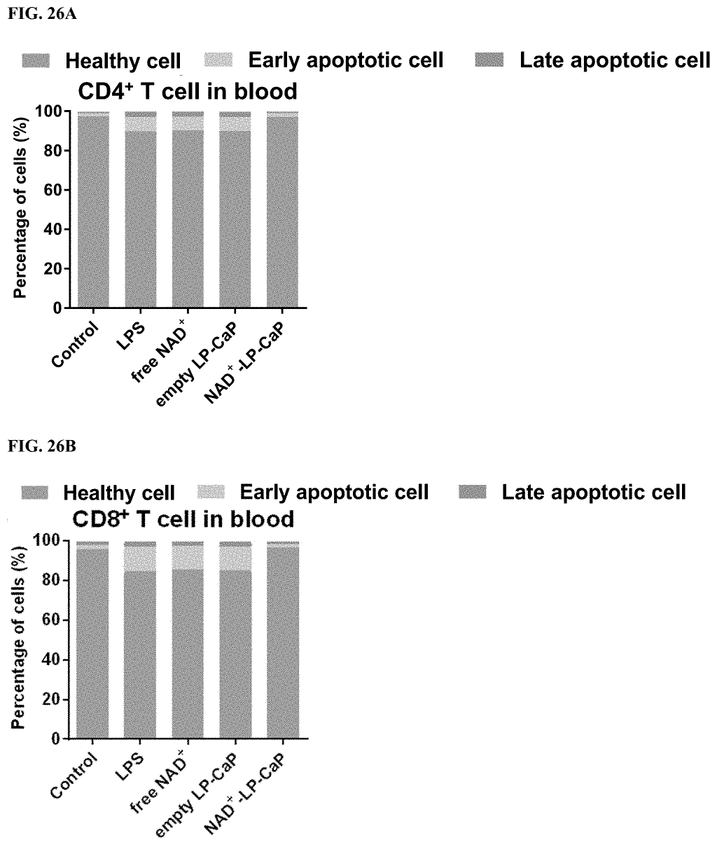

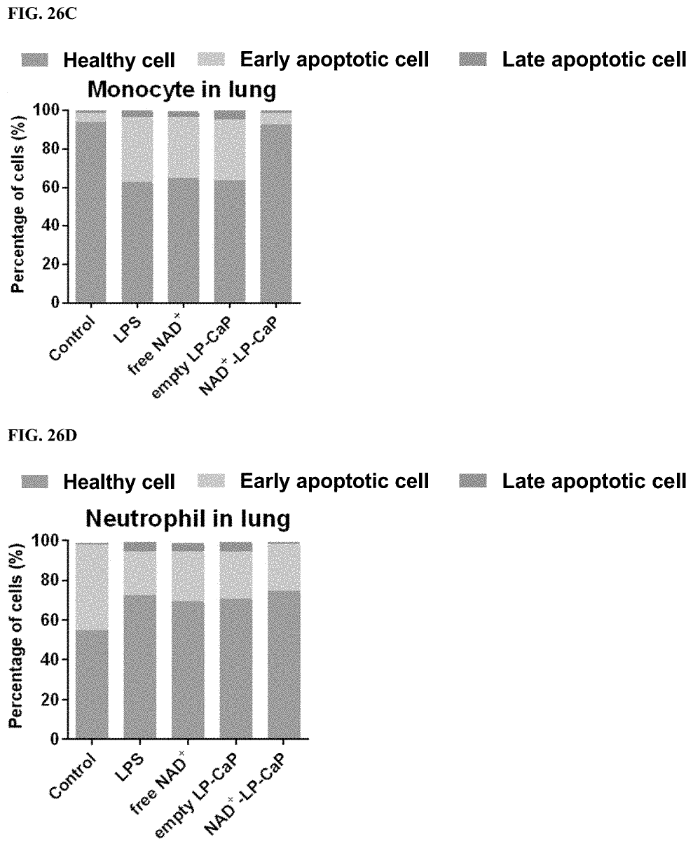

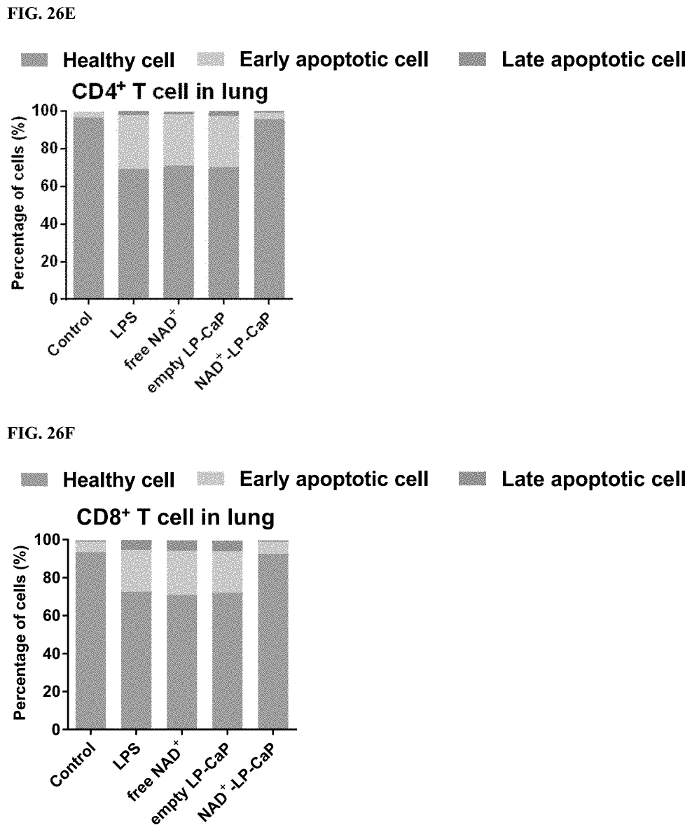

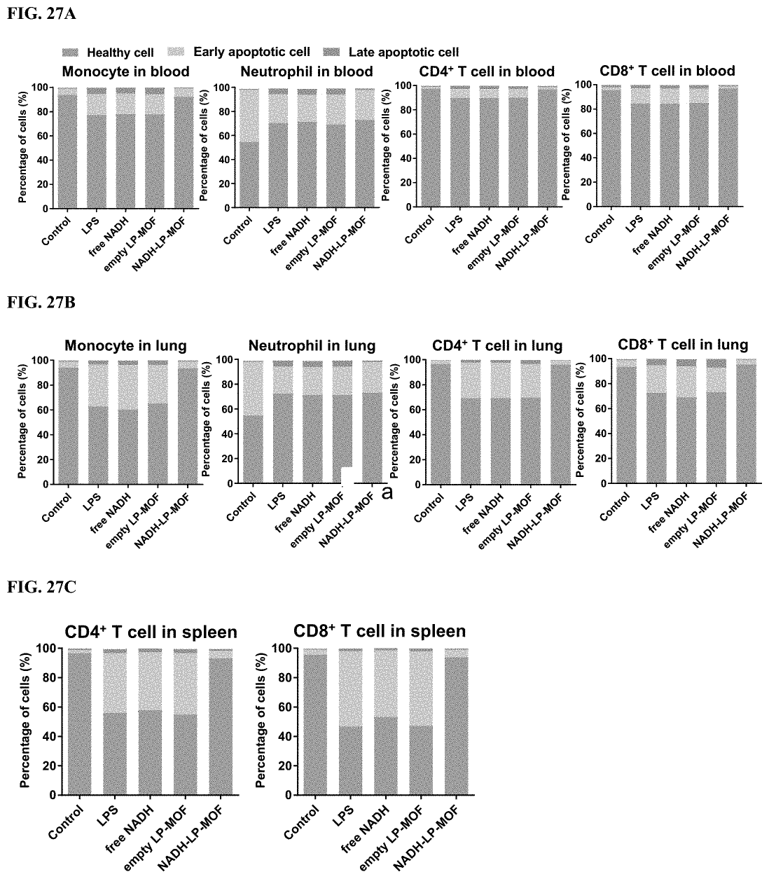

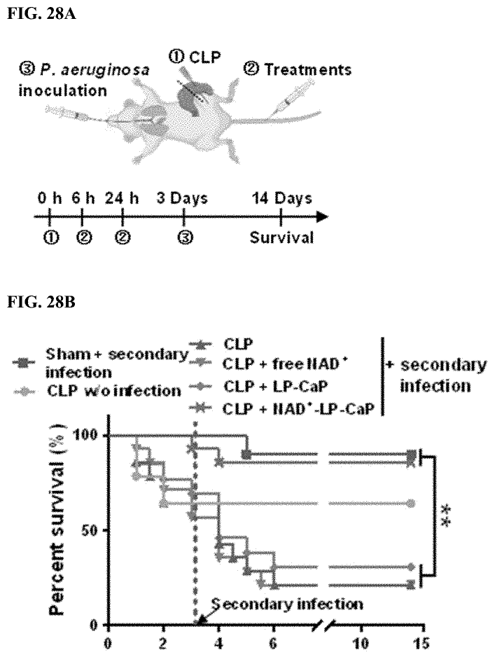

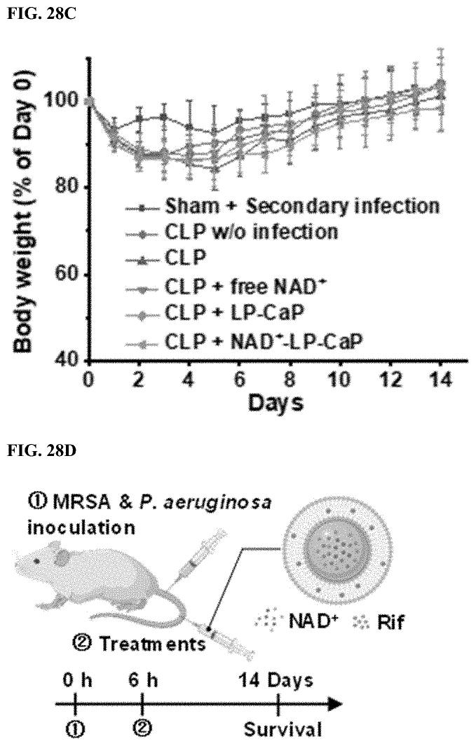

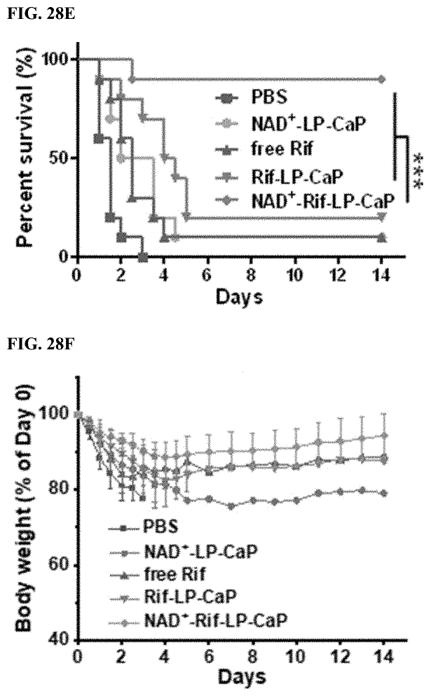

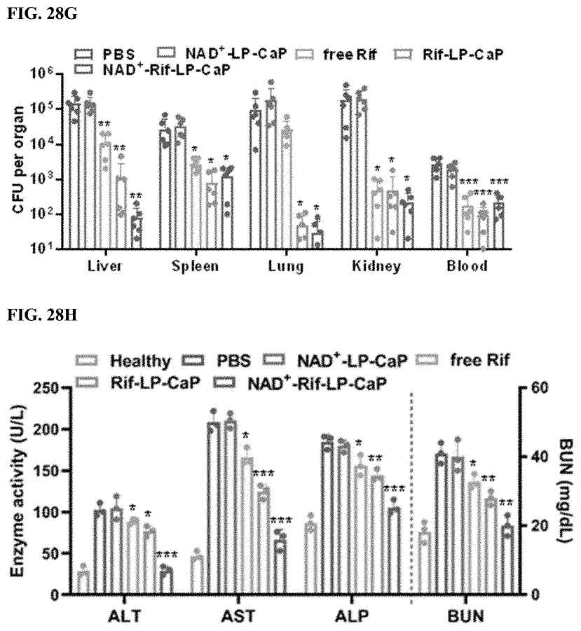

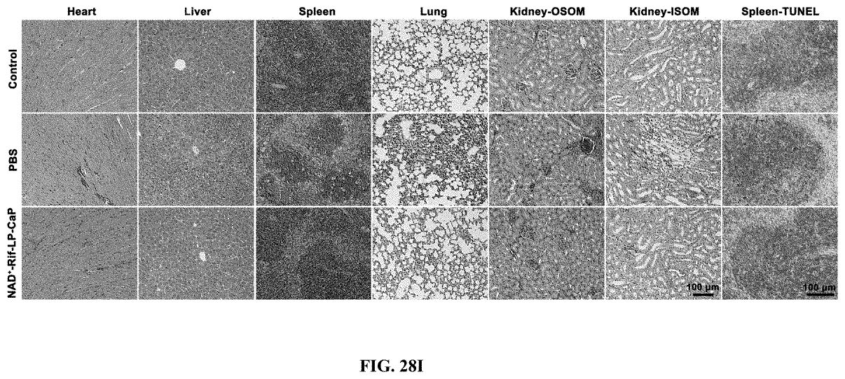

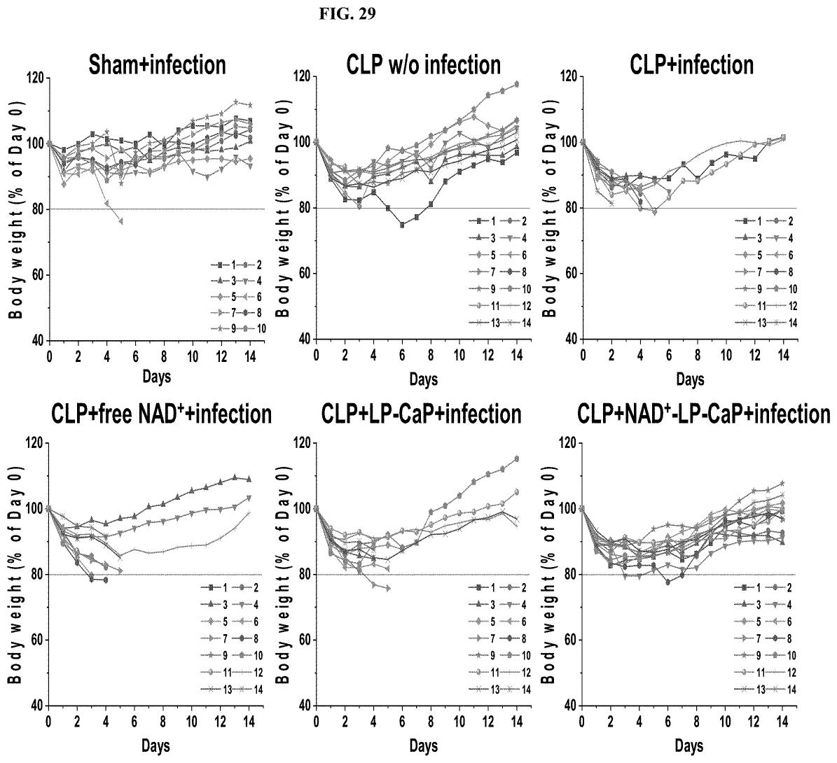

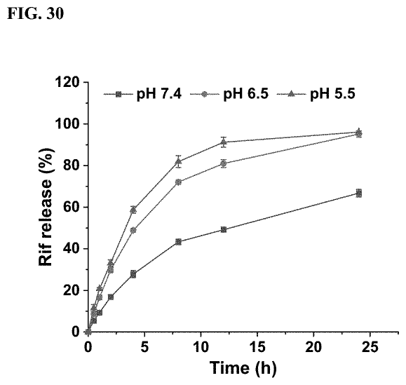

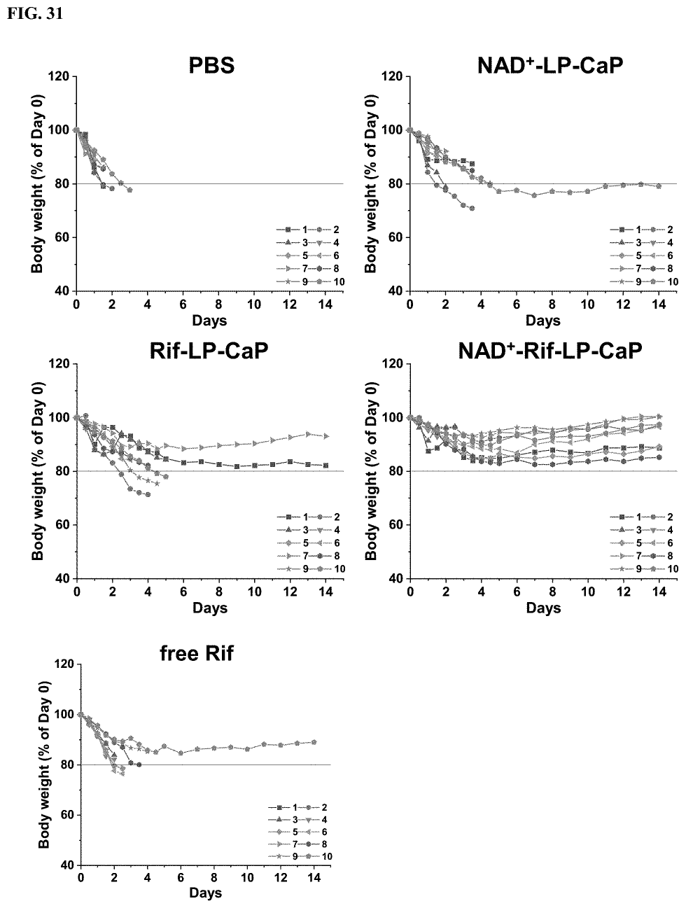

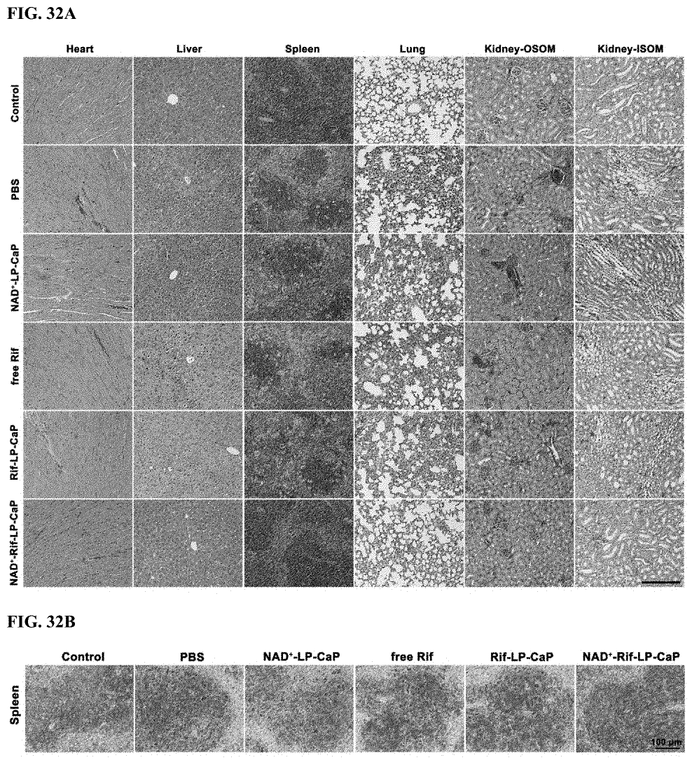

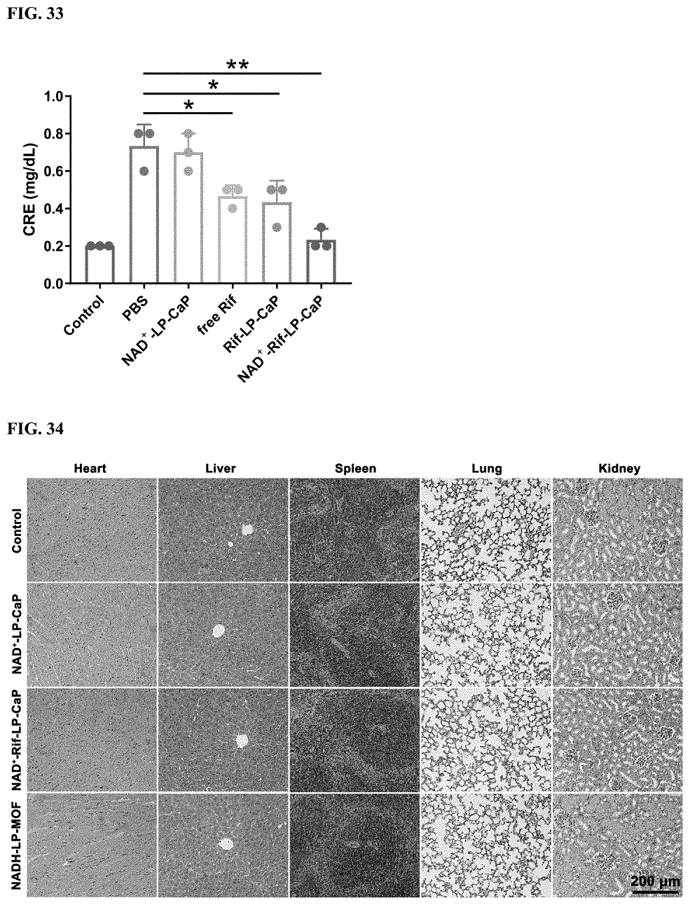

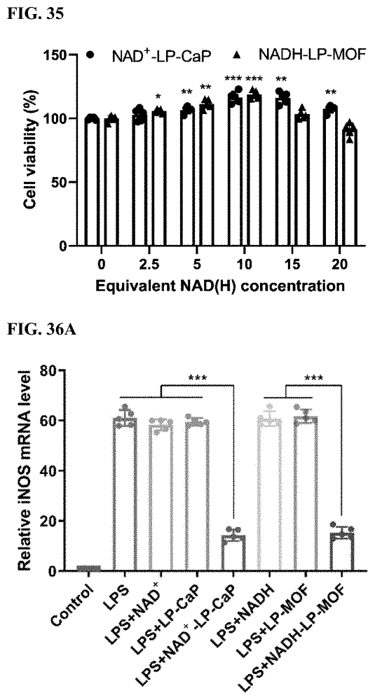

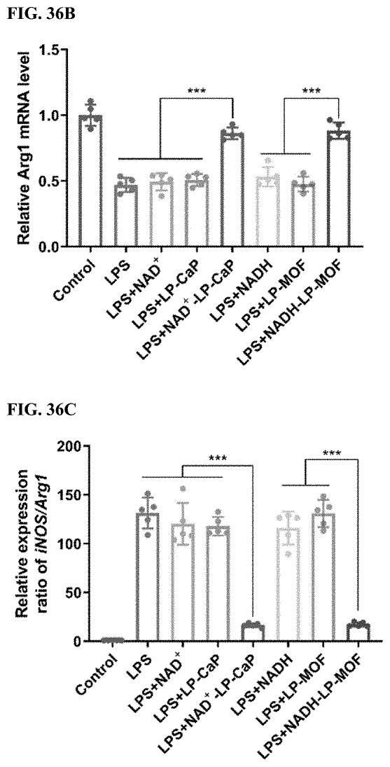

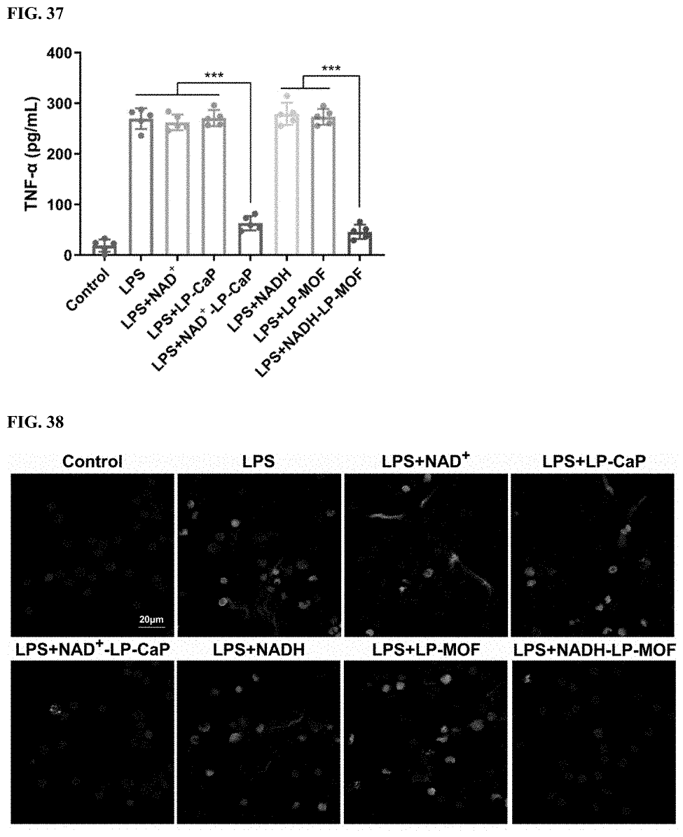

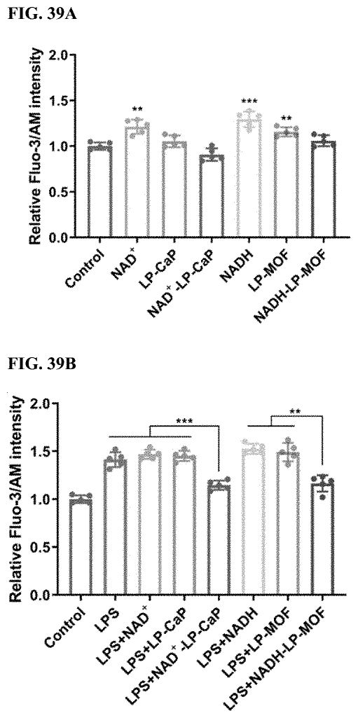

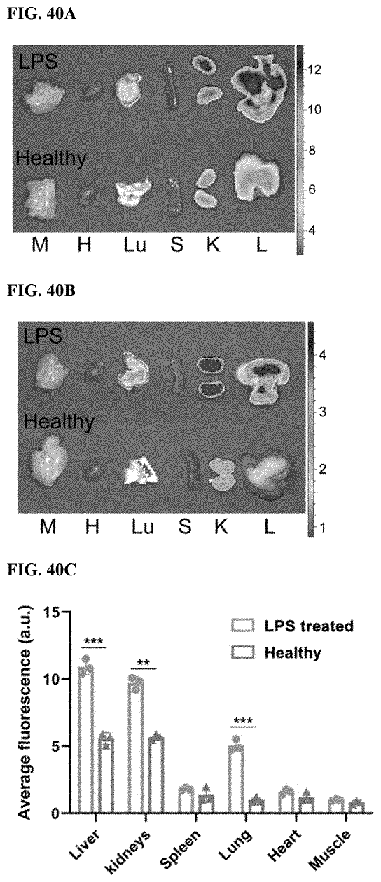

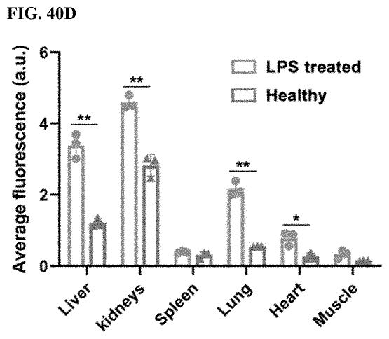

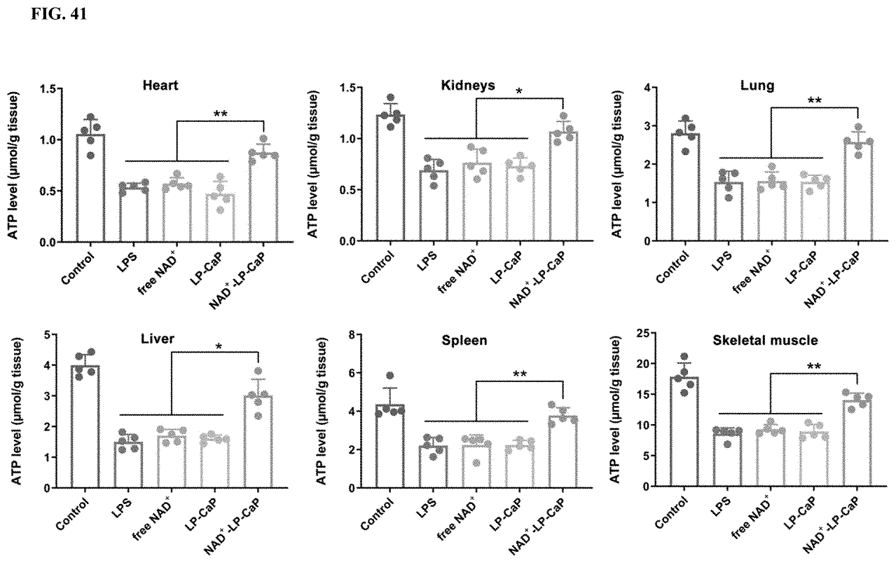

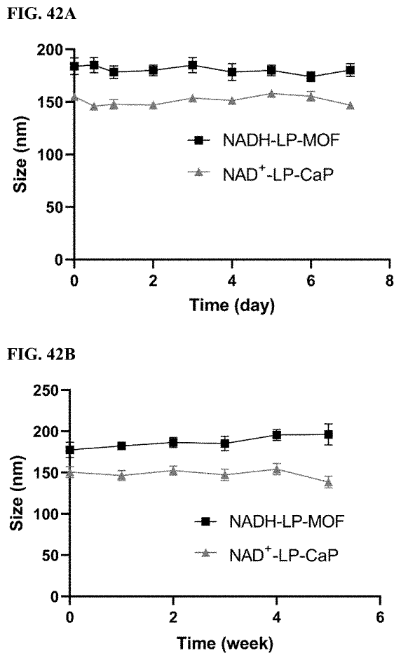

Although NAD⁺ is linked to inflammation, whether NAD⁺ suppresses or fuels inflammation was unclear prior to this work. To answer this question, LPS-stimulated BMDM was treated with the NAD(H)-loaded NPs, and both NAD⁺-LP-CaP and NADH-LP-MOF significantly reduced the production of pro-inflammatory cytokines TNF-α and IL-6 (FIGS. 6A, 6B), thus demonstrating a strong anti-inflammatory effect induced by cytosolic delivery of NAD⁺ or NADH. Empty NPs also slightly reduced cytokine production, consistent with the observation that lipid-coated nanoparticles can absorb LPS partially neutralizing its inflammatory effects. In contrast, free NAD⁺ and NADH did not affect the production of these pro-inflammatory cytokines, which can be attributed to the fact that free NAD(H) cannot be taken up by cells directly.

Inflammasomes play a central role in activating the inflammatory responses against infections. The impact of NAD(H)-loaded NPs on inflammasome activation was assessed. NLRP3 inflammasome activation is known to require two signals: a priming signal (signal 1) that stimulates the transcription of a set of inflammasome related proteins; and an activation signal (signal 2) provided by a variety of pathogen-associated molecular patterns (PAMPs) or damage-associated molecular patterns (DAMPs) that initiate inflammasome oligomerization. In this study, LPS was used for signal 1, while ATP and cytosolic delivered LPS by lipofectamine 2000 (lipoLPS) were used as signal 2 for “canonical” and “non-canonical” inflammasome pathways, respectively. BMDM were incubated with the NAD(H)-loaded NPs together with either signal 1 or signal 2 or both, cell pyroptosis (indicated by the level of lactate dehydrogenase (LDH) released), caspase1 activation (a key enzyme in the inflammasome pathways that cleaves and activates downstream targets such as gasdermin D (GSDMD) and pro-inflammatory cytokines, monitored by a fluorochrome-labeled inhibitors of caspases (FLICA) probe FAM-YVAD-FMK (SEQ ID NO: 44)), as well as the IL-1(3 production level were studied to investigate inflammasome activation. The NAD(H)-loaded NPs, when co-incubated with both signal 1 and signal 2, resulted in a significant reduction in cell pyroptosis (FIGS. 7-8), caspase1 activation (FIGS. 6C, 6D), and IL-1(3 production (FIGS. 6E, 6F), demonstrating their blockage to both priming signal and activation signals. It is worth noting that compared with around 50% reduction in IL-1(3 release level for the canonical pathway, the NPs completely blocked the IL-1(3 production and recovered the LDH release to the baseline level in the non-canonical pathway, demonstrating more efficient suppression of this pathway. In comparison, 10 μM free NAD(H) did not inhibit non-canonical inflammasome activation in BMDM when added together with the stimuli. The difference may be attributed to the inefficient NAD⁺ uptake by the cells.

To further investigate the effect of intracellular NAD⁺ delivery to inflammasome gene transcription, NF-κB p65 activation was observed by CLSM. As shown in FIGS. 6G and 6H, p65 nuclear translocation induced by LPS was decreased by both NAD⁺-LP-CaP and NADH-LP-MOF treatments, which is in line with previous research on NAD⁺ precursor, but with improved efficacy. Moreover, the transcription levels of multiple inflammasome-related genes (including casp1, casp11, nlrp3 and pro-IL-1β) were studied by qPCR (FIGS. 9A-9H) and they were all significantly reduced by the NAD(H)-loaded NPs, which further corroborate with the NF-κB activation study. ASC (Apoptosis-associated speck-like protein containing a caspase recruitment domain) oligomerization triggered by signal 2 indicates NLRP3 inflammasome assembling and activation. The NAD(H)-loaded NPs attenuated the ASC speck numbers in BMDMs induced by LPS/ATP, but they nearly blocked the ASC speck formation induced by LPS/lipoLPS, indicating a stronger inhibition to the non-canonical inflammasome pathway (FIGS. 10A, 10B). Sirtuins, a family of NAD⁺-dependent lysine-deacetylases, can negatively regulate NF-κB and NLRP3 activation. While not wishing to be bound by theory, since NAD⁺ is a principal regulator of sirtuin activity, it may be that the NAD(H)-loaded NPs suppress inflammation through sirtuin pathways.

Macrophages challenged by pathogens are often polarized towards proinflammatory M1 phenotype helping aid in defense against the pathogen, but also potentially leading to organ injury. Thus, it is important to modulate the M1/M2 polarity during bacterial infection and sepsis. To investigate whether the NAD(H)-loaded NPs can affect macrophage polarization, BMDM mRNA expression of inducible nitric oxide synthase (iNOS) and arginase 1 (Arg1) were monitored as their ratio (iNOS/Arg1) is known as an M1/M2 indicator. While LPS stimulation promoted M1 polarization, the NAD(H)-loaded NPs significantly reduced the iNOS/Arg1 ratio, indicating its strong anti-inflammatory effect (FIGS. 36A-36C). In addition to macrophages, we also studied the NPs' effect on neutrophils as they play an important role in systemic inflammation. Similar to what was observed with the macrophages, the NAD(H)-loaded NPs significantly reduced neutrophil TNF-α production (FIG. 37) and blocked NETosis (FIG. 38), a type of programmed cell death resulting in the formation of inflammatory neutrophil extracellular traps (NETs). Since excessive release of NETs can be detrimental to the hosts, resulting in inflammation, thrombosis, and multiple organ failure, the NAD(H)-loaded NPs blocked formation of NETs and thus could ultimately contribute to decreased vascular dysfunction and multiorgan injury induced by sepsis. In contrast to the NAD(H)-loaded NPs, free NAD(H) and the empty NPs did not show any significant anti-inflammatory effect on macrophages and neutrophils.

Inflammation-induced cell apoptosis is a key factor causing multiple organ damage and immune suppression during sepsis. The effect of the NAD(H)-loaded NPs on cell apoptosis was also investigated. As shown in FIGS. 6I, 6J and FIG. 1I, the NAD(H)-loaded NPs drastically inhibited cell damage and apoptosis induced by multiple factors such as inflammation (LPS), oxidative stress (H₂O₂), or NAD⁺ depletion (FK866). The effect of the NAD(H)-loaded NPs on calcium flux was investigated, as calcium is an important second messenger regulating inflammation and cell death (FIGS. 39A-39B). LPS can induce endoplasmic reticulum stress and disrupt cellular calcium homeostasis, which is associated with mitochondrial damage and cell apoptosis. The NAD(H)-loaded NPs significantly reduced cytosolic calcium concentration in LPS-stimulated BMDMs, consistent with the findings from the NF-κB activation and apoptosis studies.

The effect of the NAD(H)-loaded NPs on BMDM mitochondrial membrane potential (ΔΨm) was also investigated using a JC-1 fluorescence probe. The NAD(H)-loaded NPs effectively prevented ΔΨm loss (FIG. 12). Since inflammation-induced NAD⁺ depletion will lead to mitochondrial dysfunction, a key event that initiates apoptosis, improving NAD⁺ supply through the NAD(H)-loaded NPs can reverse this process and thus protecting cells from death. In addition to the immune cells, the NAD(H)-loaded NPs also prevented TNF-α triggered HUVECs apoptosis (FIG. 6K), suggesting that their therapeutic benefit can be universally applied to many types of cells against different pro-inflammatory factors (FIGS. 13A, 13B).

Sepsis-induced endothelial damage and the resultant hypotension is a major cause of mortality. Bacterial toxin and pro-inflammatory cytokines can destabilize endothelial cell-cell interactions, thus resulting in vascular leakage and subsequent shock as well as organ failure. The ability of NAD(H)-loaded NPs to maintain vascular homeostasis and prevent endothelial damage was investigated. Vascular endothelial cadherin (VE-cadherin) is the key protein forming adherens junctions and maintaining vascular integrity. Both LPS and TNF-α disrupted VE-cadherin and destroyed the confluent endothelial monolayer. Incomplete endothelium loses its barrier function and induces vascular leakage and shock. The NAD(H)-loaded NPs effectively prevented VE-cadherin dissociation and endothelial barrier disruption induced by both bacterial cell wall component and proinflammatory cytokine (FIG. 6L; FIG. 14), suggesting their remarkable therapeutic benefit to the vascular system. In contrast, free NAD(H) and the empty NPs did not show much benefit.

### Example 5—Therapeutic Efficacy in an Endotoxemia (LPS-Induced In Vivo Sepsis) Model

The in vivo therapeutic efficacy of the NAD(H)-loaded NPs was tested in a well-established LPS-induced endotoxemia sepsis model (FIGS. 15A-15D). Mice that received LPS via intravenous (IV) administration (15 mg/kg) all died within 3 days. Free NAD(H) and NPs without NAD(H) showed no increase in survival rate. However, a single injection of either NAD⁺-LP-CaP or NADH-LP-MOF 1 h after LPS administration was able to rescue 100% of the septic mice (n=10). Although notable body weight loss was initially observed, the mice treated with NAD(H)-loaded NPs all started to recover after day 3 (FIG. 16).

To further evaluate the therapeutic efficacy of the NAD(H)-loaded NPs, the cytokine levels in the plasma of LPS-induced septic mice were tested by ELISA (FIGS. 15E, 15F). The concentration of pro-inflammatory cytokines TNF-α and IL-1(3, the key factors responsible for cytokine storm and high mortality in sepsis patients, were drastically reduced after NAD(H)-loaded NP administration. Meanwhile, multiple pro-inflammatory gene expressions in white blood cells (WBCs) were also decreased by the NAD(H)-loaded NPs (FIG. 15G), suggesting an improved immune status in these mice. In addition to the inflammasome related genes such as p65, nlrp3, and gsdmd, the expression of a number of other genes including caspase3 (casp3), dynamin-related protein 1 (drp1), cathepsin b (ctsb), and cyclooxygenase-2 (cox-2) were also down-regulated by the NAD(H)-loaded NPs, demonstrating their broad influence on a variety of pathways related to cell apoptosis, mitochondrial dysfunction, lysosomal biogenesis, and prostanoids release during sepsis.

The in vivo distribution of the LP-CaP NP and LP-MOF NP was investigated in both healthy mice and LPS-induced septic mice. Mice were IV injected with Cy5.5-labeled LP-CaP or LP-MOF NPs and sacrificed 4 h or 24 h after the administration, and tissues were collected for ex vivo imaging (FIGS. 15H, 15I; FIGS. 17A-17B; FIGS. 40A-40B). For both NPs, the liver accumulated most of the NPs in healthy and in the LPS-treated mice. However, the liver in septic mice cleared the NP much more slowly than that in the healthy ones, which was possibly attributed to the compromised liver function. In addition to the liver, the septic mice had significantly increased NP distribution in their lungs and kidneys likely due to endothelial damage and the resultant hyperpermeability. Since liver, lungs and kidneys more vulnerable to sepsis, leading to severe complications with high morbidity and mortality, the preferred NP accumulation in these organs allowed the NAD(H)-loaded NPs to function more effectively, thereby minimizing the damage (FIG. 18). Additionally, the ATP levels in all major organs of LPS-treated mice were measured, as energy dysfunction is associated with sepsis-mediated organ failure. While inflammation induced by LPS impaired the energy supply in all major organs, NAD⁺-LP-CaP effectively recovered the ATP levels (FIG. 41), again demonstrating a strong protective effect.

To investigate the NP's efficacy in ameliorating lung injury and vascular leakage, pulmonary vascular permeability was evaluated by Evans blue dye-labeled albumin (FIG. 15J). The extravasation of Evans blue dye out of the pulmonary vasculature was significantly increased after LPS administration. Compared to administering free NAD(H) or empty NPs that did not show any efficacy, a single injection of either NAD⁺ or NADH-loaded NPs reduced Evans blue leakage to the baseline, suggesting their great efficacy in maintaining endothelial function and integrity.

The immune response in sepsis can be characterized by a cytokine-mediated hyper-inflammatory phase, and a subsequent immune-suppressive phase⁴⁵. During the hyperreactive immune response phase, the innate immune system (e.g., neutrophils and monocytes) is activated to kill invading pathogens, but hyperactive immune response can also lead to multiple organ injuries. Sepsis-induced immunosuppression has been attributed to a number of mechanisms including immune cell apoptosis and dysfunction and it makes patients susceptible to nosocomial infections. To evaluate the impact of the NAD(H)-loaded NPs on the mouse immune system, immune cell population and metabolic status in LPS-induced septic mice were monitored by flow cytometry. As shown in FIG. 19A, FIG. 19B and FIG. 20, NAD(H)-loaded NPs moderated the neutrophil surge in blood induced by LPS, suggesting a strong anti-inflammatory effect. The immature/total neutrophil ratio is an indicator of systemic inflammation and the severity for sepsis⁴⁷. While very few neutrophils in healthy mice (3%) were immature neutrophils the immature/total neutrophil ratio in septic mice increased to 60%, and this number decreased to 29% after the treatment of NAD(H)-loaded NPs (FIGS. 21A, 21B). In addition to the blood, NAD(H)-loaded NPs significantly reduced the infiltration of macrophage and neutrophil in the pulmonary alveoli (FIG. 19C; FIGS. 22-23). This finding is in line with the reduced vascular permeability determined by the Evans blue assay, and serves as an indication for reduced organ and tissue damage induced by the hyperactive immune response⁴⁸. Immune cell caspase1 activation evaluated by the FLICA assay was employed to further investigate the inflammatory status. Caspase1 is a pivotal trigger for cell pyroptosis, a highly inflammatory form of programmed cell death releasing pro-inflammatory cytokines and DAMPs to amplify the inflammatory response⁴⁹. The administration of NAD(H)-loaded NPs significantly reduced caspase1 activation in all types of immune cells in blood and spleen, while free NAD(H) or the empty NPs did not show an observable effect (FIG. 19F; FIGS. 24-25). This finding is consistent with the in vitro cell pyroptosis data (FIGS. 6C-6F; FIGS. 7-8) and in vivo gene expression data obtained by qRT-PCR (FIG. 15G). The reduced neutrophil population and caspase1 activation induced by the NAD(H)-loaded NPs demonstrated their high in vivo anti-inflammatory efficacy.

In contrast to pyroptosis, apoptosis is a type of programmed cell death immunologically “silent” or even anti-inflammatory. Immune cell apoptosis, especially the loss of lymphocytes, is a primary cause of sepsis-induced immunosuppression. The NAD(H)-loaded NPs effectively blocked monocyte and lymphocyte apoptosis in blood, lung, and spleen of the septic mice, while other treatments did not show observable improvement (FIGS. 19D, 19E; FIGS. 26-27). Notably, the lymphocyte apoptosis in spleen—which was more severe than that in blood—was drastically reduced from 50% to the base line by the NAD(H)-loaded NPs (FIG. 19D). The strong anti-apoptotic effect of the NAD(H)-loaded NPs effectively prevented lymphocyte loss during sepsis (FIG. 19B; FIGS. 22-23), which is of great significance in maintaining immune tension against primary and secondary infections. In contrast to monocytes or lymphocytes, neutrophil apoptosis was much higher under physiological conditions, but it was delayed during sepsis to achieve more efficient killing of invading pathogens. The NAD(H)-loaded NPs did not affect early apoptosis of neutrophils, but they reduced late apoptosis/pyroptosis (FIG. 19E; FIGS. 26-27). The efficient blockage of both pyroptosis and apoptosis proved that the NAD(H)-loaded NPs were able to prevent both hyperinflammation and immunosuppression, thus making them effective immunomodulators to treat sepsis. Since NAD⁺-LP-CaP and NADH-LP-MOF revealed similar effects for both in vitro and in vivo studies, the NAD⁺-LP-CaP was chosen as a representative system for the following studies.

### Example 6—Therapeutic Efficacy in Preventing Sepsis-Induced Persistent Injury and Immunosuppression Using a CLP Induced Sepsis Model with P. aeruginosa Secondary Infection

Sepsis patients, surviving the initial cytokine mediated hyper-inflammatory phase, may be prone to infections due to immune suppression and multiorgan injury. In fact, one-third patients who recovered from sepsis die during the following year because of complications. Thus, preventing inflammation induced complications (e.g., immunosupprestion) and nosocomial infections are of great significance during sepsis therapy. The immunomodulation effect of NAD⁺ replenishment and its ability to reduce persistent organ injury were further investigated on a cecal ligation and puncture (CLP)-induced sepsis model with P. aeruginosa secondary infection (FIG. 28A). The CLP infection model mimics human polymicrobial peritoneal infection. CLP infection initiated a hyper-inflammatory phase with roughly 30% mortality at its early stage, which gradually turned to an immunosuppression phase due to impaired immune system, thus making the animals less resistant to pathogen invasion. Two doses of free NAD⁺, empty LP-CaP, or NAD⁺-LP-CaP were IV administrated 6 h and 24 h after the surgery to modulate the immune function. Three days after CLP, P. aeruginosa was intratracheally inoculated to the mice to mimic the nosocomial infections. While the P. aeruginosa secondary infection was minimally lethal to healthy mice (with 10% mortality in the sham group), it caused a much higher mortality for in CLP treated mice likely due to a compromised immune system and organ injury (FIGS. 28B, 28C; FIG. 29). NAD⁺-LP-CaP not only mitigated the cytokine storm in the hyper-inflammatory stage, leading to a reduction in mortality (from 30% to 7% at day 3), but also prevented CLP-induced immunosuppression as evidenced by significantly reduced mortality (14%, n=14) from P. aeruginosa secondary infections compared with the untreated group (79%) at day 14. In contrast, free NAD⁺ and the empty LP-CaP did not improve the survival rate.

### Example 7—Therapeutic Efficacy in a Polymicrobial Blood Infection Model

Killing pathogens and maintaining immune system homeostasis are the two primary goals for sepsis therapy. To achieve these two goals simultaneously, rifampicin (Rif), a broad-spectrum antibiotic, was introduced into the LP-CaP NP (denoted as NAD⁺-Rif-LP-CaP) since NPs can improve antibiotics accumulation in the infected tissues via the enhanced permeability and retention (EPR) effect. Rif loaded in the lipid coating layer is released much faster than NAD⁺ encapsulated in the CaP core at pH 7.4 (FIG. 30). Thus, the NP can retain most of the NAD⁺ for intracellular delivery to mammalian cells while also effectively releasing the antibiotic in the infected tissue to kill the bacteria. Since bacteria can hardly take up NPs larger than 20 nm⁵⁶, our NP can specifically deliver NAD⁺ to mammalian cells rather than bacteria.

The therapeutic efficacy of NAD⁺-Rif-LP-CaP was evaluated using a polymicrobial blood infection model (FIG. 28D) because sepsis hosts are usually exposed to mixed bacterial infections. A mixture of methicillin-resistant S. aureus (MRSA) and P. aeruginosa, two multidrug resistant (MDR) pathogens identified as “serious threats” by Centers for Disease Control and Prevention (CDC) was administrated through tail vein (5×10⁷ CFU for each pathogen) to induce mouse blood infection and sepsis. One injection of different treatments (PBS, NAD⁺-LP-CaP, free Rif, Rif-LP-CaP, or NAD⁺-Rif-LP-CaP) were IV administrated 6 h after the infection. The treatments of NAD⁺-LP-CaP, free Rif, and Rif-LP-CaP all prolonged mice survival compared with the PBS group, but they did not significantly change the survival rate after two weeks (FIGS. 28E, 28F; FIG. 31). In contrast, NAD⁺-Rif-LP-CaP with both antimicrobial and immunomodulation capabilities resulted in a 90% mice survival after two weeks, demonstrating an excellent therapeutic outcome.

The bacterial colony forming units (CFUs) in mouse blood and different organs were measured 12 h after bacterial infection (FIG. 28G). While free Rif or Rif-loaded NPs reduced bacterial burden in most of the organs, NAD⁺-LP-CaP did not affect bacterial clearance. It is worth noting that mice treated with Rif-loaded NP (i.e., Rif-LP-CaP or NAD⁺-Rif-LP-CaP) had significantly lower CFU in liver and lung compared with those treated with free Rif. The finding can be attributed to the elevated NP accumulation in these organs, which was in line with the NP distribution data (FIGS. 15H, 15I; FIG. 17).

The therapeutic efficacy of NAD⁺-Rif-LP-CaP in preventing multiple organ failure, the hallmark of sepsis responsible for high mortality, was evaluated by blood biochemical analysis. The serum levels of alanine aminotransferase (ALT), aspartate aminotransferase (AST), alkaline phosphatase (ALP), and blood urea nitrogen (BUN) indicating liver and kidney damage were significantly increased after bacterial inoculation, suggesting severe organ injury (FIG. 28H; FIG. 32). NAD⁺-Rif-LP-CaP effectively protected the organs from infection induced damage and reduced the biochemical parameters to near baseline (FIG. 28H; FIG. 33). In contrast, free Rif and Rif-LP-CaP slightly mitigated the injury, while NAD⁺-LP-CaP, unable to inhibit bacterial growth, did not cause any improvement. Histopathological analysis was further used to substantiate the therapeutic benefit obtained from the biochemical data (FIG. 28I, FIG. 32). In the septic mice, hematoxylin and eosin (H&E)-stained sections revealed typical multiple organ injuries, e.g., congestion and dramatical inflammatory cells infiltration in the heart, spotty hepatocellular necrosis, nuclear debris in the spleen, alveolar wall thickening in the lung, severe congestion in the outer stripe of the outer medulla (OSOM) of the kidney, and tubular necrosis and cell sloughing in the inner stripe of the outer medulla (ISOM) of the kidney. Administration of NAD⁺-Rif-LP-CaP significantly attenuated the multiple organ injury, while NAD⁺-LP-CaP, free Rif, and Rif-LP-CaP did not show much efficacy. Finally, healthy mice subjected to three administrations of NAD⁺-LP-CaP, NAD⁺-Rif-LP-CaP, or NADH-LP-MOF every other day (containing 20 mg/kg NAD⁺ and 10 mg/kg Rif for each injection) via intravenous route did not show any significant organ damage monitored by tissue histological analysis, demonstrating the desirable biosafety of these NPs (FIG. 34).

### Example 8—Stability of NAD(H)-Loaded NPs

The stability of the NAD(H)-loaded NPs was studied (FIG. 42A-42B). Both types of the NPs were stable at 4° C. for one week. We also measured the sizes of the NPs lyophilized from an aqueous solution containing 10% sucrose as the cryoprotectant, and found the NPs maintained their stability for five weeks. Long-term storage stability is of great significance for clinical application and translation. Strategies such as introducing PEG onto the NP surface can further enhance the stability.

### ILLUSTRATIVE FEATURES

The present technology may include, but is not limited to, the features and combinations of features recited in the following numbered paragraphs, it being understood that the following paragraphs should not be interpreted as limiting the scope of the claims as appended hereto or mandating that all such features must necessarily be included in such claims:
P1. A nanoparticle comprising an inorganic core and NAD⁺ or NADH, coated with a lipid bilayer, wherein
   the inorganic core is selected from calcium phosphate or a metal organic framework (MOF);
   the MOF comprises a transition metal ion coordinated to a coordinating ligand, wherein the transition metal ion is selected from the group consisting of zinc, iron, zirconium, copper, and cobalt ions, and the coordinating ligand is selected from an imidazolate ligand or a carboxylate ligand; and
   the nanoparticle has an average hydrodynamic diameter of from at least 50 nm to less than 1000 nm.
P2. The nanoparticle of Paragraph P1, comprising 1 wt %-50 wt % NAD⁺ or NADH.
P3. The nanoparticle of Paragraph P1 or Paragraph P2, comprising 1 wt % to 25 wt % NAD⁺ or NADH.
P4. The nanoparticle of any one of Paragraphs P1-P3, comprising 10 wt %-50 wt % lipid bilayer.
P5. The nanoparticle of any one of Paragraphs P1-P4, wherein the lipid bilayer comprises lipids selected from the group consisting of of L-α-phosphatidylcholine (PC), 1,2-dioleoyl-sn-glycero-3-phosphate (DOPA), 1,2-dioleoyl-3-trimethylammonium-propane (DOTAP), 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phospho-L-serine (DOPS), 1,2-di stearoyl-sn-glycero-3-phosphoethanolamine-N-[methoxy(polyethylene glycol)] (D SPE-PEG), cholesterol, a cell membrane extracted from a red blood cell, macrophage, neutrophil or platelet, and combinations of two or more thereof.
P6. The nanoparticle of any one of Paragraphs P1-P5 wherein the lipid bilayer comprises lipids conjugated to poly(ethylene glycol) (PEG).
P7. The nanoparticle of any one of Paragraphs P1-P6, wherein up to 100 mol % of the lipids in the lipid bilayer are conjugated to PEG.
P8. The nanoparticle of any one of Paragraph P5-P7, wherein the lipids of the lipid bilayer comprise a combination of PC and DOPA, or PC and cholesterol.
P9. The nanoparticle of any one of Paragraphs P1-P8, wherein the lipid bilayer comprises DSPE-PEG wherein:
   the PEG has a free terminus selected from the group consisting of OH, O—C₁₋₄ alkyl ether, NH₂, NHR, COOH, COOR, wherein R is an alkyl or alkenyl group, a dye, a targeting ligand, and a metal chelating ligand, and
   the PEG has a number average molecular weight ranging from 300 to 10000 Da.
P10. The nanoparticle of any one of Paragraphs P1-P9, wherein the lipids of the lipid bilayer comprise a cell membrane extracted from a red blood cell, macrophage, neutrophil or platelet, and combinations of two or more thereof.
P11. The nanoparticle of any one of Paragraphs P1-P10, comprising 40-90 wt % inorganic core
P12. The nanoparticle any one of Paragraphs P1-P11, where the coordinating ligand is selected from the group consisting of imidazole, 2-methyl-imidazole, benzimidazole, 5-methylbenzimidazole, terephthalic acid, 2-methyl-pterphthalic acid, 2-hydroxy-terphthalic acid, and 2-amino-terphthalic acid, benzene-1,3,5-tricarboxylic acid, 1,3,5-tris(4-carboxyphenyl)benzene, 2,6-naphthalenedicarboxylic acid, 4,4′,4″-s-triazine-2,4,6-triyl-tribenzoic acid, and 2,5-dihydroxyterephthalic acid.
P13. The nanoparticle of any one of Paragraphs P1-P12, wherein the MOF comprises zinc ions and imidazolate ligands.
P14. The nanoparticle of Paragraph P13, wherein the imidazolate ligand is selected from imidazole, 2-methyl-imidazole, benzimidazole, or 5-methylbenzimidazole.
P15. The nanoparticle of Paragraph P13 or Paragraph P14 wherein the imidiazolate ligand is selected from 2-methyl-imidazole.
P16. The nanoparticle of any one of Paragraphs P1-P15Error! Reference source not found. further comprising an antimicrobial.
P17. The nanoparticle of Paragraph P16 wherein the antimicrobial is selected from the group consisting of rifampicin, cefepime, ciprofloxacin, minocycline, azithromycin, tigecycline, streptomycin, gentamicin, asmycin, etimicin, dacamycin, amikacin and combinations of any two or more thereof. In any embodiments, the antiviral agent may be selected from the group consisting of raltegravir, indinavir, nevirapine, sofosbuvir, amantadine, palivizumab, entecavir, lamivudine, ganciclovir, cidofovir, trifluridine, acyclovir, podofilox and combinations of any two or more thereof. In any embodiments, the antifungal agent may be selected from the group consisting of clotrimazole, econazole, micronazole, fluconazole, voriconazole, ketoconazole, terbinafine, amorolfine, isavuconazole, nystatin, echinocandin, nikkomycin Z, 5-flucytosine, tavaborole and combinations of any two or more thereof.
P18. The nanoparticle of any one of Paragraphs P1-P17, wherein the nanoparticle has an average hydrodynamic diameter of from 70 to 700 nm.
P19. A pharmaceutical composition comprising a nanoparticle of any one of Paragraphs
P1-P18 and a pharmaceutically acceptable carrier.
P20. A method of treating sepsis or inflammation comprising administering an effective amount of the nanoparticle of any one of Paragraphs P1-P18 to a subject suffering from sepsis or inflammation.
P21. The method of Paragraph P20, wherein the subject is a human.
P22. The method of Paragraph P20 or Paragraph P21, wherein the subject suffers from sepsis caused by a microbial infection and the method further comprises administering an effective amount of an antimicrobial to the subject.
P23. The method of Paragraph P22, wherein the sepsis is caused by a bacterial infection and the method further comprises administering an effective amount of an antibiotic to the subject separately, simultaneously, or sequentially with the nanoparticle.
P24. The method of Paragraph P22, wherein the sepsis is caused by a viral infection and the method further comprises administering an effective amount of an antiviral to the subject separately, simultaneously, or sequentially with the nanoparticle.
P25. The method of Paragraph P22, wherein the sepsis is caused by a fungal infection and the method further comprises administering an effective amount of an antifungal to the subject separately, simultaneously, or sequentially with the nanoparticle.
P26. The method of any one of Paragraphs P22 to P25, wherein the subject suffers from endotoxemia and/or drug-resistant or multi-drug resistant bacteremia.
P27. The method of any one of Paragraphs P22 to P25, wherein the subject suffers from septicemia.
P28. The method of any one of Paragraphs P20 to P26, wherein the subject suffers from a wound.
P29. The method of Paragraph P26, wherein the subject suffers from or is at risk of a secondary infection.
P30. The method of Paragraph P20 or Paragraph P21, wherein the amount is effective to increase cellular energy supply and/or inhibit cell apoptosis and dysfunction in immune cells, thereby reducing or preventing immunosuppression and/or endothelial damage.
P31. A method of decreasing a level of TNF-α or IL-6 in a cell or subject comprising administering an effective amount of the nanoparticle of any one of Paragraphs P1-P18 to the cell or subject.

### EQUIVALENTS

While certain embodiments have been illustrated and described, a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the phrase “consisting essentially of” will be understood to include those elements specifically recited and those additional elements that do not materially affect the basic and novel characteristics of the claimed technology. The phrase “consisting of” excludes any element not specified. The terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. More specifically, it will be understood that each use of terms such as “comprising,” “consisting essentially of,” or “consisting of”, discloses and provides written description and support for the use any of the other terms with the same or any other embodiment described herein.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A nanoparticle comprising an inorganic core and NAD⁺ or NADH, coated with a lipid bilayer, wherein
the inorganic core is selected from calcium phosphate or a metal organic framework (MOF);
the MOF comprises a transition metal ion coordinated to a coordinating ligand, wherein the transition metal ion is selected from the group consisting of zinc, iron, zirconium, copper, and cobalt ions, and the coordinating ligand is selected from an imidazolate ligand or a carboxylate ligand; and
the nanoparticle has an average hydrodynamic diameter of from at least 50 nm to less than 1000 nm.

2. The nanoparticle of claim 1, comprising 1 wt %-50 wt % NAD⁺ or NADH.

3. The nanoparticle of claim 1, comprising 1 wt % to 25 wt % NAD⁺ or NADH.

4. The nanoparticle of claim 1, comprising 10 wt %-50 wt % lipid bilayer.

5. The nanoparticle of claim 1, wherein the lipid bilayer comprises lipids selected from the group consisting of of L-α-phosphatidylcholine (PC), 1,2-dioleoyl-sn-glycero-3-phosphate (DOPA), 1,2-dioleoyl-3-trimethylammonium-propane (DOTAP), 1,2-dioleoyl-sn-glycero-3-phosphocholine (DOPC), 1,2-dioleoyl-sn-glycero-3-phosphoethanolamine (DOPE), 1,2-dioleoyl-sn-glycero-3-phospho-L-serine (DOPS), 1,2-distearoyl-sn-glycero-3-phosphoethanolamine-N-[methoxy(polyethylene glycol)] (DSPE-PEG), cholesterol, a cell membrane extracted from a red blood cell, macrophage, neutrophil or platelet, and combinations of two or more thereof.

6. The nanoparticle of claim 1, wherein the lipid bilayer comprises lipids conjugated to poly(ethylene glycol) (PEG).

7. The nanoparticle of claim 1, wherein up to 100 mol % of the lipids in the lipid bilayer are conjugated to PEG.

8. The nanoparticle of claim 5, wherein the lipids of the lipid bilayer comprise a combination of PC and DOPA, or PC and cholesterol.

9. The nanoparticle of claim 1, wherein the lipid bilayer comprises DSPE-PEG wherein:
the PEG has a free terminus selected from the group consisting of OH, O—C₁₋₄ alkyl ether, NH₂, NHR, COOH, COOR, wherein R is an alkyl or alkenyl group, a dye, a targeting ligand, and a metal chelating ligand, and
the PEG has a number average molecular weight ranging from 300 to 10000 Da.

10. The nanoparticle of claim 1, wherein the lipids of the lipid bilayer comprise a cell membrane extracted from a red blood cell, macrophage, neutrophil or platelet, and combinations of two or more thereof.

11. The nanoparticle of claim 1 comprising 40-90 wt % inorganic core.

12. The nanoparticle of claim 1, where the coordinating ligand is selected from the group consisting of imidazole, 2-methyl-imidazole, benzimidazole, 5-methylbenzimidazole, terephthalic acid, 2-methyl-pterphthalic acid, 2-hydroxy-terphthalic acid, and 2-amino-terphthalic acid, benzene-1,3,5-tricarboxylic acid, 1,3,5-tris(4-carboxyphenyl)benzene, 2,6-naphthalenedicarboxylic acid, 4,4′,4″-s-triazine-2,4,6-triyl-tribenzoic acid, and 2,5-dihydroxyterephthalic acid.

13. The nanoparticle of claim 1, wherein the MOF comprises zinc ions and imidazolate ligands.

14. The nanoparticle of claim 13, wherein the imidazolate ligand is selected from imidazole, 2-methyl-imidazole, benzimidazole, or 5-methylbenzimidazole.

15. The nanoparticle of claim 13, wherein the imidiazolate ligand is selected from 2-methyl-imidazole.

16. The nanoparticle of claim 1 further comprising an antimicrobial.

17. The nanoparticle of claim 16, wherein the antimicrobial is selected from the group consisting of rifampicin, cefepime, ciprofloxacin, minocycline, azithromycin, tigecycline, streptomycin, gentamicin, asmycin, etimicin, dacamycin, amikacin and combinations of any two or more thereof. In any embodiments, the antiviral agent may be selected from the group consisting of raltegravir, indinavir, nevirapine, sofosbuvir, amantadine, palivizumab, entecavir, lamivudine, ganciclovir, cidofovir, trifluridine, acyclovir, podofilox and combinations of any two or more thereof. In any embodiments, the antifungal agent may be selected from the group consisting of clotrimazole, econazole, micronazole, fluconazole, voriconazole, ketoconazole, terbinafine, amorolfine, isavuconazole, nystatin, echinocandin, nikkomycin Z, 5-flucytosine, tavaborole and combinations of any two or more thereof.

18. The nanoparticle of claim 1, wherein the nanoparticle has an average hydrodynamic diameter of from 70 to 700 nm.

19. A pharmaceutical composition comprising a nanoparticle of claim 1 and a pharmaceutically acceptable carrier.

20. A method of treating sepsis or inflammation comprising administering an effective amount of the nanoparticle of claim 1 to a subject suffering from sepsis or inflammation.

21. The method of claim 20, wherein the subject is a human.

22. The method of claim 21, wherein the subject suffers from sepsis caused by a microbial infection and the method further comprises administering an effective amount of an antimicrobial to the subject.

23. The method of claim 22, wherein the sepsis is caused by a bacterial infection and the method further comprises administering an effective amount of an antibiotic to the subject separately, simultaneously, or sequentially with the nanoparticle.

24. The method of claim 22, wherein the sepsis is caused by a viral infection and the method further comprises administering an effective amount of an antiviral to the subject separately, simultaneously, or sequentially with the nanoparticle.

25. The method of claim 22, wherein the sepsis is caused by a fungal infection and the method further comprises administering an effective amount of an antifungal to the subject separately, simultaneously, or sequentially with the nanoparticle.

26. The method of claim 22, wherein the subject suffers from one or more of endotoxemia, drug-resistant or multi-drug resistant bacteremia, septicemia, a wound, or suffers from or is at risk of a secondary infection.

27. The method of claim 20, wherein the amount is effective to increase cellular energy supply and/or inhibit cell apoptosis and dysfunction in immune cells, thereby reducing or preventing immunosuppression and/or endothelial damage.

28. A method of decreasing a level of TNF-α or IL-6 in a cell or subject comprising administering an effective amount of the nanoparticle of claim 1 to the cell or subject.


SEQUENCE LISTING (68 sequences)

SEQ ID NO: 1 - protein, 4 aa, Unknown
  Description of Unknown alpha-v-beta-3 integrin ligand sequence
GRGD

SEQ ID NO: 2 - protein, 12 aa, Artificial Sequence
  Description of Artificial Sequence Synthetic peptide
DITWDQLWDLMK

SEQ ID NO: 3 - protein, 16 aa, Artificial Sequence
  Description of Artificial Sequence Synthetic peptide
KYDGDITWDQLWDLMK

SEQ ID NO: 4 - protein, 12 aa, Human immunodeficiency virus 1
GRKKRRQRRRPQ

SEQ ID NO: 5 - protein, 18 aa, Artificial Sequence
  Description of Artificial Sequence Synthetic peptide
KLALKLALKALKAALKLA

SEQ ID NO: 6 - protein, 15 aa, Drosophila sp.
RQIKWFQNRRMKWKK

SEQ ID NO: 7 - protein, 16 aa, Artificial Sequence
  Description of Artificial Sequence Synthetic peptide
RQIRIWFQNRRMRWRR

SEQ ID NO: 8 - protein, 17 aa, Artificial Sequence
  Description of Artificial Sequence Synthetic peptide
CSIPPEVKFNKPFVYLI

SEQ ID NO: 9 - protein, 13 aa, Rana temporaria
FVQWFSKFLGRIL

SEQ ID NO: 10 - protein, 10 aa, Maurus palmatus
GDCLPHLKLC

SEQ ID NO: 11 - protein, 18 aa, Mus sp.
LLIILRRRIRKQAHAHSK

SEQ ID NO: 12 - protein, 24 aa, Homo sapiens
LGTYTQDFNKFHTFPQTAIGVGAP

SEQ ID NO: 13 - protein, 30 aa, Unknown
  Description of Unknown Neurturin sequence
GAAEAAARVYDLGLRRLRQRRRLRRERVRA

SEQ ID NO: 14 - protein, 18 aa, Drosophila sp.
RQIKIWFQNRRMKWKKGG

SEQ ID NO: 15 - protein, 30 aa, Artificial Sequence
  Description of Artificial Sequence Synthetic polypeptide
RRRQRRKKRGGDIMGEWGNEIFGAIAGFLG

SEQ ID NO: 16 - protein, 11 aa, Human immunodeficiency virus 1
YGRKKRRQRRR

SEQ ID NO: 17 - protein, 18 aa, Unknown
  Description of Unknown SynB1 sequence
RGGRLSYSRRRFSTSTGR

SEQ ID NO: 18 - protein, 10 aa, Unknown
  Description of Unknown SynB3 sequence
RRLSYSRRRF

SEQ ID NO: 19 - protein, 11 aa, Artificial Sequence
  Description of Artificial Sequence Synthetic peptide
PIRRRKKLRRL

SEQ ID NO: 20 - protein, 12 aa, Artificial Sequence
  Description of Artificial Sequence Synthetic peptide
RRQRRTSKLMKR

SEQ ID NO: 21 - protein, 15 aa, Flock House virus
RRRRNRTRRNRRRVR

SEQ ID NO: 22 - protein, 19 aa, Brome Mosaic virus
KMTRAQRRAAARRNRWTAR

SEQ ID NO: 23 - protein, 13 aa, Unknown
  Description of Unknown Human T-cell leukemia virus II sequence
TRRQRTRRARRNR

SEQ ID NO: 24 - protein, 13 aa, Human immunodeficiency virus 1
GRKKRRQRRRPPQ

SEQ ID NO: 25 - protein, 13 aa, Artificial Sequence
  Description of Artificial Sequence Synthetic peptide
GRRRRRRRRRPPQ

SEQ ID NO: 26 - protein, 27 aa, Artificial Sequence
  Description of Artificial Sequence Synthetic peptide
GWTLNSAGYLLGKINLKALAALAKKIL

SEQ ID NO: 27 - protein, 27 aa, Homo sapiens
MGLGLHLLVLAAALQGAWSQPKKKRKV

SEQ ID NO: 28 - protein, 27 aa, Unknown
  Description of Unknown FBP sequence
GALFLGWLGAAGSTMGAWSQPKKKRKV

SEQ ID NO: 29 - protein, 27 aa, Artificial Sequence
  Description of Artificial Sequence Synthetic peptide
GALFLGFLGAAGSTMGAWSQPKKKRKV

SEQ ID NO: 30 - protein, 27 aa, Artificial Sequence
  Description of Artificial Sequence Synthetic peptide
GALFLGFLGAAGSTMGAWSQPKSKRKV

SEQ ID NO: 31 - protein, 21 aa, Artificial Sequence
  Description of Artificial Sequence Synthetic peptide
KETWWETWWTEWSQPKKKRKV

SEQ ID NO: 32 - protein, 21 aa, Artificial Sequence
  Description of Artificial Sequence Synthetic peptide
KETWFETWFTEWSQPKKKRKV

SEQ ID NO: 33 - protein, 17 aa, Artificial Sequence
  Description of Artificial Sequence Synthetic peptide
  SITE (1)..(17): This sequence may encompass 4-17 residues
RRRRRRRRRRRRRRRRR

SEQ ID NO: 34 - protein, 17 aa, Artificial Sequence
  Description of Artificial Sequence Synthetic peptide
  SITE (1)..(17): This sequence may encompass 4-17 residues
KKKKKKKKKKKKKKKKK

SEQ ID NO: 35 - protein, 13 aa, Artificial Sequence
  Description of Artificial Sequence Synthetic peptide
  MOD_RES (2)..(2): Aminocaproic acid
  MOD_RES (4)..(4): Aminocaproic acid
  MOD_RES (6)..(6): Aminocaproic acid
  MOD_RES (8)..(8): Aminocaproic acid
  MOD_RES (10)..(10): Aminocaproic acid
  MOD_RES (12)..(12): Aminocaproic acid
RXRXRXRXRXRXR

SEQ ID NO: 36 - protein, 13 aa, Artificial Sequence
  Description of Artificial Sequence Synthetic peptide
  MOD_RES (2)..(2): Aminobutyric acid
  MOD_RES (4)..(4): Aminobutyric acid
  MOD_RES (6)..(6): Aminobutyric acid
  MOD_RES (8)..(8): Aminobutyric acid
  MOD_RES (10)..(10): Aminobutyric acid
  MOD_RES (12)..(12): Aminobutyric acid
RXRXRXRXRXRXR

SEQ ID NO: 37 - protein, 13 aa, Artificial Sequence
  Description of Artificial Sequence Synthetic peptide
RGRGRGRGRGRGR

SEQ ID NO: 38 - protein, 13 aa, Artificial Sequence
  Description of Artificial Sequence Synthetic peptide
RMRMRMRMRMRMR

SEQ ID NO: 39 - protein, 13 aa, Artificial Sequence
  Description of Artificial Sequence Synthetic peptide
RTRTRTRTRTRTR

SEQ ID NO: 40 - protein, 13 aa, Artificial Sequence
  Description of Artificial Sequence Synthetic peptide
RSRSRSRSRSRSR

SEQ ID NO: 41 - protein, 10 aa, Artificial Sequence
  Description of Artificial Sequence Synthetic peptide
RRRRRRRRRR

SEQ ID NO: 42 - protein, 13 aa, Artificial Sequence
  Description of Artificial Sequence Synthetic peptide
RARARARARARAR

SEQ ID NO: 43 - protein, 7 aa, Artificial Sequence
  Description of Artificial Sequence Synthetic peptide
RRRRRRR

SEQ ID NO: 44 - protein, 4 aa, Artificial Sequence
  Description of Artificial Sequence Synthetic probe
YVAD

SEQ ID NO: 45 - DNA, 22 bp, Artificial Sequence
  Description of Artificial Sequence Synthetic primer
gtaccgcaaacgcttctccctg

SEQ ID NO: 46 - DNA, 19 bp, Artificial Sequence
  Description of Artificial Sequence Synthetic primer
tggatgggtttgctgggat

SEQ ID NO: 47 - DNA, 23 bp, Artificial Sequence
  Description of Artificial Sequence Synthetic primer
aggctcctcacgcttgggtcttg

SEQ ID NO: 48 - DNA, 21 bp, Artificial Sequence
  Description of Artificial Sequence Synthetic primer
gccacttgccaggtctacgag

SEQ ID NO: 49 - DNA, 20 bp, Artificial Sequence
  Description of Artificial Sequence Synthetic primer
actgccgggatggctactat

SEQ ID NO: 50 - DNA, 20 bp, Artificial Sequence
  Description of Artificial Sequence Synthetic primer
ctggcagctcttctcaaagc

SEQ ID NO: 51 - DNA, 18 bp, Artificial Sequence
  Description of Artificial Sequence Synthetic primer
tgggcctcaaaggaaaga

SEQ ID NO: 52 - DNA, 19 bp, Artificial Sequence
  Description of Artificial Sequence Synthetic primer
acaaggcacgggacctatg

SEQ ID NO: 53 - DNA, 20 bp, Artificial Sequence
  Description of Artificial Sequence Synthetic primer
agacctgcttacttgctgtg

SEQ ID NO: 54 - DNA, 21 bp, Artificial Sequence
  Description of Artificial Sequence Synthetic primer
ccatcggcctttgagaaagtg

SEQ ID NO: 55 - DNA, 20 bp, Artificial Sequence
  Description of Artificial Sequence Synthetic primer
cggttccctaaacttcacga

SEQ ID NO: 56 - DNA, 20 bp, Artificial Sequence
  Description of Artificial Sequence Synthetic primer
ggtattgagacagacagtgg

SEQ ID NO: 57 - DNA, 23 bp, Artificial Sequence
  Description of Artificial Sequence Synthetic primer
cctccaaaggtgctcggcttcca

SEQ ID NO: 58 - DNA, 21 bp, Artificial Sequence
  Description of Artificial Sequence Synthetic primer
ctgcgtgtagcgactgttgag

SEQ ID NO: 59 - DNA, 21 bp, Artificial Sequence
  Description of Artificial Sequence Synthetic primer
ctgcggggagccattttggtg

SEQ ID NO: 60 - DNA, 22 bp, Artificial Sequence
  Description of Artificial Sequence Synthetic primer
aggcctgcacaatgatgacttt

SEQ ID NO: 61 - DNA, 21 bp, Artificial Sequence
  Description of Artificial Sequence Synthetic primer
tctggattcgctggctaatgg

SEQ ID NO: 62 - DNA, 21 bp, Artificial Sequence
  Description of Artificial Sequence Synthetic primer
tccaggtcatagagaggctca

SEQ ID NO: 63 - DNA, 18 bp, Artificial Sequence
  Description of Artificial Sequence Synthetic primer
ggtgctgatgtaccagtt

SEQ ID NO: 64 - DNA, 21 bp, Artificial Sequence
  Description of Artificial Sequence Synthetic primer
tcccagtcagtcctggaaatg

SEQ ID NO: 65 - DNA, 21 bp, Artificial Sequence
  Description of Artificial Sequence Synthetic primer
ggagggatggtgtatggtaag

SEQ ID NO: 66 - DNA, 22 bp, Artificial Sequence
  Description of Artificial Sequence Synthetic primer
acacatgaataacggggtttcc

SEQ ID NO: 67 - DNA, 20 bp, Artificial Sequence
  Description of Artificial Sequence Synthetic primer
gcaccatttcatttgtcacg

SEQ ID NO: 68 - DNA, 20 bp, Artificial Sequence
  Description of Artificial Sequence Synthetic primer
catgggatctgtttctttgc